(19) United States
(12) Patent Application Publication
(10) Pub. No.: US 2022/0346650 A1
(43) Pub. Date: Nov. 3, 2022

(54) SYSTEMS AND METHODS FOR VASCULAR AND STRUCTURAL IMAGING

(71) Applicant: Blaze Bioscience, Inc., Seattle, WA (US)

(72) Inventors: Julia Novak, Sequim, WA (US); Teri Dee Koller, Bainbridge Island, WA (US); Jeffrey Perry, Sunnyvale, CA (US); David Kittle, Cherryville (CA); Pramod Butte, Studio City, CA (US); Dennis Miller, Seattle, WA (US)

(21) Appl. No.: 17/764,131

(22) PCT Filed: Oct. 1, 2020
(86) PCT No.: PCT/US20/53746

§ 371 Date: Mar. 25, 2022

Related U.S. Application Data
(60) Provisional application No. 62/950,865, filed on Dec. 19, 2019.
(60) Provisional application No. 62/909,397, filed on Oct. 2, 2019.

(51) Int. Cl. A61B 5/00; A61B 5/02; A61B 5/026; G02B 27/10; G02B 27/30; G02B 21/06; G02B 21/36; G02B 21/16

(52) U.S. Cl. CPC A61B 5/0071; A61B 5/0035; A61B 5/0082; A61B 5/0036; A61B 5/02007; A61B 5/0261; G02B 27/1006; G02B 27/30; G02B 21/06; G02B 21/36; G02B 21/16; A61B 2562/223

(57) ABSTRACT

Disclosed herein are methods of vascular and structural imaging using imaging systems and methods described, the methods comprising producing an image of the vasculature or structure by imaging fluorescence using an imaging system, the system comprising: i) one or more detectors configured to form a fluorescence image of the sample and form a visible image of the sample; ii) a light source configured to emit an excitation light to induce fluorescence from the sample; and iii) a plurality of optics arranged to: direct the excitation light toward the sample; and direct a fluorescent light and a visible light from the sample to the detector; wherein the excitation light and the fluorescence light are directed substantially coaxially.

### CROSS-REFERENCE

The present application claims the benefit of U.S. Provisional Application No. 62/909,397, entitled “SYSTEMS AND METHODS FOR VASCULAR IMAGING,” filed on Oct. 2, 2019, and U.S. Provisional Application No. 62/950,865, entitled “SYSTEMS AND METHODS FOR VASCULAR AND STRUCTURAL IMAGING,” filed on Dec. 19, 2019, each of which applications are herein incorporated by reference in their entireties for all purposes.

### BACKGROUND

Fluorescence, including the use of fluorescent molecules tagged to other structures such as cells, nanoparticles, small molecules, antibodies, proteins, and peptides can be useful for organ, organ substructure, tissue and potentially cellular identification in medical imaging. Angiography or arteriography is a medical imaging technique used to visualize the inside, or lumen, of blood vessels and organs of the body, with particular interest in the arteries, veins, and the heart chamber. For example, fluorescent dyes can emit in visible (e.g., blue, green, yellow, red) and/or infrared, ultraviolet, or near infrared wavelengths. Although visible light fluorescence can be generally detected by naked eye, detection of ultraviolet light (UV), infrared (IR) light, and near infrared (NIR) light typically requires additional instrumentation for viewing. Infrared and near infrared can be a beneficial wavelength range for medical imaging. The benefits of infrared, near infrared and long wavelength visible light can be related to increased penetration depth, absence of significant intrinsic fluorescence, low absorption by blood (hemoglobin) or water. In medical applications it can be beneficial to have an imaging system which is capable of imaging both visible and infrared or near infrared images simultaneously, so that the surgeons can operate in tissues, for example, tagged with infrared fluorophore and do so seamlessly without having to switch between imaging modalities. The systems and methods of the present invention can be useful for many surgical applications, including vascular imaging, for example angiography, arteriography, perfusion imaging during reconstructive surgery, or vascular surgery; for visualization of abnormal tissues, of the vasculature or otherwise; for visualization of normal tissues to support surgical precision when preservation of the normal tissues is important to preserve function, for example ureters, parathyroid glands, or nerves; or to enhance the surgeon's ability to discriminate boundaries between segments of an organ, for example lobes of the liver.

Moreover, to image fluorescence from tissue, the imaging system will need to have ability and sensitivity to detect small amount of fluorescence, for example, from a fluorescent dye that flows through vasculature, is present in plasma or other physiologic structures, or adheres to or has been absorbed by the tissue. Traditionally, infrared fluorescence systems have used sensitive sensors to detect emitted infrared light, while using traditional halogen light sources for exciting the dye. Although such prior instrumentation can be able to produce images from such infrared light sources, sensitivity can be less than ideal due to inefficient halogen lighting as well as lower energy light sources surrounding excitation wavelengths, leading to inefficient and non-optimal infrared images. Although lasers have been used to achieve higher absorption and as a result increase fluorescence of the infrared or near infrared dyes, the images generated can be less than ideal in at least some instances.

Moreover, systems of present disclosure provide a method of extending the visibility window (i.e., the time in which fluorescence of the imaging or diagnostic agent is detectable by the system) in tissues and cells under physiologic or surgical conditions and in imaging, diagnostic and surgical applications. In addition, systems of present disclosure provide a method of reducing or using less imaging or diagnostic agent in imaging or diagnostic applications while still adequately detecting the fluorescence of the imaging or diagnostic agent. The increase in visibility window is proportional to the sensitivity of the system when optimized to the fluorophore measured, that is, the more sensitive the system the longer the fluorophore will be detected and hence the visibility window increased. This can also lead to improved contrast between diseased and normal tissue, for example when the imaging agent is cleared more quickly from the normal tissue than from the diseased tissue. In this instance the ability to lengthen the time between dose and imaging would enable better contrast during imaging. The methods herein provide increased sensitivity to fluorophore detection and a potential use related to general fluorophore imaging as well, for example, imaging an ICG or other imaging agent. The increased sensitivity may enable a longer time period of visibility which may reduce the need for multiple injections. The extended visibility window also would result in or require less overall drug exposure in vivo in a variety of imaging and diagnostic applications. Likewise, the reduction or use of less imaging or diagnostic agent is proportional to the sensitivity of the system when optimized to the fluorophore measured, that is, the more sensitive the system a lower the amount of fluorophore will be detected and hence the less imaging or diagnostic agent required for imaging, diagnostic and surgical applications. These advantages (i.e., increasing the visibility window and/or lowering the effective does of imaging or diagnostic agent) are desirable in imaging, diagnostic and surgical applications as they can enhance the time for detection of disease (by increasing visibility window) or reduce potential side effects of the agents (by reducing or lowering dose of such agents).

In addition, there is medical need of such systems in vascular imaging, angiography, arteriography, lymphography, cholangiography, and related methods used for identifying presence of health or disease, diagnostic imaging, health monitoring and the like. In neurosurgery, for example, such systems can be used to visualize blood flow and vessel patency during repair of vascular injury or malformation or during removal of tumors such as pituitary adenoma. In general surgery, such systems can be used for a variety of purposes, including visualization of blood flow and vessel patency, assessment of tissue perfusion, for example during reconstructive surgery, identification and management (sometimes preservation, sometimes selective resection) during surgery, visualization of organs or organ segments, identification of vital structures such as ureters and nerves, and detection of abnormal tissues such as endometriosis. Such systems are useful in imaging the vasculature of the eye as diagnostic and treatment support for various ophthalmic disorders, including glaucoma, Susac's syndrome, retinal vessel occlusion, macular edema or ischemia, macular degeneration, retinopathy, and the like. Such systems can be useful for endovascular imaging in atherosclerosis, to diagnose plaque instability and detect vessel restenosis. They can be useful in imaging tissue perfusion in chronic wounds, including diabetic wounds, and in critical limb ischemia. They can also be useful for treatment guidance, for example during injection of intra-arterial or intraocular chemotherapy.

### SUMMARY

In various aspects, the present disclosure provides a method of imaging a vasculature or structure in a sample from a subject, the method comprising producing an image of the vasculature or structure by imaging fluorescence using an imaging system, the system comprising: i) one or more detectors configured to form a fluorescence image of the sample and form a visible image of the sample; ii) a light source configured to emit an excitation light to induce fluorescence from the sample; and iii) a plurality of optics arranged to: direct the excitation light toward the sample; and direct a fluorescent light and a visible light from the sample to the detector; wherein the excitation light and the fluorescence light are directed substantially coaxially.

In some aspects, the fluorescence imaged is autofluorescence, a contrast or imaging agent, or any combination thereof. In some aspects, the method further comprises administering a contrast or imaging agent to the subject.

In various aspects, the present disclosure provides a method of imaging a vasculature or structure in a sample from a subject, the method comprising: a) administering a contrast or imaging agent to the subject; b) producing an image of the vasculature or structure by imaging the contrast or imaging agent using an imaging system, the system comprising: i) one or more detectors configured to form a fluorescence image of the sample and form a visible image of the sample; ii) a light source configured to emit an excitation light to induce fluorescence from the sample; and iii) a plurality of optics arranged to: direct the excitation light toward the sample; and direct a fluorescent light and a visible light from the sample to the detector; wherein the excitation light and the fluorescence light are directed substantially coaxially.

In some aspects, the excitation light comprises infrared light. In some aspects, the infrared light comprises near infrared light. In some aspects, the plurality of optics comprises a dichroic shortpass beam splitter to direct the infrared light and the visible light to the detector. In some aspects, the detector comprises a plurality of detectors and wherein the visible image comprises a color image. In some aspects, the plurality of detectors comprises a first detector to generate a color image and a second detector to generate the infrared image. In further aspects, the imaging system further comprises: a) a laser; b) an optical light guide coupled to a laser or narrow-band light source; c) a collimating lens into which the optical light guide ends; d) a laser clean-up filter; e) a dielectric mirror; f) a diffuser; g) a hole; or h) any combination thereof. In some aspects, the light source emits a wavelength absorbed by a fluorophore.

In some aspects, the light source is a narrow-band light source. In some aspects, the narrow-band light source generates light with a wavelength of from 700 nm to 800 nm, from 650 to 900 nm, from 700 nm to 900 nm, from 340 nm to 400 nm, from 360 to 420 nm, from 380 nm to 440 nm, or from 400 nm to 450 nm. In some aspects, the narrow-band light source emits light with a frequency visible by an NIR camera, and wherein the system further comprises a lens coupled to the optical light guide. In some aspects, the laser generates light with a wavelength of from 650 nm to 4000 nm, from 700 nm to 3000 nm, or from 340 nm to 450 nm. In some aspects, the laser generates light with a wavelength of from 750 nm to 950 nm, from 760 nm 825 nm, from 775 nm to 795 nm, from 780 nm to 795 nm, from 785 nm to 795 nm, from 780 nm to 790 nm, from 785 nm to 792 nm, or from 790 nm to 795.

In some aspects, the collimating lens is configured to collimate the excitation light, the fluorescent light, and the visible light. In some aspects, the optical light guide is a fiber optic cable, a solid light guide, a plastic light guide, a liquid light guide, a waveguide, or any combination thereof. In some aspects, the laser clean-up filter is configured to reduce bandwidth of the excitation light. In some aspects, the light source comprises: a) a broadband light source; b) an optical light guide coupled to the broadband light source; or c) a combination of a) and b). In some aspects, the broadband light source comprises one or more LEDs, a Xenon bulb, a halogen bulb, one or more or lasers, sunlight, fluorescent lighting, or a combination thereof. In some aspects, the broadband light source emits a visible wavelength, a wavelength absorbed by a fluorophore, or a combination thereof.

In some aspects, the broadband light source emits light with a frequency visible by an NIR camera and wherein the system further comprises a lens coupled to the optical light guide. In some aspects, the imaging system comprises a plurality of light sources and further comprises one or more of the following to combine the plurality of light sources into a single coaxial path: a) an optical attenuator comprising a dichroic filter, a dichroic mirror, a shutter, or any combination thereof, b) a filter at each light source c) a clean-up filter for a wavelength range of the excitation light; d) a short-pass filter for a wavelength range of the excitation light; e) an optical light guide; or f) an illumination optic. In further aspects, the imaging system further comprises: a) a shortpass (SP) mirror; b) a longpass (LP) mirror; c) a combination thereof. In some aspects, the dielectric mirror is configured to reflect the excitation light such that excitation light and the reflected excitation light have an intersection angle of about 60 degrees to about 120 degrees.

In some aspects, wherein the dielectric mirror is configured to reflect the excitation light such that excitation light and reflected excitation light have an intersection angle of about 90 degrees. In some aspects, the diffuser is configured to diffuse the excitation light. In some aspects, the hole is configured to let pass at least part of the excitation light. In some aspects, the hole is in a near-infrared mirror. In some aspects, the hole has a shape, and a size, and wherein at least one of the shape of the hole and the size of the hole are configured to allow an even distribution illumination of the sample within a field of view of a microscope. In some aspects, the excitation light comprises blue or ultraviolet light.

In some aspects, the blue or ultraviolet light comprises a light having a wavelength of from about 10 nm to about 460 nm, from about 10 nm to about 400 nm, or from about 400 nm to about 460 nm. In some aspects, the plurality of optics comprises a dichroic shortpass beam splitter, wherein the dichroic shortpass beam splitter is configured to let pass light with a wavelength of at most 700 nm with 90% to 95% efficiency at one or more specified angles of incidence. In some aspects, the one or more specific angles is within a range from 30 to 150 degrees. In some aspects, the visible light is directed from a microscope, an endoscope, an exoscope, a surgical robot, or an operating room lighting external to the imaging system.

In some aspects, the imaging system further comprises a locking key configured to securely lock an imaging head onto the microscope. In some aspects, the plurality of optics further comprises a secondary dichroic shortpass beam splitter. In some aspects, the system further comprises a dichroic longpass beam splitter. In some aspects, the excitation light and the fluorescence light substantially overlap at the beam splitter. In some aspects, substantially coaxially comprises an intersection angle of two optical paths to be less than 20 degrees, 15 degrees, 10 degrees, 5 degrees, 2 degrees, or 1 degree. In further aspects, the imaging system further comprises a physical attenuator configured to block an ambient light from one, two, or more of: the detector, the light source, and the plurality of optics.

In some aspects, the physical attenuator comprises a shield, a hood, a sleeve, a light shroud, or a baffle. In some aspects, the imaging system further comprises an Application Specific Integrated Circuit (ASIC) or a processor, wherein at least one of the ASIC and the processor is configured with instructions to generate a composite image of the sample, the composite image comprising the fluorescence image overlaid with the visible image. In some aspects, the contrast or imaging agent comprises a dye, a fluorophore, a fluorescent biotin compound, a luminescent compound, a chemiluminescent compound, or any combination thereof. In further aspects, the contrast or imaging agent absorbs a wavelength between from about 200 mm to about 900 mm.

In some aspects, the contrast or imaging agent comprises DyLight-680, DyLight-750, VivoTag-750, DyLight-800, IRDye-800, VivoTag-680, Cy5.5, or an indocyanine green (ICG) and any derivative of the foregoing; fluorescein and fluorescein dyes (e.g., fluorescein isothiocyanineo rF ITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, rythrosine, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin, coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), 7-aminocoumarin, a dialkylaminocoumarin reactive dye, 6,8-difluoro-7-hydroxycoumarin fluorophore, a hydroxycoumarin derivative, an alkoxycoumarin derivatives, a succinimidyl ester, a pyrene succinimidyl ester, a pyridyloxazole derivative, an aminonaphthalene-based dyes, dansyl chlorides, a dapoxyl dye, Dapoxyl sulfonyl chloride, amine-reactive Dapoxyl succinimidyl ester, carboxylic acid-reactive Dapoxyl (2-aminoethyl)sulfonamide), a bimane dye, bimane mercaptoacetic acid, an NBD dye, a QsY 35, or any combination thereof.

In some aspects, the administering comprises intravenous administration, intramuscular administration, subcutaneous administration, intraocular administration, intra-arterial administration, peritoneal administration, intratumoral administration, intradermal administration, or any combination thereof. In some aspects, the imaging comprises tissue imaging, ex vivo imaging, intraoperative imaging, or any combination thereof. In some aspects, the sample is in an in vivo sample, an ex vivo sample, or an intraoperative sample. In further aspects, the sample is an organ, an organ substructure, a tissue, or a cell. In some aspects, the sample autofluoresces. In some aspects, autofluorescence of the sample comprises an ocular fluorophore, tryptophan, or protein present in a tumor or malignancy. In some aspects, the method is used to visualize vessel flow or vessel patency.

In some aspects, the vasculature or structure comprises a blood vessel, lymph vasculature, neuronal vasculature, or CNS structure. In some aspects, the imaging is angiography, arteriography, lymphography, or cholangiography. In some aspects, the imaging comprises detecting a vascular abnormality, vascular malformation, vascular lesion, organ or organ substructure, cancer or diseased region, tissue, structure or cell. In some aspects, the vascular abnormality, vascular malformation, or vascular lesion is an aneurysm, an arteriovenous malformation, a cavernous malformation, a venous malformation, a lymphatic malformation, a capillary telangiectasia, a mixed vascular malformation, a spinal dural arteriovenous fistula, or a combination thereof. In some aspects, the organ or organ substructure is brain, heart, lung, kidney, liver, or pancreas. In further aspects, the method further comprises performing surgery on the subject. In some aspects, the surgery comprises angioplasty, cardiovascular surgery, aneurysm repair, valve replacement, aneurysm surgery, arteriovenous malformation or cavernous malformation, a venous malformation surgery, a lymphatic malformation surgery, a capillary telangiectasia surgery, a mixed vascular malformation surgery, or a spinal dural arteriovenous fistula surgery, repair or bypass, arterial bypass, organ transplant, plastic surgery, eye surgery, reproductive system surgery, stent insertion or replacement, plaque ablation, removing the cancer or the diseased region, tissue, structure or cell of the subject, or any combination thereof. In some aspects, the imaging comprises imaging a vascular abnormality, cancer or diseased region, tissue, structure, or cell of the subject after surgery. In further aspects, the method further comprises treating a cancer in the subject.

In some aspects, the method further comprises repair of an intracranial CNS vascular defect, a spinal CNS vascular defect; peripheral vascular defects; removal of abnormally vascularized tissue; ocular imaging and repair; anastomosis; reconstructive or plastic surgery; plaque ablation or treatment or restenosis in atherosclerosis; repair or resection (including selective resection), preservation (including selective preservation), of vital organs or structures such as nerves, kidney, thyroid, parathyroid, liver segments, or ureters; identification and management (sometimes preservation, sometimes selective resection) during surgery; diagnosis and treatment of ischemia in extremities; or treatment of chronic wounds. In some aspects, the intracranial vascular defect and/or the spinal vascular defect comprises an aneurysm, an arteriovenous malformation, a cavernous malformation, a venous malformation, a lymphatic malformation, a capillary telangiectasia, a mixed vascular malformation, or a spinal dural arteriovenous fistula, or any combination thereof. In some aspects, the peripheral vascular defect comprises an aneurysm, a coronary bypass, an other vascular bypass, a cavernous malformation, an arteriovenous malformation, a venous malformation, a lymphatic malformation, a capillary telangiectasia, a mixed vascular malformation, a spinal dural arteriovenous fistula, or any combination thereof. In some aspects, the abnormally vascularized tissue comprises endometriosis or a tumor.

In some aspects, the method further comprises radiology or fluorescence imaging using one or more of: an X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), microscope, operating microscope, confocal microscope, fluorescence scope, exoscope, surgical robot, surgical instrument, or any combination thereof. In some aspects, the method comprises measuring and/or quantitating fluorescence using one or more of a microscope, a confocal microscope, a fluorescence scope, an exoscope, a surgical robot, a surgical instrument, or any combination thereof. In some aspects, the imaging system is combined with or integrated into: a microscope, a confocal microscope, a fluorescence scope, an exoscope, a surgical robot, a surgical instrument, or any combination thereof. In some aspects, the imaging system comprises a microscope, a confocal microscope, a fluorescence scope, an exoscope, a surgical robot, a surgical instrument, or any combination thereof.

In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., RetinaVue 100, RetinaVue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPAL1 NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

In some aspects, the imaging system is configured to detect, image or assess a therapeutic agent; detect, image or assess a safety or a physiologic effect of the companion diagnostic agent; detect, image or assess a safety or a physiologic effect of the therapeutic agent; detect, image or assess a safety or a physiologic effect of the companion imaging agent; or any combination thereof. In some aspects, the contrast or imaging agent's safety or physiologic effect is bioavailability, uptake, concentration, presence, distribution and clearance, metabolism, pharmacokinetics, localization, blood concentration, tissue concentration, ratio, measurement of concentrations in blood or tissues, therapeutic window, extended visibility window, range and optimization, or any combination thereof. In some aspects, the method comprises administering a companion diagnostic agent, a therapeutic agent, or an imaging agent, and wherein the imaging comprises detecting the companion diagnostic agent, the therapeutic agent, or the imaging agent. In some aspects, the companion diagnostic agent, the therapeutic agent, or the imaging agent comprises a chemical agent, a radiolabel agent, radiosensitizing agent, fluorophore, therapeutic agent, an imaging agent, a diagnostic agent, a protein, a peptide, or a small molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. A better understanding of the features and advantages of the present subject matter will be obtained by reference to the following detailed description that sets forth illustrative embodiments and the accompanying drawings of which:

FIG. 1A shows an exemplary embodiment of the imaging systems and methods for simultaneous acquisition of infrared (IR) or near infrared (NIR) fluorescence and visible light herein with an operating microscope, in accordance with some embodiments;

FIG. 1B shows an exemplary composite image of fluorescent and visible imaging in tissue acquired using the imaging systems and methods, in accordance with some embodiments;

FIG. 2 shows an exemplary embodiment of a dichroic filter, in accordance with some embodiments;

FIG. 3A shows a schematic of an exemplary imaging system having non-coaxial illumination and imaging, in accordance with some embodiments;

FIG. 3B shows a schematic of an exemplary imaging system having coaxial illumination and imaging, in accordance with some embodiments;

FIG. 4 shows an exemplary embodiment of the imaging systems and methods capable of simultaneously acquiring both infrared or near infrared (NIR) fluorescence and visible light images; in this case, a two-camera system that can be attached to an operating microscope, in accordance with some embodiments;

FIG. 5A shows an illustration of a first exemplary single camera imaging system capable of simultaneously acquiring both infrared or near infrared (NIR) fluorescence and visible light images, in accordance with some embodiments;

FIG. 5B shows an illustration of a second exemplary single camera imaging system capable of simultaneously acquiring both infrared or near infrared (NIR) fluorescence and visible light images, in accordance with some embodiments;

FIG. 5C shows an illustration of a third exemplary single camera imaging system capable of simultaneously acquiring both infrared or near infrared (NIR) fluorescence and visible light images, in accordance with some embodiments;

FIG. 6A shows an illustration of a fourth exemplary single camera imaging system capable of simultaneously acquiring both infrared or near infrared (NIR) fluorescence and visible light images, in accordance with some embodiments;

FIG. 6B shows an illustration of a fifth exemplary single camera imaging system capable of simultaneously acquiring both infrared or near infrared (NIR) fluorescence and visible light images, in accordance with some embodiments;

FIG. 7A shows an illustration of a third exemplary single camera imaging system capable of simultaneously acquiring both infrared or near infrared (NIR) fluorescence and visible light images, in accordance with some embodiments;

FIG. 7B shows exemplary images captured using the imaging systems and methods herein;

FIG. 7C shows an exemplary image of shadow corrections due to thickness of dichroic filter(s), in accordance with some embodiments;

FIG. 7D shows a high magnification image of FIG. 7C;

FIG. 8A shows an exemplary imaging system and the path of the excitation light, in accordance with some embodiments;

FIG. 8B shows a high magnification image of FIG. 8A;

FIG. 9 shows an exemplary timing diagram the frame capture and laser on/off triggering for collection of infrared fluorescence images, near infrared (NIR) fluorescence images, and ambient light (dark background) images;

FIG. 10A shows an exemplary image of the fluorescent and visible light imaging in ex vivo tissue, wherein the near infrared (NIR) image has a pseudo color, and wherein the visible light is changed to black, in accordance with some embodiments.

FIG. 10B shows an exemplary image of the fluorescent and visible light imaging in ex vivo tissue, wherein the near infrared (NIR) image has a pseudo color, and wherein the visible light is changed to white, in accordance with some embodiments.

FIG. 10C shows an exemplary image of the fluorescent and visible light imaging in ex vivo tissue, wherein the near infrared (NIR) image has a pseudo color, and wherein the visible light is changed to red, in accordance with some embodiments.

FIG. 11 shows an exemplary image of a lock and a key for an imaging head, in accordance with some embodiments;

FIG. 12 shows an exemplary illustration of a two-camera imaging system which can be attached to an operating microscope for simultaneous acquisition of near infrared (NIR) fluorescence and visible light; in this case, a, in accordance with some embodiments;

FIG. 13 shows an exemplary schematic diagram of the method steps of using the image systems, in accordance with some embodiments;

FIG. 14 shows a non-limiting schematic diagram of a digital processing device; in this case, a device with one or more CPUs, a memory, a communication interface, and a display, in accordance with some embodiments;

FIG. 15A shows a first exemplary visible image of a tissue sample acquired using the imaging systems and methods herein, in accordance with some embodiments;

FIG. 15B shows a first exemplary NIR fluorescent image of a tissue sample acquired using the imaging systems and methods herein, in accordance with some embodiments;

FIG. 15C shows a first exemplary composite visible and fluorescent image of a tissue sample acquired using the imaging systems and methods herein, in accordance with some embodiments;

FIG. 15D shows a second exemplary visible image of a tissue sample acquired using the imaging systems and methods herein, in accordance with some embodiments;

FIG. 15E shows a second exemplary NIR fluorescent image of a tissue sample acquired using the imaging systems and methods herein, in accordance with some embodiments;

FIG. 15F shows a second exemplary composite visible and fluorescent image of a tissue sample acquired using the imaging systems and methods herein, in accordance with some embodiments;

FIG. 16 shows an illustration of an exemplary double camera imaging system capable of simultaneously acquiring both infrared or near infrared (NIR) fluorescence and visible light images, in accordance with some embodiments; and

FIG. 17 shows a non-limiting example of a computing device; in this case, a device with one or more processors, memory, storage, and a network interface.

FIG. 18 and FIG. 19 show exemplary visible images of a tissue sample of a vascular lesion, vascular malformation or vascular abnormality acquired using the imaging systems and methods herein, in accordance with some embodiments. Representative images of in situ or intra-operative tissue during surgery on a vascular lesion in a patient, wherein 22 mg (15 mg/m²) of tozuleristide was administered to the human subject.

FIG. 18A shows a near-infrared (NIR) image of the in situ specimen. Fluorescence signal, corresponding to lighter and brighter areas in the NIR images, is indicative of the presence of tozuleristide in the vascular lesion. Labeled arrows indicate non-fluorescent regions of normal blood vessels (“BV”) and normal brain tissue (“NB”). In contrast, fluorescence signal corresponding to lighter and brighter areas in the NIR image, was indicative of the presence of tozuleristide on the abnormal vascular lesion (“VL”), and not in normal tissue.

FIG. 18B shows the white light image corresponding to FIG. 18A that represents what the surgeon would normally see without fluorescence guidance. The arrows mark the same locations as shown in the NIR image in FIG. 18A. The vascular lesion (“VL”) had a similar appearance to the normal blood vessels (“BV”) in this image.

FIG. 18C shows the NIR fluorescence and white light composite image of FIG. 18A and FIG. 18B, with arrows marking the same locations as shown in FIG. 18A and FIG. 18B. Fluorescence in the vascular lesion (“VL”) clearly differentiated it from the surrounding normal tissues, including normal blood vessels (“BV”).

FIG. 19A shows a near-infrared (NIR) image of the vascular lesion during the surgery. Arrows indicate the vascular lesion (labeled “VL”) and adjacent normal brain (labeled “NB”), which is non-fluorescent.

FIG. 19B shows the white light image corresponding to FIG. 19A. While the normal brain has a light tan to pink color (light gray in a gray scale image), it is perfused with normal blood vessels that can be differentiated from the vascular lesion by the absence of fluorescence.

FIG. 19C shows the composite white light and NIR image shown in FIG. 19A and FIG. 19B.

### DETAILED DESCRIPTION

Systems and methods used herein can be used in vascular imaging, angiography, arteriography, lymphography, cholangiography, and related methods for identifying the presence of health or disease, diagnostic imaging, health monitoring and the like. In neurosurgery, for example, such systems can be used to visualize blood flow and vessel patency during repair of vascular injury or malformation or during removal of tumors such as pituitary adenoma. In general surgery, such systems can be used for a variety of purposes, including visualization of blood flow and vessel patency, assessment of tissue perfusion, visualization of organs or organ segments, identification of vital structures such as ureters and nerves, and detection of abnormal tissues such as endometriosis. Such systems are useful in imaging the vasculature of the eye as diagnostic and treatment support for various ophthalmic disorders, including glaucoma, Susac's syndrome, retinal vessel occlusion, macular edema or ischemia, macular degeneration, retinopathy, and the like. Such systems can be useful for endovascular imaging in atherosclerosis, to diagnose plaque instability and detect vessel restenosis. They can be useful in imaging tissue perfusion in chronic wounds, including diabetic wounds, and in critical limb ischemia. They can also be useful for treatment guidance, for example during injection of intra-arterial or intraocular chemotherapy.

Some prior systems for generating fluorescence images require a greater control over visible lighting than is generally available to allow measurement of fluorescence signals. However, in some cases, complete or partial control over the visible lighting is not readily available or ideal, for example in a surgical suite or other area where surgeons will adjust light for their needs to view tissue, which can be less than ideal for measuring fluorescence signals. Additionally, in situations where the surgery is conducted using a surgical microscope, it can be possible to control the illumination by repositioning the microscope in order to image the fluorescence signal from surgical tissues, and then replacing it to its original position to resume operating when the fluorescence imaging is complete. Moreover, with sources such as halogen lamps the absorption of excitation light by the fluorophore is sub-optimum and thus such systems cannot be able achieve simultaneous recording in real time or at video rate without any perceivable lag (e.g., no more than about 100 ms). Further, the prior systems for visible and infrared or near infrared imaging can disrupt the surgical techniques. For example, the surgeon may not be able to use the microscope in the traditional way (e.g., viewing through the eye pieces) when the fluorescence is measured. One problem which can arise with prior systems is that the viewing angles of the fluorescence stimulation or emission wavelengths and the visible wavelengths of the operating microscope can be less than ideally arranged, which can result in less than ideal optical signals and image registration resulting in sub-optimal, unclear or poor images. Also, the fluorescence signal can exhibit “blind spots” in some prior systems, such that the tissue does not visibly fluoresce and appears normal and non-cancerous, resulting in failure to identify critical cancerous tissue during surgery in at least some instances.

In light of the above, there is a need for systems and methods that overcome at least some of the aforementioned disadvantages of the prior systems. Ideally such systems and methods would provide fluorescence and visible imaging together, for example simultaneously, with an operating microscope. Moreover, there is a need for systems that do not rely on repositioning the operating microscope to view fluorescence and visible images, and provide imaging of the surgical area together with the fluorescence imaging system during operations and/or pathological examination.

The systems and methods disclosed herein are well suited for combination with many types of surgical and other procedures with minimal disruption in workflow. For example, the presently disclosed methods and apparatus are well suited for incorporation with prior operating microscopes, and other imaging devices, such as cameras, monitors, exoscopes, surgical robots, endoscopes, in order to improve the surgical work flow. In some embodiments, the systems and methods disclosed herein are capable of simultaneous capture of visible light and infrared fluorescence and can either be used stand-alone (e.g. open field or endoscopic) or as an attachment to a surgical instrument, such as an operating microscope. For example, the methods and apparatus disclosed herein are well suited for combination and incorporation with commercially available operating microscopes known to one of ordinary skill in the art, such as those commercially available from such companies and sources as Zeiss, Leica, Intuitive Surgical, and Haag-Streight, and each of their affiliates. The methods and apparatus can be combined with commercially available surgical robotic systems and endoscopes known to one of ordinary skill in the art, such as, for example, those commercially available from Intuitive Surgical, and its affiliates.

### Imaging Systems

Provided herein are imaging systems and methods for detecting fluorophore emissions. The imaging system can comprise: a detector, a light source, and a plurality of optics. The detector can be configured to form a fluorescence image of the sample, to form a visible image of the sample, or both. The light source can be configured to emit an excitation light. The excitation light can induce fluorescence of the sample. The plurality of optics can be arranged to direct the excitation light toward the sample, direct a fluorescent light and a visible light from the sample to the detector, or both. The excitation light and the fluorescence light can be directed substantially coaxially.

Fluorophores can be conjugated or fused to another moiety as described herein and be used to home, target, migrate to, be retained by, accumulate in, and/or bind to, or be directed to specific organs, substructures within organs, tissues, targets or cells and used in conjunction with the systems and methods herein. The fluorophore emission can comprise an infrared, near infrared, blue or ultraviolet emission.

In some embodiments, the system is configured to detect fluorophores have an absorption wavelength of about 10 nm to about 200 nm. In some embodiments, the system is configured to detect fluorophores have an absorption wavelength of about 10 nm to about 20 nm, about 10 nm to about 30 nm, about 10 nm to about 40 nm, about 10 nm to about 50 nm, about 10 nm to about 75 nm, about 10 nm to about 100 nm, about 10 nm to about 125 nm, about 10 nm to about 150 nm, about 10 nm to about 200 nm, about 20 nm to about 30 nm, about 20 nm to about 40 nm, about 20 nm to about 50 nm, about 20 nm to about 75 nm, about 20 nm to about 100 nm, about 20 nm to about 125 nm, about 20 nm to about 150 nm, about 20 nm to about 200 nm, about 30 nm to about 40 nm, about 30 nm to about 50 nm, about 30 nm to about 75 nm, about 30 nm to about 100 nm, about 30 nm to about 125 nm, about 30 nm to about 150 nm, about 30 nm to about 200 nm, about 40 nm to about 50 nm, about 40 nm to about 75 nm, about 40 nm to about 100 nm, about 40 nm to about 125 nm, about 40 nm to about 150 nm, about 40 nm to about 200 nm, about 50 nm to about 75 nm, about 50 nm to about 100 nm, about 50 nm to about 125 nm, about 50 nm to about 150 nm, about 50 nm to about 200 nm, about 75 nm to about 100 nm, about 75 nm to about 125 nm, about 75 nm to about 150 nm, about 75 nm to about 200 nm, about 100 nm to about 125 nm, about 100 nm to about 150 nm, about 100 nm to about 200 nm, about 125 nm to about 150 nm, about 125 nm to about 200 nm, or about 150 nm to about 200 nm. In some embodiments, the system is configured to detect fluorophores have an absorption wavelength of about 10 nm, about 20 nm, about 30 nm, about 40 nm, about 50 nm, about 75 nm, about 100 nm, about 125 nm, about 150 nm, or about 200 nm. In some embodiments, the system is configured to detect fluorophores have an absorption wavelength of at least about 10 nm, about 20 nm, about 30 nm, about 40 nm, about 50 nm, about 75 nm, about 100 nm, about 125 nm, or about 150 nm. In some embodiments, the system is configured to detect fluorophores have an absorption wavelength of at most about 20 nm, about 30 nm, about 40 nm, about 50 nm, about 75 nm, about 100 nm, about 125 nm, about 150 nm, or about 200 nm.

In some embodiments, the systems and methods herein detect fluorophore emissions. The fluorophores emissions can comprise an ultraviolet emission. The ultraviolet emissions can have a wavelength from 10 nm to 400 nm, and up to 450 nm or 460 nm into the blue light spectrum, including fluorophores with absorption wavelengths in the ranges disclosed herein, including 10-20 nm, 20-30 nm, 30-40 nm, 40-50 nm, 50-60 nm, 60-70 nm, 70-80 nm, 80-90 nm, 90-100 nm, 100-110 nm, 110-120 nm, 120-130 nm, 130-140 nm, 140-150 nm, 150-160 nm, 160-170 nm, 170-180 nm, 180-190 nm, 190-200 nm, 200-210 nm, 210-220 nm, 220-230 nm, 230-240 nm, 240-250 nm, 250-260 nm, 260-270 nm, 270-280 nm, 280-290 nm, 290-300 nm, 300-310 nm, 310-320 nm, 320-330 nm, 330-340 nm, 340-350 nm, 350-360 nm, 360-370 nm, 370-380 nm, 380-390 nm, 390-400 nm, 400-410 nm, 410-420 nm, 420-430 nm, 430-440 nm, 440-450 nm, 450-460 nm, 300-350 nm, 325-375 nm, 350-400 nm, 400-450 nm, a wavelength in the range of 340 nm to 400 nm, 360 to 420 nm, 380 nm to 440 nm, 400 nm to 450 nm, 400 nm to 460 nm or any wavelength within any of these foregoing ranges.

In some embodiments, the system is configured to detect fluorophores have an absorption wavelength of about 200 nm to about 1,000 nm. In some embodiments, the system is configured to detect fluorophores have an absorption wavelength of about 200 nm to about 250 nm, about 200 nm to about 300 nm, about 200 nm to about 350 nm, about 200 nm to about 400 nm, about 200 nm to about 450 nm, about 200 nm to about 500 nm, about 200 nm to about 600 nm, about 200 nm to about 700 nm, about 200 nm to about 800 nm, about 200 nm to about 900 nm, about 200 nm to about 1,000 nm, about 250 nm to about 300 nm, about 250 nm to about 350 nm, about 250 nm to about 400 nm, about 250 nm to about 450 nm, about 250 nm to about 500 nm, about 250 nm to about 600 nm, about 250 nm to about 700 nm, about 250 nm to about 800 nm, about 250 nm to about 900 nm, about 250 nm to about 1,000 nm, about 300 nm to about 350 nm, about 300 nm to about 400 nm, about 300 nm to about 450 nm, about 300 nm to about 500 nm, about 300 nm to about 600 nm, about 300 nm to about 700 nm, about 300 nm to about 800 nm, about 300 nm to about 900 nm, about 300 nm to about 1,000 nm, about 350 nm to about 400 nm, about 350 nm to about 450 nm, about 350 nm to about 500 nm, about 350 nm to about 600 nm, about 350 nm to about 700 nm, about 350 nm to about 800 nm, about 350 nm to about 900 nm, about 350 nm to about 1,000 nm, about 400 nm to about 450 nm, about 400 nm to about 500 nm, about 400 nm to about 600 nm, about 400 nm to about 700 nm, about 400 nm to about 800 nm, about 400 nm to about 900 nm, about 400 nm to about 1,000 nm, about 450 nm to about 500 nm, about 450 nm to about 600 nm, about 450 nm to about 700 nm, about 450 nm to about 800 nm, about 450 nm to about 900 nm, about 450 nm to about 1,000 nm, about 500 nm to about 600 nm, about 500 nm to about 700 nm, about 500 nm to about 800 nm, about 500 nm to about 900 nm, about 500 nm to about 1,000 nm, about 600 nm to about 700 nm, about 600 nm to about 800 nm, about 600 nm to about 900 nm, about 600 nm to about 1,000 nm, about 700 nm to about 800 nm, about 700 nm to about 900 nm, about 700 nm to about 1,000 nm, about 800 nm to about 900 nm, about 800 nm to about 1,000 nm, or about 900 nm to about 1,000 nm. In some embodiments, the system is configured to detect fluorophores have an absorption wavelength of about 200 nm, about 250 nm, about 300 nm, about 350 nm, about 400 nm, about 450 nm, about 500 nm, about 600 nm, about 700 nm, about 800 nm, about 900 nm, or about 1,000 nm. In some embodiments, the system is configured to detect fluorophores have an absorption wavelength of at least about 200 nm, about 250 nm, about 300 nm, about 350 nm, about 400 nm, about 450 nm, about 500 nm, about 600 nm, about 700 nm, about 800 nm, or about 900 nm. In some embodiments, the system is configured to detect fluorophores have an absorption wavelength of at most about 250 nm, about 300 nm, about 350 nm, about 400 nm, about 450 nm, about 500 nm, about 600 nm, about 700 nm, about 800 nm, about 900 nm, or about 1,000 nm.

In some embodiments, the system is configured to detect fluorophores have an absorption wavelength of about 1,000 nm to about 4,000 nm. In some embodiments, the system is configured to detect fluorophores have an absorption wavelength of about 1,000 nm to about 1,250 nm, about 1,000 nm to about 1,500 nm, about 1,000 nm to about 1,750 nm, about 1,000 nm to about 2,000 nm, about 1,000 nm to about 2,250 nm, about 1,000 nm to about 2,500 nm, about 1,000 nm to about 2,750 nm, about 1,000 nm to about 3,000 nm, about 1,000 nm to about 3,250 nm, about 1,000 nm to about 3,500 nm, about 1,000 nm to about 4,000 nm, about 1,250 nm to about 1,500 nm, about 1,250 nm to about 1,750 nm, about 1,250 nm to about 2,000 nm, about 1,250 nm to about 2,250 nm, about 1,250 nm to about 2,500 nm, about 1,250 nm to about 2,750 nm, about 1,250 nm to about 3,000 nm, about 1,250 nm to about 3,250 nm, about 1,250 nm to about 3,500 nm, about 1,250 nm to about 4,000 nm, about 1,500 nm to about 1,750 nm, about 1,500 nm to about 2,000 nm, about 1,500 nm to about 2,250 nm, about 1,500 nm to about 2,500 nm, about 1,500 nm to about 2,750 nm, about 1,500 nm to about 3,000 nm, about 1,500 nm to about 3,250 nm, about 1,500 nm to about 3,500 nm, about 1,500 nm to about 4,000 nm, about 1,750 nm to about 2,000 nm, about 1,750 nm to about 2,250 nm, about 1,750 nm to about 2,500 nm, about 1,750 nm to about 2,750 nm, about 1,750 nm to about 3,000 nm, about 1,750 nm to about 3,250 nm, about 1,750 nm to about 3,500 nm, about 1,750 nm to about 4,000 nm, about 2,000 nm to about 2,250 nm, about 2,000 nm to about 2,500 nm, about 2,000 nm to about 2,750 nm, about 2,000 nm to about 3,000 nm, about 2,000 nm to about 3,250 nm, about 2,000 nm to about 3,500 nm, about 2,000 nm to about 4,000 nm, about 2,250 nm to about 2,500 nm, about 2,250 nm to about 2,750 nm, about 2,250 nm to about 3,000 nm, about 2,250 nm to about 3,250 nm, about 2,250 nm to about 3,500 nm, about 2,250 nm to about 4,000 nm, about 2,500 nm to about 2,750 nm, about 2,500 nm to about 3,000 nm, about 2,500 nm to about 3,250 nm, about 2,500 nm to about 3,500 nm, about 2,500 nm to about 4,000 nm, about 2,750 nm to about 3,000 nm, about 2,750 nm to about 3,250 nm, about 2,750 nm to about 3,500 nm, about 2,750 nm to about 4,000 nm, about 3,000 nm to about 3,250 nm, about 3,000 nm to about 3,500 nm, about 3,000 nm to about 4,000 nm, about 3,250 nm to about 3,500 nm, about 3,250 nm to about 4,000 nm, or about 3,500 nm to about 4,000 nm. In some embodiments, the system is configured to detect fluorophores have an absorption wavelength of about 1,000 nm, about 1,250 nm, about 1,500 nm, about 1,750 nm, about 2,000 nm, about 2,250 nm, about 2,500 nm, about 2,750 nm, about 3,000 nm, about 3,250 nm, about 3,500 nm, or about 4,000 nm. In some embodiments, the system is configured to detect fluorophores have an absorption wavelength of at least about 1,000 nm, about 1,250 nm, about 1,500 nm, about 1,750 nm, about 2,000 nm, about 2,250 nm, about 2,500 nm, about 2,750 nm, about 3,000 nm, about 3,250 nm, or about 3,500 nm. In some embodiments, the system is configured to detect fluorophores have an absorption wavelength of at most about 1,250 nm, about 1,500 nm, about 1,750 nm, about 2,000 nm, about 2,250 nm, about 2,500 nm, about 2,750 nm, about 3,000 nm, about 3,250 nm, about 3,500 nm, or about 4,000 nm.

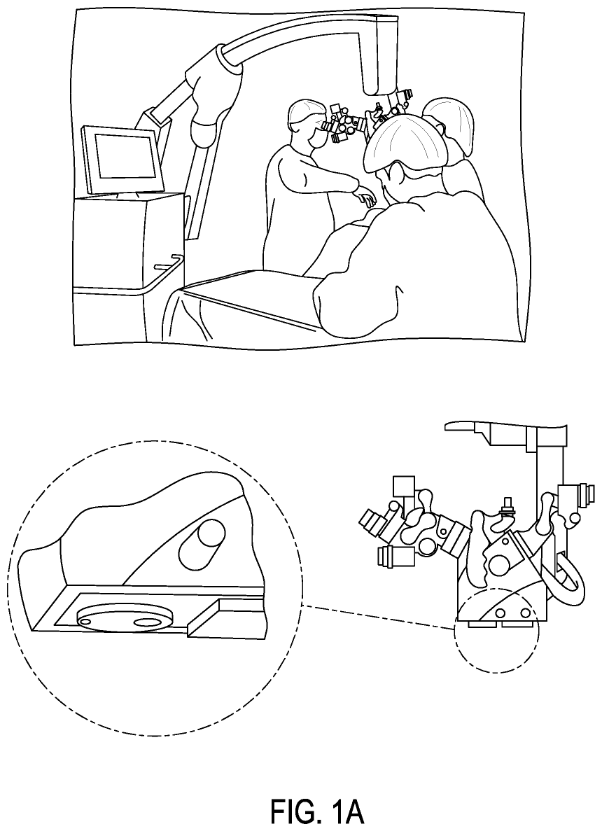

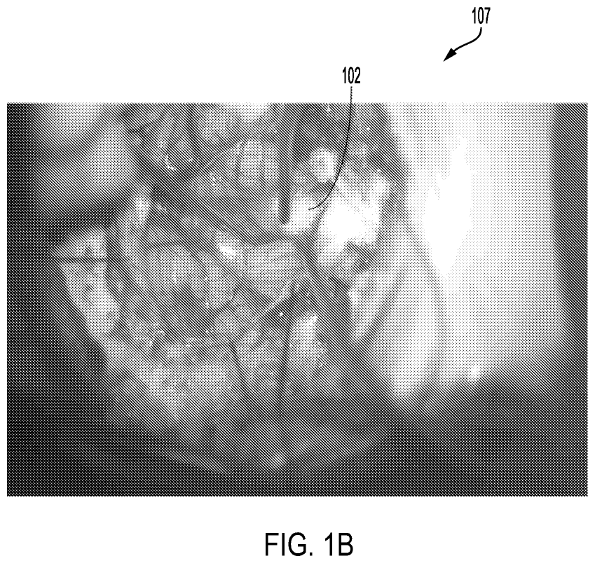

Referring to FIG. 1A, in a particular embodiment, the imaging system 100 herein is used with a microscope 101, e.g. a surgical microscope, for simultaneous imaging of fluorescence signal and visible light from the tissue 105. In this embodiment, the illumination axis 103 of the fluorescence emission from the tissue is co-axial with the imaging axis 104. In other words, the excitation source's light is coaxial with an imaging axis of the imaging system 100 and/or the operating microscope 101. In this embodiment, the microscope includes a visible light source 101a for providing visible light to the imaging system.

FIG. 1B shows an exemplary image generated using the imaging systems and methods herein. In this particular embodiment, the fluorescent tissue 102 is near the center of the field of view of the image display 107. In this embodiment, the fluorescent image is superimposed on visible image and the superimposed composite image is displayed on an external monitor. A digital processing device or a processor is used for processing and combining the images for display. In some embodiments, the surgeon can directly view such visible and fluorescence images using the microscope. In some embodiments, the surgeon can view such images from a heads-up display in the operation room or any other device capable of displaying images.

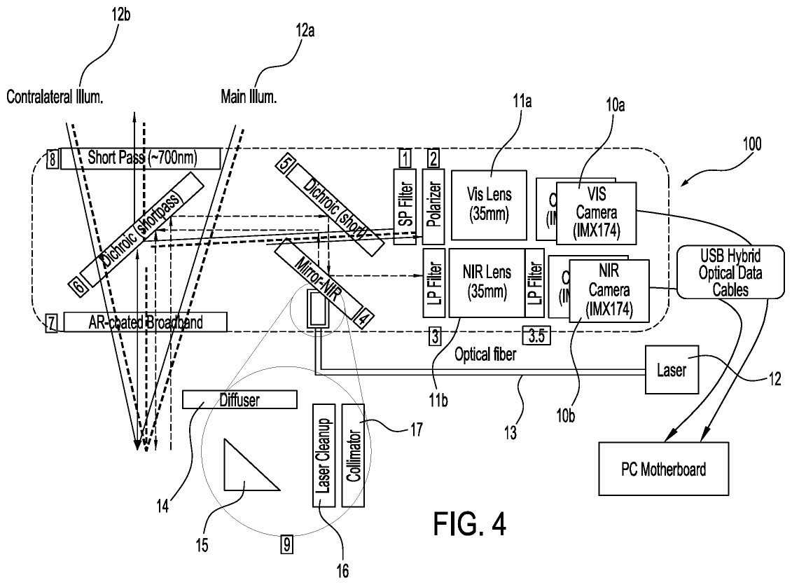

The imaging system can comprise a light source and one or more optical light guides. The light source and one or more optical light guides can be arranged to reduce the diffraction from the edges, and to reduce flooding of the NIR sensor with the excitation light, the illumination light, or both. Exemplary arrangements of the light source and the optical light guise are shown in FIGS. 4, 5A-5C, 6A-6B, 7A, and FIG. 16.

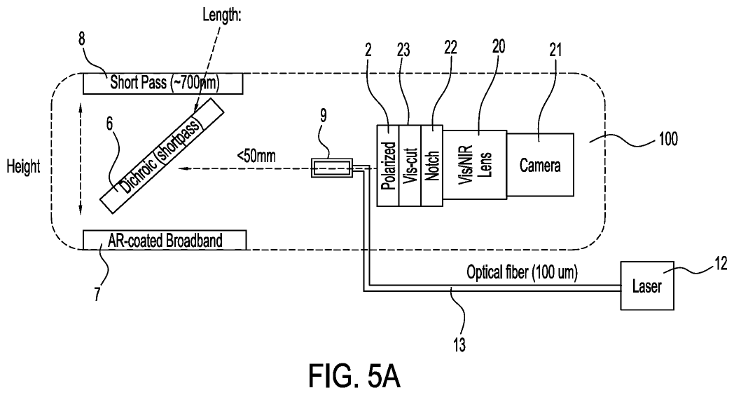

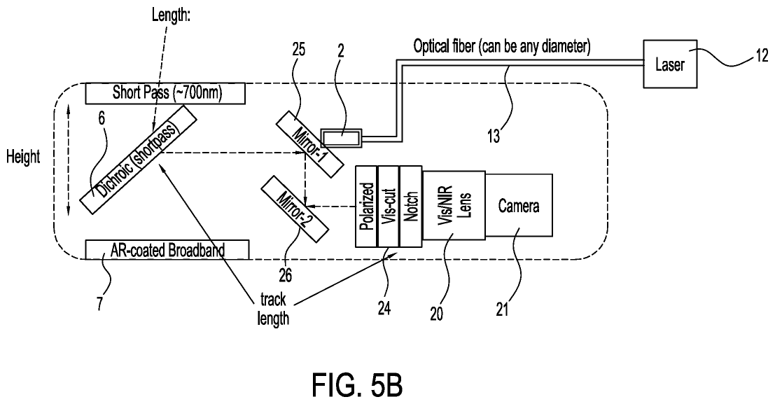

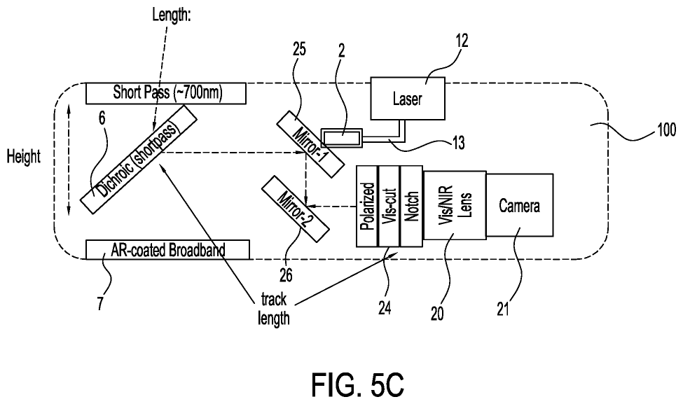

The imaging system can comprise a light source and an imaging system. In some embodiments, the light source is located internal to the imaging system 100, as shown in FIG. 5C. In some embodiments, the light source is adjacent to the imaging system. In some embodiments, the light source is located in close proximity to the imaging system. In some embodiments, the light source is located within about 10 mm from the imaging system.

Referring to FIGS. 4, 5A-5C, 6A-6B, 7A, and FIG. 16 in a particular embodiment, the light source 12 generates an excitation light beam, whereby the excitation light beam can have a wavelength in the ultraviolet, blue, visible, red, infrared, or NIR range as described herein. In this embodiment, the light source 12 can be coupled to an optical fiber 13. Alternatively, the light source can be directly coupled with a free space optic such as a mirror. The light from the optical fiber 13 can then be collimated using a collimator lens 17. In some embodiments the laser spectral characteristics correspond to the peak absorption value of the fluorophore.

After collimation, the light can be cleaned and its spectral bandwidth can be reduced using a band-pass filter, such as a laser clean up filter 16. The laser clean up filter 16 can be configured such that the excitation light spectrum is narrower at the notch filter. The notch filter can be used to block reflected excitation source light from the target. The laser cleanup filter 16 can comprise a full width half maximum that is less than a full width half maximum of the notch filter in order to inhibit cross talk between the excitation beam and the fluorescence beam emitted from the sample. In some embodiments, the laser clean up filter and the notch filter both determine the spectral bandwidth. For example, the spectrum of the excitation source and the specific clean up filter can be configured such that the spectral width of the excitation beam emitted through the clean-up filter is narrower than the spectral width of the excitation beam emitted through the width notch filter. The spectral width of the notch filter as disclosed herein can be a full width half maximum dimension of a beam transmitted through the filter. The clean-up filter can have a bandpass as described herein, depending on the excitation wavelength and fluorophore used. For example, in some embodiments, the clean-up filter has a bandpass of 15 nm (rejection of >4OD at 25 nm) depending on excitation wavelength and fluorophore used. In some embodiments, the laser energy is in the spectral bandwidth in the range of 5 nm with rest of the energy in wider spectral range up to but not limited to 15 nm.

In some embodiments, the laser cleanup filter narrows the bandwidth of the light source by about 1% to about 90%. In some embodiments, the laser cleanup filter narrows the bandwidth of the light source by about 1% to about 2%, about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 2% to about 5%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some embodiments, the laser cleanup filter narrows the bandwidth of the light source by about 1%, about 2%, about 5%, about 10%, about 20%, about 30%, about 40%, about 50%, about 60%, about 70%, about 80%, or about 90%. In some embodiments, the laser cleanup filter narrows the bandwidth of the light source by at least about 1%, about 2%, about 5%, about 10%, about 20%, about 30%, about 40%, about 50%, about 60%, about 70%, or about 80%. In some embodiments, the laser cleanup filter narrows the bandwidth of the light source by at most about 2%, about 5%, about 10%, about 20%, about 30%, about 40%, about 50%, about 60%, about 70%, about 80%, or about 90%.

In some embodiments, the laser cleanup filter narrows the bandwidth of the light source by about 1 nm to about 100 nm. In some embodiments, the laser cleanup filter narrows the bandwidth of the light source by about 1 nm to about 2 nm, about 1 nm to about 5 nm, about 1 nm to about 10 nm, about 1 nm to about 20 nm, about 1 nm to about 30 nm, about 1 nm to about 40 nm, about 1 nm to about 50 nm, about 1 nm to about 60 nm, about 1 nm to about 70 nm, about 1 nm to about 80 nm, about 1 nm to about 100 nm, about 2 nm to about 5 nm, about 2 nm to about 10 nm, about 2 nm to about 20 nm, about 2 nm to about 30 nm, about 2 nm to about 40 nm, about 2 nm to about 50 nm, about 2 nm to about 60 nm, about 2 nm to about 70 nm, about 2 nm to about 80 nm, about 2 nm to about 100 nm, about 5 nm to about 10 nm, about 5 nm to about 20 nm, about 5 nm to about 30 nm, about 5 nm to about 40 nm, about 5 nm to about 50 nm, about 5 nm to about 60 nm, about 5 nm to about 70 nm, about 5 nm to about 80 nm, about 5 nm to about 100 nm, about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 100 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 100 nm, about 40 nm to about 50 nm, about 40 nm to about 60 nm, about 40 nm to about 70 nm, about 40 nm to about 80 nm, about 40 nm to about 100 nm, about 50 nm to about 60 nm, about 50 nm to about 70 nm, about 50 nm to about 80 nm, about 50 nm to about 100 nm, about 60 nm to about 70 nm, about 60 nm to about 80 nm, about 60 nm to about 100 nm, about 70 nm to about 80 nm, about 70 nm to about 100 nm, or about 80 nm to about 100 nm. In some embodiments, the laser cleanup filter narrows the bandwidth of the light source by about 1 nm, about 2 nm, about 5 nm, about 10 nm, about 20 nm, about 30 nm, about 40 nm, about 50 nm, about 60 nm, about 70 nm, about 80 nm, or about 100 nm. In some embodiments, the laser cleanup filter narrows the bandwidth of the light source by at least about 1 nm, about 2 nm, about 5 nm, about 10 nm, about 20 nm, about 30 nm, about 40 nm, about 50 nm, about 60 nm, about 70 nm, or about 80 nm. In some embodiments, the laser cleanup filter narrows the bandwidth of the light source by at most about 2 nm, about 5 nm, about 10 nm, about 20 nm, about 30 nm, about 40 nm, about 50 nm, about 60 nm, about 70 nm, about 80 nm, or about 100 nm.

In some embodiments, the cleaned up light is then reflected by a dielectric mirror 15. The cleaned light can be reflected at an angle of about 60 degrees to about 120 degrees. The cleaned light can be reflected at an angle of about 90 degrees. The reflected light can then be diffused at calculated angle(s) through a hole in the NIR mirror 4 to match the cone of imaging light using a diffuser 14. In some embodiments, the diffuser also ensures that the excitation source's light is evenly distributed to produce a flat or relatively homogenous illumination profile on the target tissue. A nonlimiting example of the laser 12 is a BWT 8 W diode laser. Nonlimiting example of the optical fiber is a 105 um core optical fiber with a cladding of 125 μm, with a buffer of 250 μm and 0.22NA, and a length of 100 cm=/−10 cm. Nonlimiting example of the diffuser 14 is Thorlabs 20 degree circle engineered diffuser (RPC) #ED1-C20. Nonlimiting example of the collimator lens is Thorlabs A110™-B, f=6.24 mm, NA=0.40, Rochester Aspheric. Nonlimiting example of the laser clean-up filter is DiodeMax 785 Semrock-LD01-785/10-12.5. In some embodiments, the excitation light source includes one or more elements in the assembly 9, which can include one or more of but is not limited to collimator 17, clean up filter 16, dielectric mirror 15, and diffuser 14. In some embodiments, this cleaned up light is reflected at any angle, for example, between 45 degrees and 90 degrees, or between 90 degrees and 135 degrees, using a dielectric mirror. Moreover, in other embodiments although the cleaned up light is reflected at any arbitrary angle, with or without dielectric mirror.

Continuing to refer to exemplary FIG. 4, the dichroic shortpass filter 6, although it is shown that the light is coming from the “down direction” it is actually coming from perpendicular to the plane of the paper.

### Illumination and Excitation Sources

In some embodiments, the system comprises one or more excitation sources configured to generate an excitation beam to excite fluorescence tagged tissue and stimulate fluorescence in the region of tissue imaged. In some embodiments, the system comprises one or more illumination light sources configured to emit visible light in order to enable a user such as a surgeon to view the sample and non-fluorescent aspects.

The one or more illumination sources can act as an excitation light source. The one or more excitation sources can act as an illumination light source. At least one of the illumination source and the excitation source can comprise a visible light source. Visible light can be generated by a number of white light or visible light spectrum sources. At least one of the illumination source and the excitation source can comprise a broadband source, a narrowband laser, a wide band source, narrow-band light source, or any combination thereof. At least one of the illumination source and the excitation source can be an incoherent light or a coherent light.

At least one of the illumination source and the excitation source can comprise an incandescent lamp, a gas discharge lamp, a xenon lamp, an LED, a halogen lamp, or any combination thereof. The broadband source can emit NIR spectrum light. The wide band source can comprise a light emitting diode (LED) coupled to a notch filter.

At least one of the illumination source and the excitation source can be a visible, red, infrared (IR) near-infrared (NIR), ultraviolet, or blue light. The excitation light can comprise red light having a wavelength within a range from about 620 to 700 nm, red light having a wavelength of about 650 to about 700 nm, near infrared or infrared light having a wavelength of about 710 to about 800 nm, near infrared or infrared light having a wavelength of about 780 to about 850 nm, ultraviolet light having a wavelength of about 10 to 400 nm, ultraviolet light having a wavelength of about 200 to about 400 nm, blue light having a wavelength of about 380 to 460 nm, or blue light having a wavelength from about 400 to 450 nm.

At least one of the illumination source and the excitation source can be controlled by the imaging system, or be uncontrolled. The uncontrolled source can be, for example, a microscope light source, an ambient light source, or both. The excitation light source can comprise a laser or a wide band source (e.g., light emitting diode (LED)) coupled to a band pass filter.

In some embodiments, the excitation source has a wavelength of about 720, 750, 785, 790, 792, or 795 nm. In some embodiments, the excitation source has a wavelength in the infrared spectrum including light wavelengths the IR-A (about 800-1400 nm), IR-B (about 1400 nm-3 μm) and IR-C (about 3 μm-1 mm) spectrum. In some embodiments, the excitation source has a wavelength is in the near infrared (NIR) spectrum from 650 nm to 4000 nm, 700 nm to 3000 nm, 700-800 nm, 750 nm to 950 nm, 760 nm 825 nm, 775 nm to 795 nm, 780 nm to 795 nm, 785 nm to 795 nm, 780 nm to 790 nm, 785 nm to 792 nm, 790 nm to 795 nm, or any wavelength within any of these foregoing NIR ranges.

In some embodiments, the excitation source comprises a laser to cause the target (e.g., tissue tagged with fluorescence dye) to fluoresce and generate a fluorescence emission. The excitation source can alternate between on and off status. The visible light can or cannot be present to illuminate the target tissue in addition to the excitation source. In some embodiments, if there is a visible light source present in the system and method herein, it can have on and off status such that the light can be synchronously turned on/off with the excitation source. In some embodiments, external visible light such as from an operating microscope can be used. In some embodiments, the external light has an on and off status but is not synchronized with the excitation source's light. In other embodiments the external light source can be continuously on or continuously off.

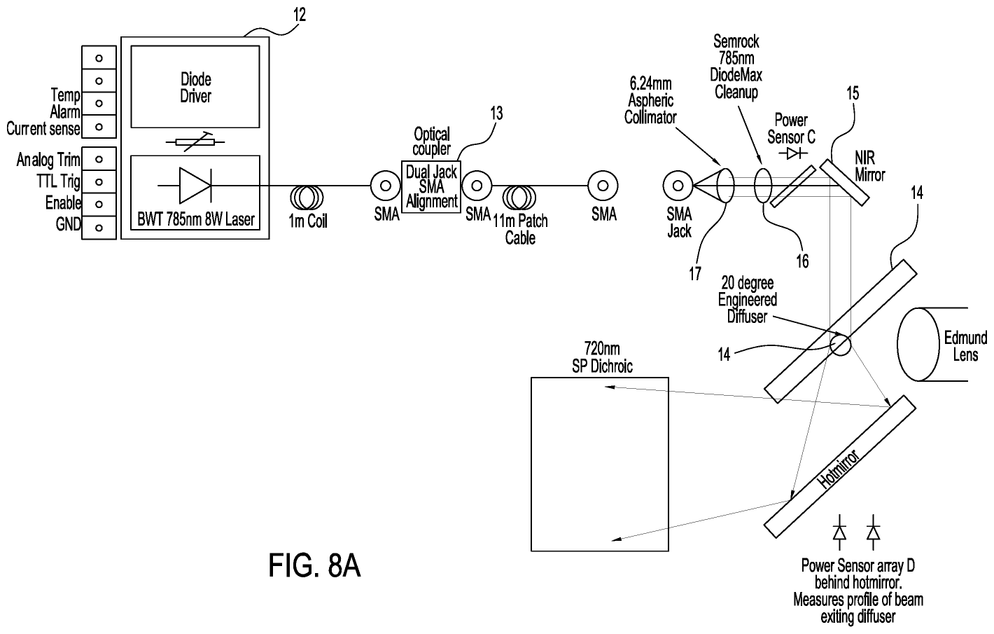

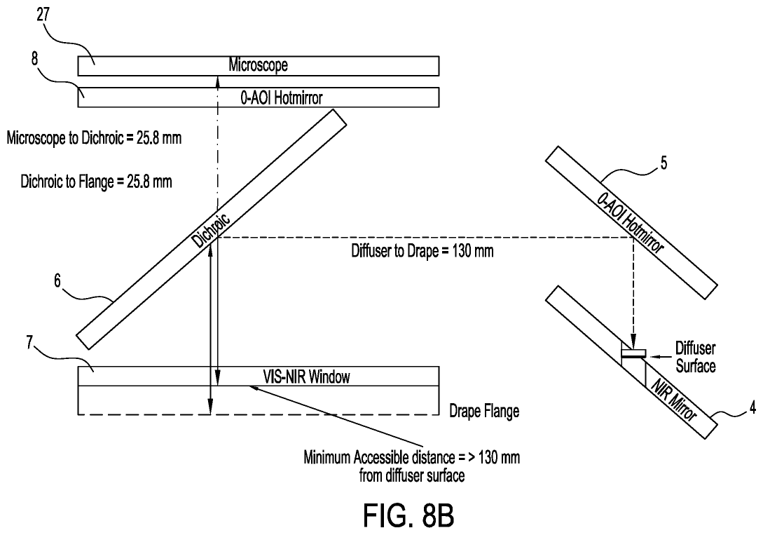

FIG. 8A shows an exemplary embodiment of the illumination opto-electrical system of the light source. In some embodiments, the systems and methods herein include one or more beam splitters, dichroic filters, dichroic mirrors, or use of the same. In some embodiments, the systems and methods include a primary dichroic mirror, and a secondary dichroic mirror. In some embodiments, the systems and methods include one or more shortpass dichroic mirrors and/or one or more longpass dichroic mirrors. In some embodiments, the beam splitters or dichroic mirrors, herein are configured to enable longpass-passing long wavelength while reflecting short wavelength (e.g. longpass filter or cold mirror) or shortpass—passing short wavelength while reflecting long wavelength (e.g., shortpass filter hot mirror). In some embodiments, the visible light herein is considered short wavelengths (e.g., shorter than 700 nm, or shorter than 780 nm) while the NIR or IR light are long wavelength (e.g., longer than 780 nm). In some embodiments, a mirror or filter herein includes filtering function (i.e., selective transmitting function) and/or or mirroring function (i.e., selective reflecting function).

The human eye can see color in the “visible light” spectrum from about 400 nm up to about 700 nm in light wavelength, although a person of ordinary skill in the art will recognize variations depending on the intensity of light used. The light provided to the user with eyepieces and the visible light imaging system will typically comprise wavelengths within this visible range. In some embodiments, the excitation beam comprises wavelengths shorter than at least some of the wavelengths transmitted with the eyepieces and used with the visible imaging system and detector, for example wavelengths ranging from 300 to 400 nm. In some embodiments, the excitation beam comprises wavelengths longer than at least some of the wavelengths transmitted with the eyepieces and used with the visible imaging system and detector, for example wavelengths shorter than about 650 nm. In some embodiments, the excitation wavelengths comprise frequencies greater than about 700 nm. For example, the dichroic mirror/filter can comprise a transition frequency of about-700 nm. (This optical element can also be referred to as 700 nm SP dichroic filter, for example.) By way of example, the shortpass (SP) dichroic filter can be configured to allow light with a wavelength of less than the transition frequency of about 700 to pass through the filter. This filter can be used to transmit more than 90% of the visible light, such that images seen by the user are substantially free of chromatic distortion, show very little dimming of the images seen through the eyepieces as compared with a microscope without this filter, which creates a better user experiences and allows a surgeon to better visualize the surgical field with decreased amounts of light that might otherwise interfere with the fluorescence measurement, in accordance with some embodiments. It is understood that the short pass filter can alternatively be a bandpass or notch filter. For example, one approximately “˜” 700 nm SP dichroic filter can comprise a FF720-SDi01 filter that has a transmission band Tavg=>90% for VIS (visible light), meaning that a 720 nm SP dichroic filter transmits >90% of visible light between 400 nm and 700 nm while reflecting >99% in the fluorescence emission band. The ˜700 nm SP dichroic filter allows most of the light (e.g., greater than 90%) shorter than about 700 nm through the dichroic filter, while reflecting almost all the light above about 700 nm. In some embodiments, these SP dichroic filters are very efficient in visible light filtering and are 99% efficient or greater with a transmission band Tavg=>99% for VIS (visible light) (e.g., when the incident light, e.g., visible light, or NIR light, on the filter is at a 45° angle). In other embodiments, the SP dichroic filters comprise >50%, >60%, >65%, >75%, >80%, >85%, >90%, >90.5%, >91%, >91.5%, >92%, >92.5%, >93%, >93.5%, >94%, >94.5%, >95%, >95.5%, >96%, >96.5%, >97%, >97.5%, >98%, >98.5%, >99%, >99.5%, >99.6%, >99.7%, >99.8%, or >99.9% efficiency or greater with a transmission band Tavg=>50%, >60%, >65%, >75%, >80%, >85%, >90%, >90.5%, >91%, >91.5%, >92%, >92.5%, >93%, >93.5%, >94%, >94.5%, >95%, >95.5%, >96%, >96.5%, >97%, >97.5%, >98%, >98.5%, >99%, >99.5%, >99.6%, >99.7%, >99.8%, or >99.9% for VIS (visible light). Moreover, in some embodiments, the ˜700 nm SP dichroic filter, while allowing transmission light to pass through at efficiencies comprising any of the foregoing, can also reflect >75%, >80%, >85%, >90%, >90.5%, >91%, >91.5%, >92%, >92.5%, >93%, >93.5%, >94%, >94.5%, >95%, >95.5%, >96%, >96.5%, >97%, >97.5%, >98%, >98.5%, >99%, >99.5%, >99.6%, >99.7%, >99.8%, or >99.9% in the fluorescence emission band.

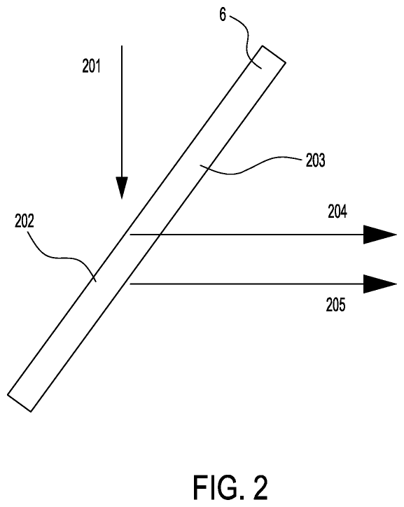

FIG. 2 shows an exemplary embodiment of a dichroic filter 6 having an anti-reflective coating 202 and a dichroic reflecting coating 203. As seen, in this embodiment, the dichroic filter 6 is placed so that the incident light 201 is at 45°. The incident light 201 can have a wavelength of less than about 700 nm. Light exiting from a back surface of the dichroic filter 204 having the anti-reflective coating 202 can have an intensity of less than about 1% of the intensity of the incident light 201 and a wavelength of less than about 700 nm. Light exiting from a front surface of the dichroic filter 205 having the dichroic reflecting coating 203 can have an intensity of greater than about 99% of the intensity of the incident light 201 and a wavelength of less than about 700 nm.

In some embodiments, the dichroic filter 6 is placed at 10°, 15°, 20°, 25°, 30°, 35°, 45°, 50°, 55°, 60°, 65°, 70°, or 75° relative to the incident visible/NIR or IR light path. In some embodiments, the reflection primarily happens on the front-coated surface 203 of the filter. In order to get better separation of light by wavelengths, the back side of the filter is coated with anti-reflection coating 202, thus further reducing reflection of the light <700 nm. In some embodiments, still a small amount (5-10%) of visible light (<about 700 nm) is reflected from the front as well as back of the filter. In some embodiments, 1%-5%, 3%-10%, 5%-12%, 10%-15%, up to 20% or less of visible light (<about 700 nm) is reflected from the front as well as back of the filter. In some embodiments, such a small amount, i.e., leaked visible light, is advantageous when used in the systems and methods herein for visible light imaging.

### Samples

The sample can comprise an ex vivo biological sample, such as a tissue sample. Alternatively, the sample can comprise in vivo tissue of a subject undergoing surgery.

The sample can include a marking dye. The marking dye can comprise an ultraviolet (UV) dye, a blue dye, or both. Exemplary UV and blue dyes for fluorophores include: ALEXA FLUOR 350 and AMCA dyes (e.g., AMCA-X Dyes), derivatives of 7-aminocoumarin dyes, dialkylaminocoumarin reactive versions of ALEXA FLUOR 350 dyes, ALEXA FLUOR 430 (and reactive UV dyes that absorb between 400 nm and 450 nm have appreciable fluorescence beyond 500 nm in aqueous solution), Marina Blue and Pacific Blue dyes (based on the 6,8-difluoro-7-hydroxycoumarin fluorophore), exhibit bright blue fluorescence emission near 460 nm, hydroxycoumarin and alkoxycoumarin derivatives, Zenon ALEXA FLUOR 350, Zenon ALEXA FLUOR 430 and Zenon Pacific Blue, succinimidyl ester of the Pacific Orange dye, Cascade Blue acetyl azide and other pyrene derivatives, ALEXA FLUOR 405 and its derivatives, pyrene succinimidyl esters, Cascade Yellow dye, PyMPO and pyridyloxazole derivatives, aminonaphthalene-based dyes and dansyl chlorides, dapoxyl dyes (e.g., Dapoxyl sulfonyl chloride, amine-reactive Dapoxyl succinimidyl ester, carboxylic acid-reactive Dapoxyl (2-aminoethyl)sulfonamide), bimane dyes (e.g., bimane mercaptoacetic acid) and its derivatives, NBD dyes and its derivatives, QsY 35 dyes and its derivatives, fluorescein and its derivatives. The marking dye can comprise an infrared dye, near infrared dye or both. Exemplary infrared and near infrared dyes for fluorophores include: DyLight-680, DyLight-750, VivoTag-750, DyLight-800, IRDye-800, VivoTag-680, Cy5.5, or an indocyanine green (ICG) and any derivative of the foregoing, cyanine dyes, acradine orange or yellow, ALEXA FLUORs and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GeiRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514.,., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are known and described in international patent application no. PCT/US2014/056177.

The marking dyes used for detection of a sample by the systems and methods herein can comprise one or more dyes, two or more, three, four five and up to ten or more such dyes in a given sample using any class of dye (e.g., ultraviolet (UV) dye, a blue dye, an infrared dye, or near infrared dye) in any combination.

### Cameras and Sensor

The system can comprise one or more imaging sensors to capture the fluorescence light and the visible light.

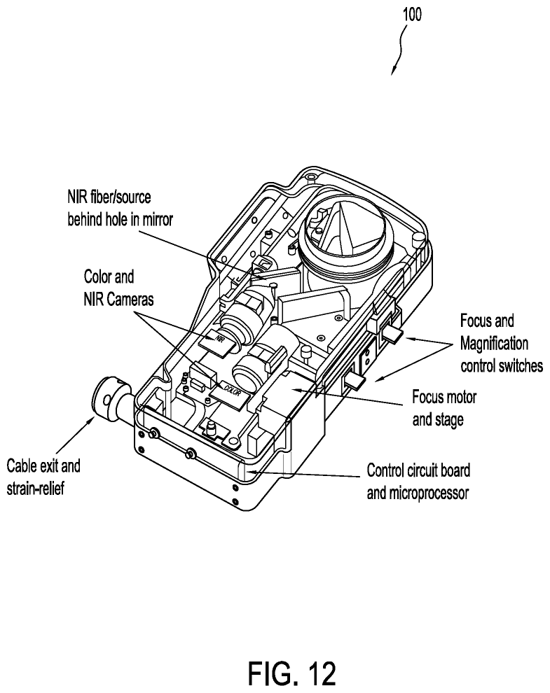

Referring to FIG. 12, in a particular embodiment, the imaging system 100 includes two separate cameras for substantially simultaneous acquisition of near infrared (NIR) fluorescence and visible light. In this embodiment, the imaging system can be attached to an operating microscope.

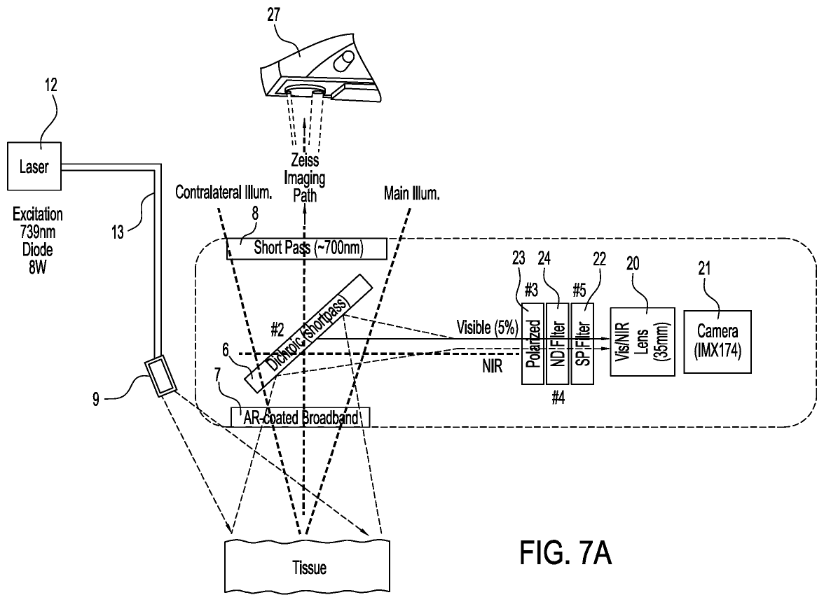

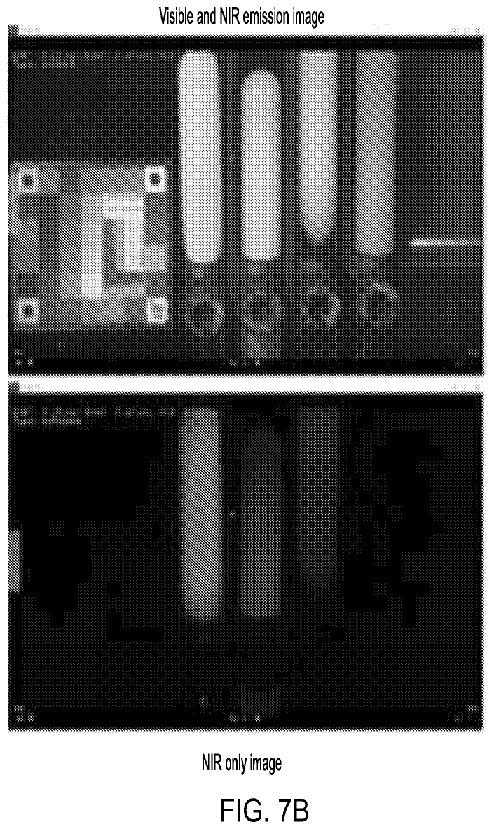

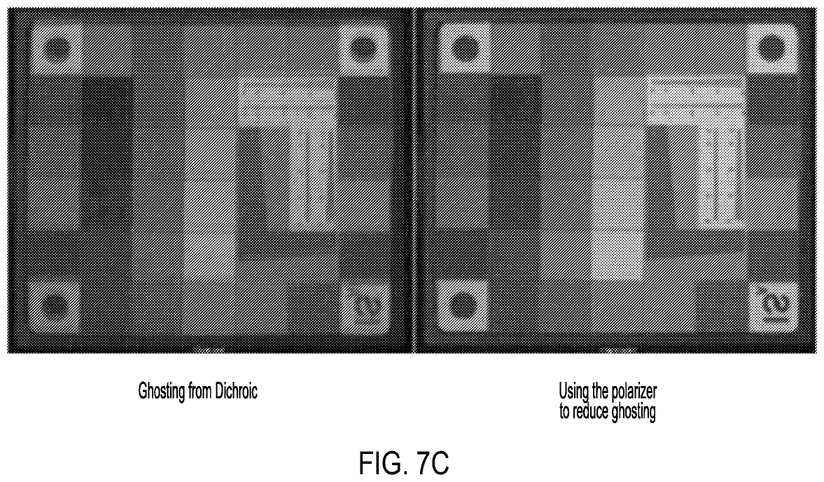

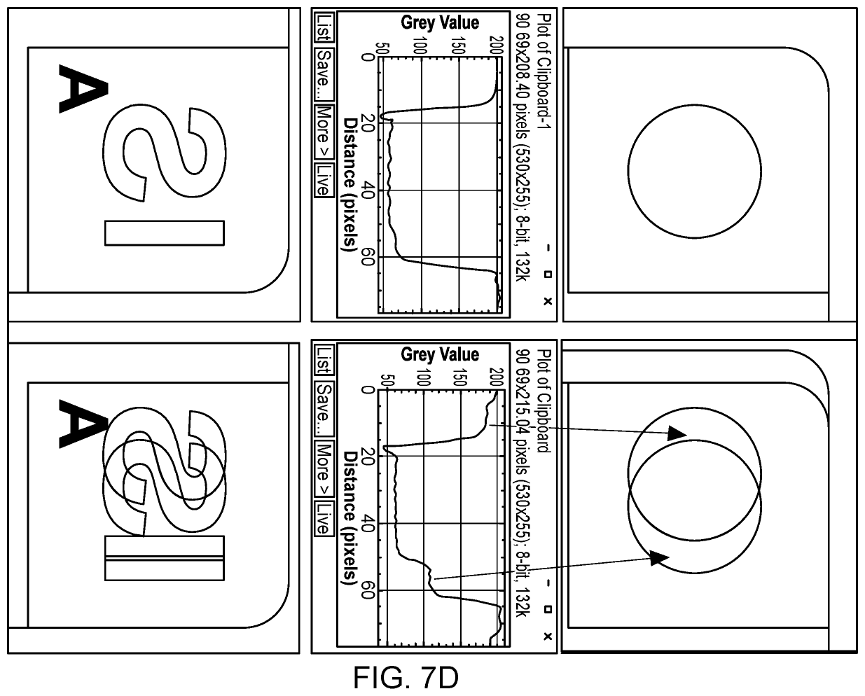

Referring to FIG. 7A, in a particular embodiment, the imaging system 100 includes a single camera for acquisition of near infrared (NIR) fluorescence and visible light. In this embodiment, the imaging system can be attached to an operating microscope. In some embodiments, the short pass filter only allows a wavelength of about 400 nm to about 700 nm to pass through. In some embodiments, the short pass filter has a safety for 793 nm leakage. In some embodiments, the short pass filter eliminates the NIR from the VIS camera image. In some embodiments, the short pass filter has a dichroic filter configured to remove the NIR from the uscope path. In some embodiments, the transmission is about 1% visible and about 99% NIR (about 800 mm to about 950 mm). In some embodiments the notch removes excitations having a wavelength of about 793 nm. In some embodiments, the VIS-cult and Notch filters are combined into a single filter. In some embodiments, the polarizer reduces ghosting and/or vis-cut OD blocking of the visual light. The filters (e.g., ND filter 24 and SP filter 22) as shown in FIG. 7A can be arranged in any alternative order.

In some embodiments, the systems and methods herein include one or more image sensors detectors, lenses, or cameras. In some embodiments, the detector herein includes one or more image sensors, lenses, and camera(s) herein. In some embodiments, the systems and methods herein are use a single camera, two cameras, or two or more cameras. In further embodiments, at least one camera is an infrared or NIR camera. In further embodiments, at least one camera is a VIS/NIR camera or a VIS/IR camera.

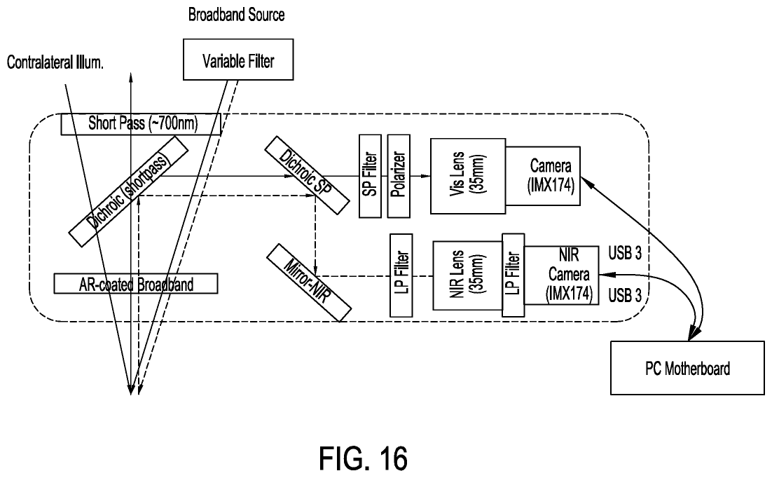

In some embodiments, the systems and methods herein is a single camera imaging system which only includes a VIS/NIR camera that is configured to sense both visible and NIR signals, as in FIGS. 5A-5B, 6A-6B, and 7A, and optionally in FIG. 4, FIG. 5C, and FIG. 16.

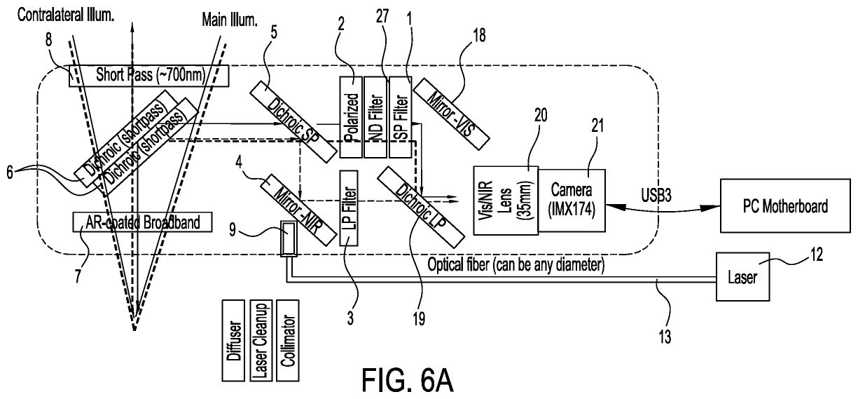

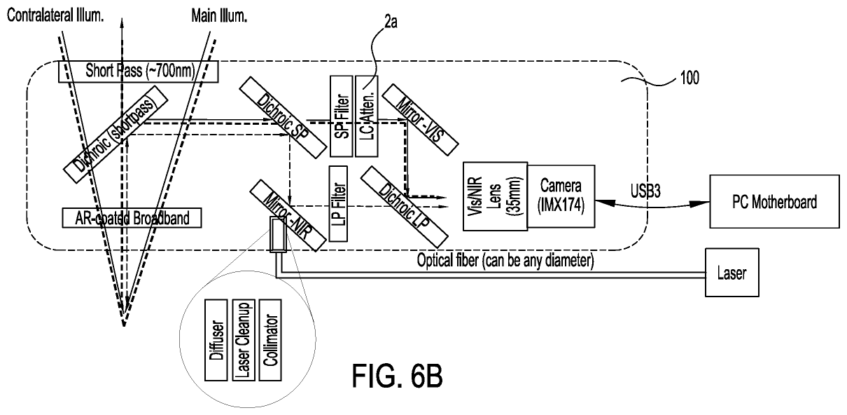

Referring to FIGS. 6A-6B, in a particular embodiment, the filtered visible light is reflected at a mirror 18 to a longpass dichroic filter 19 where it gets reflected again and combines with the filtered fluorescence signal to the single VIS/NIR lens 20 and camera 21 of the imaging system.

In some embodiments, two camera imaging systems herein advantageously allow one or more of: complete isolation of the VIS and NIR imaging paths, allowing filtering that is not wavelength or temporally dependent; reduction in temporal artifacts from visible light subtraction (e.g., with high ambient light, the dark frame can be of a significant higher brightness level relative to the infrared or NIR signal); shadow reduction from a dichroic filter without a corresponding loss in sensitivity in the infrared or NIR channel (e.g., the polarizer is only in the visible light path, not in the NIR light path); and there are no constraints on the brightness of the white light from the microscope, or other source of illumination of the surgical field.

In some embodiments, for a single camera design, a visible light filter, neutral density filter or LCD filter or any other optical element which passively or actively reduce the total amount of light passing through) e.g., 23 in FIG. 7A, is required to step down the intensity of the white light, while passing the NIR. In some embodiments, a shutter (e.g. LCD shutter, or ‘filter wheel,’ electronic variable optical attenuator (EVOA), an optical ‘chopper’, or a combination of polarizers can be synchronized to the excitation signal in order to selectively attenuate the visible light, but not the NIR. In some embodiments, a filter that physically moves can be used to selectively attenuate the visible light, but not the NIR. In some embodiments, such a filter sets the relative intensity of the VIS and infrared or NIR images and the dynamic range of the corresponding fluorescence signal.

In some embodiments, the two camera imaging system herein advantageously allows one or more of: a reduction in the required frame rate of the camera, allowing the use of smaller, longer data cables from the cameras; an increase in the bandwidth, since it isolates the frames and there are two data cables; a reduction in system cost by eliminating expensive frame grabber cards; allowing independent apertures on each of the VIS and infrared or NIR cameras for large depth of field on the VIS camera while not reducing the sensitivity in the NIR camera; not requiring the use of an apochromatic lens (corrected for infrared or NIR and VIS wavelengths to focus at the same imaging plane) and broadband coatings for optimal transmission in VIS and NIR as in the single camera imaging system.

In some embodiments, a single camera or a two-camera image system is selected at least partly based on specifics in applications.

In some embodiments, the two-camera imaging system herein advantageously allows different sensitivity (e.g., very high sensitivity for infrared or NIR and normal sensitivity for visible which can be useful in applications when the tissue can take up the dye but not in high concentration). Sensitivity range is defined by exposure time or frames per second (fps) displayed. A “normal” sensitivity can be about 25 fps display update, for example, when viewing tissues, samples or tumors with high uptake of a fluorescent compound or drug. High sensitivity can be a longer exposure as slow as 2 frames per second or any exposure longer than about 25 fps nearly capturing the autofluorecense in the tissues, or sample. FPS can be adjusted in real time to assess and implement the sensitivity needs for the application.

The two-camera image system herein can allow for varying the camera exposures for optimal sensitivity of the infrared or NIR images, without saturating the visible images. In some embodiments, the two-camera imaging system is used as a microscope attachment, exoscope, or surgical robot attachment or as a stand-alone imaging system for open field application(s).

In some embodiments, a single camera imaging system advantageously includes the ability to miniaturize the entire setup, e.g., for endoscopes. The single camera imaging system or the two-camera imaging system can be attached in front of a flexible or rigid endoscope (e.g., the optics and sensor of the endoscope are at the distal end towards the target while the body of the endoscope will carry the electrical signal from the sensor instead of optical as in normal endoscopes. In some embodiments, the single-camera or two-camera imaging systems herein is used in minimally invasive surgical approaches with endoscopes.

In some embodiments, the image sensors herein include a charge-coupled device (CCD) or complementary metal-oxide semiconductor (CMOS) image sensor.

A nonlimiting exemplary embodiment of the sensor used herein is the Sony IMX 174 CMOS chip in a Basler acA1920-155 camera. In this particular embodiment, the camera includes a 1/1.2 inch area sensor, a pixel size of about 5.86 μm, and a resolution of 1936×1216 (2.3 MP).

In some embodiments, the camera being used is a standard CMOS or CCD camera. These cameras are HD resolution, e.g., 1080 pixels, 4K, or higher pixel numbers. In some embodiments, the systems and methods here do not require specialized cameras such as EMCCD, ICCD etc. In some embodiments, the specialized cameras can be used to increase sensitivity, resolution, or other parameters associated with imaging. Table 1 shows information of exemplary embodiments of visible light and NIR cameras herein.

TABLE 1
Examples of Visible Light and NIR Cameras

| Property: | VIS: acA1920-155uc | NIR: acA1920-155um |
|---|---|---|
| Sensor size | 1936 × 1216 | |
| Sensor Type | Sony IMX174LLJ-C, Progressive Scan CMOS, | |
| | Global Shutter | |
| Optical sensor size | 1/1.2″ | |
| Effective sensor | 13.4 mm | |
| diagonal | | |
| Pixel Size (H × V) | 5.86 × 5.86 microns | |
| Max frame rate | 155 fps | |
| Data transport | USB 3.1, 5 Gbit/s | |
| Pixel formats | Mono 8 | Mono 8 |
| | Mono 12p | Bayer RG 8 |
| | Mono 12 | Bayer RG 12 |
| | | Bayer RG 12p |
| | | RGB 8 |
| | | BGR8 |
| | | YCbCr422_8 |
| Filter | None | Hot mirror |

| | |
|---|---|
| Size (L × W × H) | 48.2 × 29 × 29 mm |
| Weight | 80 g |
| Conformity | CE, UL in preparation, FCC |

In some embodiments, the systems and methods herein include one or more light sensor (e.g., photodiode, or other appropriate sensor). In some embodiments, the light sensors are configured for safety calculations and monitoring in the systems and methods. In some embodiments, light sensor(s) is located at the prism after the collimation lens, behind the dichroic SP 6, proximal end of excitation fiber and/or anywhere in the excitation path for total and relative power measurements. In some embodiments, two or any other number of photodiodes are located behind a hot mirror to monitor the shape of excitation source's illumination thereby ensuring diffuser performance.

In some embodiments, a one- or two-dimensional sensor array, or alternatively a CMOS array, is located behind a hot mirror to monitor the excitation source's illumination thereby ensuring diffuser performance.

### Optical Light Guides

The plurality of optics can be configured to illuminate the tissue and to collect the visible light and fluorescence light emitted therefrom. In some embodiment, the optical guide is not present and the laser travels in free space.

The plurality of optics can comprise a component selected from a list including but not limited to: a filter, an optical transmission mechanism, a lens, a mirror, and a diffuser. The filter can be configured to block light from the excitation source. The filter can comprise a band pass filter, a cleanup filter, or both. The band pass filter can be configured to control a wavelength of light. The cleanup filter can allow light with a certain wavelength and/or a certain angle of incidence to pass through. The cleanup filter can comprise a narrow-band bandpass filter. The mirror can comprise a dielectric mirror.

The optical transmission mechanism can comprise free space, or a light guide. The optical light guide can comprise an optical fiber, a fiber optic cable, a liquid light guide, a waveguide, a solid light guide, a plastic light guide, or any combination thereof. In some embodiments the optical fiber comprises silicate glass, plastic, quartz or any other material capable of transmitting excitation laser light. In some embodiments at least one of the plurality of optics comprises a coaxially light injection mechanism configured to provide additional coaxial light to the system. The coaxially light injection mechanism can comprise a through hole in one or more of the plurality of optics. It is understood that any type of optical transmission mechanism can be used in any of the embodiments of this system. The optical transmission mechanism can be configured to transmit infrared or near infrared light. The optical light can comprise a spliced or unspliced optical fiber. The diameter of the optical fiber can depend on the amount of power and the number of emitters in the excitation source, including the physics of collection optics.

In some embodiments, the optical fiber has a cross-sectional diameter of about 10 μm to about 1,000 μm. In some embodiments, the optical fiber has a cross-sectional diameter of about 10 μm to about 25 μm, about 10 μm to about 50 μm, about 10 μm to about 75 μm, about 10 μm to about 100 μm, about 10 μm to about 200 μm, about 10 μm to about 300 μm, about 10 μm to about 400 μm, about 10 μm to about 500 μm, about 10 μm to about 600 μm, about 10 μm to about 800 μm, about 10 μm to about 1,000 μm, about 25 μm to about 50 μm, about 25 μm to about 75 μm, about 25 μm to about 100 μm, about 25 μm to about 200 μm, about 25 μm to about 300 μm, about 25 μm to about 400 μm, about 25 μm to about 500 μm, about 25 μm to about 600 μm, about 25 μm to about 800 μm, about 25 μm to about 1,000 μm, about 50 μm to about 75 μm, about 50 μm to about 100 μm, about 50 μm to about 200 μm, about 50 μm to about 300 μm, about 50 μm to about 400 μm, about 50 μm to about 500 μm, about 50 μm to about 600 μm, about 50 μm to about 800 μm, about 50 μm to about 1,000 μm, about 75 μm to about 100 μm, about 75 μm to about 200 μm, about 75 μm to about 300 μm, about 75 μm to about 400 μm, about 75 μm to about 500 μm, about 75 μm to about 600 μm, about 75 μm to about 800 μm, about 75 μm to about 1,000 μm, about 100 μm to about 200 μm, about 100 μm to about 300 μm, about 100 μm to about 400 μm, about 100 μm to about 500 μm, about 100 μm to about 600 μm, about 100 μm to about 800 μm, about 100 μm to about 1,000 μm, about 200 μm to about 300 μm, about 200 μm to about 400 μm, about 200 μm to about 500 μm, about 200 μm to about 600 μm, about 200 μm to about 800 μm, about 200 μm to about 1,000 μm, about 300 μm to about 400 μm, about 300 μm to about 500 μm, about 300 μm to about 600 μm, about 300 μm to about 800 μm, about 300 μm to about 1,000 μm, about 400 μm to about 500 μm, about 400 μm to about 600 μm, about 400 μm to about 800 μm, about 400 μm to about 1,000 μm, about 500 μm to about 600 μm, about 500 μm to about 800 μm, about 500 μm to about 1,000 μm, about 600 μm to about 800 μm, about 600 μm to about 1,000 μm, or about 800 μm to about 1,000 μm. In some embodiments, the optical fiber has a cross-sectional diameter of about 10 μm, about 25 μm, about 50 μm, about 75 μm, about 100 μm, about 200 μm, about 300 μm, about 400 μm, about 500 μm, about 600 μm, about 800 μm, or about 1,000 μm. In some embodiments, the optical fiber has a cross-sectional diameter of at least about 10 μm, about 25 μm, about 50 μm, about 75 μm, about 100 μm, about 200 μm, about 300 μm, about 400 μm, about 500 μm, about 600 μm, or about 800 μm. In some embodiments, the optical fiber has a cross-sectional diameter of at most about 25 μm, about 50 μm, about 75 μm, about 100 μm, about 200 μm, about 300 μm, about 400 μm, about 500 μm, about 600 μm, about 800 μm, or about 1,000 μm.

In some embodiments, the optical light guide has a length of about 0.005 m to about 10 m. In some embodiments, the optical light guide has a length of about 0.005 m to about 0.01 m, about 0.005 m to about 0.05 m, about 0.005 m to about 0.1 m, about 0.005 m to about 0.5 m, about 0.005 m to about 1 m, about 0.005 m to about 2 m, about 0.005 m to about 3 m, about 0.005 m to about 4 m, about 0.005 m to about 6 m, about 0.005 m to about 8 m, about 0.005 m to about 10 m, about 0.01 m to about 0.05 m, about 0.01 m to about 0.1 m, about 0.01 m to about 0.5 m, about 0.01 m to about 1 m, about 0.01 m to about 2 m, about 0.01 m to about 3 m, about 0.01 m to about 4 m, about 0.01 m to about 6 m, about 0.01 m to about 8 m, about 0.01 m to about 10 m, about 0.05 m to about 0.1 m, about 0.05 m to about 0.5 m, about 0.05 m to about 1 m, about 0.05 m to about 2 m, about 0.05 m to about 3 m, about 0.05 m to about 4 m, about 0.05 m to about 6 m, about 0.05 m to about 8 m, about 0.05 m to about 10 m, about 0.1 m to about 0.5 m, about 0.1 m to about 1 m, about 0.1 m to about 2 m, about 0.1 m to about 3 m, about 0.1 m to about 4 m, about 0.1 m to about 6 m, about 0.1 m to about 8 m, about 0.1 m to about 10 m, about 0.5 m to about 1 m, about 0.5 m to about 2 m, about 0.5 m to about 3 m, about 0.5 m to about 4 m, about 0.5 m to about 6 m, about 0.5 m to about 8 m, about 0.5 m to about 10 m, about 1 m to about 2 m, about 1 m to about 3 m, about 1 m to about 4 m, about 1 m to about 6 m, about 1 m to about 8 m, about 1 m to about 10 m, about 2 m to about 3 m, about 2 m to about 4 m, about 2 m to about 6 m, about 2 m to about 8 m, about 2 m to about 10 m, about 3 m to about 4 m, about 3 m to about 6 m, about 3 m to about 8 m, about 3 m to about 10 m, about 4 m to about 6 m, about 4 m to about 8 m, about 4 m to about 10 m, about 6 m to about 8 m, about 6 m to about 10 m, or about 8 m to about 10 m. In some embodiments, the optical light guide has a length of about 0.005 m, about 0.01 m, about 0.05 m, about 0.1 m, about 0.5 m, about 1 m, about 2 m, about 3 m, about 4 m, about 6 m, about 8 m, or about 10 m. In some embodiments, the optical light guide has a length of at least about 0.005 m, about 0.01 m, about 0.05 m, about 0.1 m, about 0.5 m, about 1 m, about 2 m, about 3 m, about 4 m, about 6 m, or about 8 m. In some embodiments, the optical light guide has a length of at most about 0.01 m, about 0.05 m, about 0.1 m, about 0.5 m, about 1 m, about 2 m, about 3 m, about 4 m, about 6 m, about 8 m, or about 10 m. The length of the optical light guide can be measured as a minimum, average, or maximum distance between an input side and an output side of the optical light guide when the optical light guide is straightened.

In some embodiments, a laser module generates the excitation light, which is directed into an optical light guide. In some embodiments, an infrared source generates the excitation light, which is directed into an optical light guide. In some embodiments, a near-infrared source generates the excitation light, which is directed into an optical light guide.

In some embodiments, the diffuser has a diffuser surface. At least a portion of the diffuser surface can fit within a hole in the NIR mirror, for example, as shown in FIGS. 8A-8B. In this particular embodiment, one or more of the optical elements of the light source (e.g., collimator 17, clean up filter 16, dielectric mirror 15, and diffuser 14) can be located outside the hole of the NIR mirror. In other embodiments, one or more of the optical elements of the light source (e.g., collimator 17, clean up filter 16, dielectric mirror 15, and diffuser 14) can be located inside the hole of the NIR mirror. In other embodiments, one or more of the optical elements of the light source (e.g., collimator 17, clean up filter 16, dielectric mirror 15, and diffuser 14) can be located inside the surface of the NIR Mirror (e.g., mirror 4), or directly proximal to the mirror. In some embodiments, a distance from the diffuser to the drape is about 130 mm.

In some embodiments, the optical light guide includes an optical scaffold for introduction of the excitation light into the imaging system. In some embodiments, such a scaffold includes a hot mirror, dielectric mirror, silvered mirror, or the like, such as a NIR dielectric mirror 4. The excitation light can be inserted into the imaging system through a hole within the mirror.

In some embodiments, the system comprises one or more illumination sources. The one or more illumination sources can comprise an excitation light source such as a narrowband laser configured to generate an excitation beam to stimulate fluorescence in the region of tissue imaged. In some embodiments, the system comprises multiple excitation light sources. Alternatively or in combination, the excitation source can comprise a wide band source such as a light emitting diode (LED) coupled to a notch filter to generate the excitation light beam. The one or more illumination sources can comprise a visible light illumination source to illuminate the region of tissue imaged with visible light. A plurality of optics can be configured to illuminate the target and collect the visible light and fluorescence light. The plurality of optics can comprise filters to remove the light from the excitation source. The system can comprise one or more imaging sensors to capture the fluorescence light and the visible light. Moreover, a broadband source can be used as an illumination source. The broadband source can comprise a white light, an infrared light an incandescent lamp, a gas discharge lamp, a xenon lamp, an LED, or any combination thereof. The broadband source can emit NIR spectrum light for both illumination and excitation.

Referring to FIGS. 4 & 6A, in a particular embodiment, the target or sample is illuminated by the main illumination 12a and/or contra-later illumination 12b. The visible light from the target or sample is filtered by the primary dichroic shortpass filter 6, and only a small amount (i.e., leaked visible light), for example, 5-10% of the incident light at the shortpass filter 6 goes through a secondary dichroic filter 5 and reaches the visible lens 11a and camera 10a. In some embodiments, 1%-5%, 3%-10%, 5%-12%, 10%-15%, up to 20% or less of the incident light at the shortpass filter 6 goes through a secondary dichroic filter 5 and reaches the visible lens 11a and camera 10a. Nonlimiting exemplary embodiment of the visible camera is Basler acA1920-155uc. Nonlimiting exemplary embodiment of the NIR camera is acA1920-155 um. In some embodiments, 1%-5%, 3%-10%, 5%-12%, 10%-15%, up to 20% or less of the incident light at the shortpass filter 6 goes through a secondary dichroic filter 5 and is then filtered using a polarizer to remove shadows, neutral density filter (optional) and a short pass filter (to remove any traces of excitation light and fluorescence emission and gets further reflected by mirror FIG. 6A.

In some embodiments, the primary dichroic short pass filter 6 and the secondary dichroic filter 5 is any beam splitter, prism, filter, mirror, or other optical component that is configured to perform similar shortpass function as the dichroic filter.

Continue to refer to FIG. 4, in the same embodiment, almost all of the fluorescence light from the target or sample gets reflected by the primary dichroic shortpass filter 6 and then the secondary dichroic shortpass filter 5, thus separated from the majority of visible light at the primary dichroic filter and then separated from the leaked visible light at the secondary dichroic filter. In this embodiment, the fluorescence light gets reflected at the NIR mirror 4 and further filtered by a longpass filter 3 before it reaches the NIR lens 11b and NIR camera 10b. An additional NIR longpass filter 3.5 can be included between the NIR lens and the camera. In some embodiments, there is no additional NIR longpass filter between the NIR lens and the camera. In some embodiments, the aforementioned filters are infrared filters. Nonlimiting exemplary embodiment of the longpass filter 3 is Edmund UV/VIS cut imaging filter. Nonlimiting exemplary embodiment of the NIR longpass filter 3.5 is 808 nm longpass Semrock Edge Basic.

In some embodiments, the dichroic filter/mirror e.g., 5, 6, and/or 8 herein includes an angle of incidence (AOI). The angle of incidence is 0 degree, 45 degrees, or any other angles. In some embodiments, the angle of incidence is 10°, 15°, 20°, 25°, 30°, 35°, 45°, 50°, 55°, 60°, 65°, 70°, 75°, or any other angle. Nonlimiting exemplary embodiment of dichroic filter 5, 6 is Edmund 45AOI hot mirror and 720 nm SP filter from Semrock, FF720-SDi01-55x55, respectively.

In some embodiments, the dichroic filter 6 is a filter that is specifically configured to allow the specified amount of VIS reflection, with high surface quality to reduce reflections from the excitation source, and a short enough wavelength edge to allow reflection of the large cone-angle for the excitation that reflects at AOI of 45+/−10 degrees. In some embodiments, the dichroic filter allows the reflection of the large cone-angle for the excitation that reflects at an AOI of 10°, 15°, 20°, 25°, 30°, 35°, 45°, 50°, 55°, 60°, 65°, 70°, 75°, or any other angle +/−10 degrees. In some embodiments, the dichroic filter 6 causes shadows FIGS. 7C-7D (left in FIG. 7C and bottom panels in FIG. 7D) in the visible light image due to secondary reflection of the leaked visible light from back surface. This light has different polarization than the light emitted by first surface. This allows the use of polarizer to eliminate the secondary (shadow) images from the back surface. FIG. 7D show exploded views of top and bottom right corners of FIG. 7C. In this embodiment, shadows or ghosting is significantly reduced or even removed by the use of polarizer, LC attenuator, or other optical elements of similar functions.

In some embodiments, the dichroic filter 5 has various functions including but not limited to: reflecting the excitation beam; 2) reflecting the infrared or NIR fluorescence; 3) transmitting the visible image to the VIS camera. In some embodiments, this element is used for the splitting of the infrared or NIR and VIS paths.

FIG. 8B shows an exemplary embodiment of the path of light followed by the illumination from the light source. In this embodiment, the system includes a 0-AOI hot mirror 8 which is positioned between a 45 AOI hot mirror 6 and the microscope 27. In this embodiment, the hot mirror 8 is configured as a safety filter for reducing excitation from leaking into the microscope (e.g., 785 nm) and eliminates NIR illumination from the microscope light, of the tissue that will be mixed in the dark frame and requires subtraction from the actual NIR fluorescence. In some embodiments, the aforementioned functionalities are as applied to infrared light. In some embodiments, the aforementioned functionalities are as applied to excitation source's light in the infrared range or NIR range. In some embodiments, the aforementioned functionalities are as applied to an infrared source (e.g., a wide band source (e.g., light emitting diode (LED)) with a band pass filter) in the infrared range or NIR range.

In some embodiments, one or more of the dichroic filters or dichroic mirrors herein functions as a wavelength-specific beam splitter. In some embodiments, the dichroic filter herein is any optical element that is configured to perform passive wavelength-specific beam splitting or beam separation.

Referring to FIG. 4, in a particular embodiment, the NIR imaging path includes a longpass (LP) filter 3 (e.g., a dielectric-coated filter, with 0-degree angle of incidence) that reflects all light shorter in wavelength than 800 nm (greater than OD6 blocking for <800 nm). The primary function of this LP filter is to eliminate the excitation light reflected off the sample and thus enable the sensor to image the fluorescence signal. In some embodiments, with a single camera the long pass filter can be replaced by a notch filter (broader in spectral band than the band pass laser clean up filter) which will block only the excitation light while letting both the visible image as well as fluorescence image on the sensor.

In some embodiments, little or no fluorescence reaches the VIS camera, since >90% is reflected by the dichroic filter 5. The shortpass filter 1, in some embodiments, is to reduce excitation leakage into the VIS camera. The VIS camera can have an additional hot mirror placed in front of the sensor (not shown in FIG. 4).

In some embodiments, dichroic filter 5 is the primary splitting agent for the VIS and NIR imaging paths. In some embodiments, one or more SP and LP dielectric filters herein are primarily for attenuation of the excitation into the imaging lens.

In some embodiments, fluorescence signal from tissue is reflected by a dichroic shortpass filter while visible light passes through as if it is completely transparent. The reflected fluorescent light can be further reflected by a second shortpass dichroic before it is reflected again on a mirror and passes through a longpass filter unchanged (e.g., “unchanged” meaning with less than 1%, 2%, 3%, 4%, or 5% of attenuation while rejecting unwanted excitation) to reach the lens and sensor.

In some embodiments, 95% or even more of the visible light just passes through the dichroic shortpass filter, only a tiny amount is reflected (leaked by) the filter. The leaked visible light can pass unchanged through a secondary dichroic filter before a normal mirror reflects it. The visible light then can get reflected again by a dichroic longpass filter before it is received at the lens and imaging sensor, as shown in FIGS. 4, 6A-6B.

In some embodiments, a small portion of visible light is reflected from both the front and back surface of a dichroic mirror. Both the light rays travel a tiny bit different distance and thus can be focused on the sensor by the lens at a slight offset. Due to the thickness of the dichroic mirror, the back surface reflection has a longer optical path length, registering as an offset on the sensor, leading to a shadowing effect where the image appears doubled, as shown in FIGS. 7C-7D. In some embodiments, the light from the front surface is 90° rotated in polarization compared to light reflected from the back surface. Thus, this shadow effect can be eliminated using a polarizer 2 as shown in FIG. 6A. Alternatively, a liquid crystal attenuator 2a in FIG. 6B can be used for variable attenuation of the visible light. In this embodiment, in FIG. 6B, the LC attenuator polarizes (e.g., accepts linearly polarized light, rejecting other axis, as the LC is sandwiched between two polarizers) the incoming light, therefore reducing shadowing or ghosting. In some embodiments, the systems and methods herein include a polarizer positioned in front of or behind the LC for reducing shadowing or ghosting. In some embodiments, each member of crossed polarizers is placed on a side of the LC. In some embodiments, the systems and methods herein include no polarizer additional to the LC for reducing ghosting or shadowing. In some embodiments, the LC attenuator herein is inherently polarized and thus by controlling the polarization of LC, front or the back reflection of the dichroic mirror can be eliminated thereby removing shadowing or ghosting. But there can be a significant drawback in using a polarizer or a similar device in the systems and methods herein if the polarizer is in front of reflected near infrared light. In some embodiments, a polarizer or similar element reduces about 50% of the photons from the infrared fluorescence signal, which causes undesired fluorescence signal loss. In order to reduce shadows without affecting or reducing the infrared fluorescence signal, in some embodiment, the polarizer or similar device is used only on visible light but not the infrared or NIR light. In some embodiments, the positioning of the polarizer is in a separate image path from infrared or NIR signal, and in some embodiments the polarizer is behind the infrared or NIR light path, or placed in a separate image path from the NIR light path, in order to minimize shadows. In some embodiments, the polarizer is placed in front of the lens, camera or mirror without any additional optical elements there between. In some embodiments, the polarizer is placed at least behind the primary and/or the secondary dichroic filter/mirror. In some embodiments, the polarizer is placed in front of the lens, camera or mirror with only a notch filter and/or a VIS-Cut filter there between. Referring to FIGS. 4, 6A-6B, in a particular embodiment, the polarizer 2, attenuator 2a, or similar device is placed so that mixed visible and infrared light is split using a hot mirror 5 (which is a shortpass (SP) dichroic filter) in which the visible light (blue arrows) goes through filter 5 and then the polarizer 2 and onto a secondary visible light camera 11a, 10a or onto a mirror 18 with again reflects in back on a single sensor 21, with another longpass dichroic filter 19 which reflects the visible light on the sensor.

Referring to FIG. 5A, in one embodiment, the visible light directly reaches the VIS/NIR lens 20 and camera 21 after it is filtered by a polarizer 2 to remove shadows, an optional VIS-Cut filter (neutral density filter or LCD filter or any other optical element which passively or actively reduce the total amount of light passing through) 23 to selectively further attenuate the visible light if needed but not the IR or NIR light Alternatively, a synchronized ‘shutter’ (e.g. LCD, or ‘filter wheel’, or optical ‘chopper’, electronic variable optical attenuator (EVOA)) can be used to provide such attenuation. (e.g., 1% visible light transmission and about 100% NIR transmission in the range of 800-950 nm), and a notch filter 22 to remove light from the excitation source. The fluorescence light, in the same embodiment, after getting reflected at the primary dichroic mirror 6, is attenuated by the polarizer 2, transmitted through the VIS-Cut filter 23, and notch filter 22 to reach the single VIS/NIR camera 21. In some embodiments, the primary dichroic mirror 6 has a length of about 35 mm to about 40 mm, or about 23 mm to about 54 mm. In some embodiments, the primary dichroic mirror 6 has a height of about 29 mm to about 35 mm, or about 23 mm to about 38 mm. In some embodiments, a distance from the dichroic shortpass mirror to the VIS or NIS lens is less than about 50 mm. In some embodiments, a distance from the dichroic shortpass mirror to the VIS or NIS lens is less than about 1,000 mm.

Referring to FIGS. 5B-5C, a pair of mirrors 25, 26 can be used to allow coaxial illumination through a hole at mirror-1 25, and both the visible light and the fluorescence light are twice reflected at the pair of mirrors before they reach the polarizer 2. The VIS-Cut filter 24 is depicted.

In some embodiments, the systems and methods herein is a two-camera imaging system that are configured to sense either visible or NIR signals, separately, as in FIG. 4. In some embodiments, the systems and methods herein is a single-camera imaging system that are configured to sense both visible or NIR signals, as in FIGS. 6A & 6B. In some embodiments, a two-camera imaging system is capable of providing both infrared or NIR and visible light images when high levels of visible ambient light are present in the imaging environment (without adverse imaging artifacts or the use of a VIS-Cut filter). Nonlimiting examples of such high level of ambient light include: windows in the operating room, and lights in the operation room that are required to be ON during the imaging. In some embodiments, at least one of the components shown in FIG. 4 can be aligned perpendicular to the page in displayed orientation. In some embodiments, the NIR mirror 4 is a dielectric mirror. In some embodiments, the optical fiber 13 is bent. In some embodiments, the optical fiber 13 is unbent.

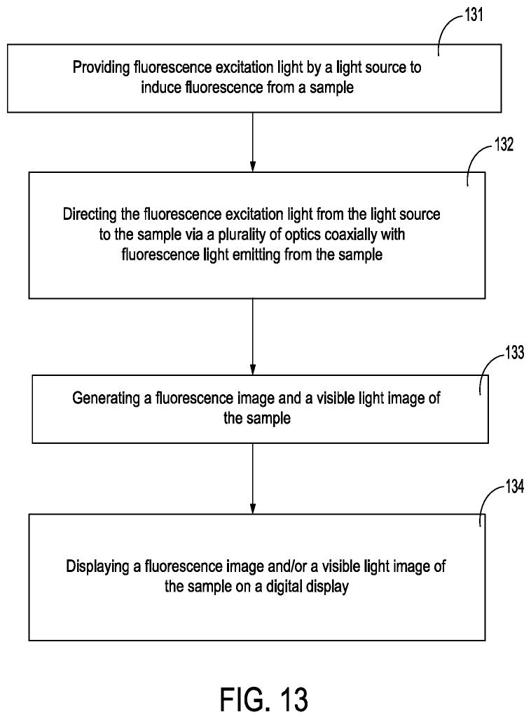

FIG. 13 shows an exemplary schematic diagram of one or more method steps for simultaneous visible light and fluorescence imaging using the imaging systems herein. In this particular embodiment, fluorescence excitation light, e.g., infrared light is provided by a light source to induce fluorescence from a sample 131. In some embodiments, the light source can be transmitted or “injected” through a hole in a dielectric mirror along the optical path of fluorescent light for NIR or IR imaging. In this embodiment, the infrared or NIR light from the light source is directed to the sample via a plurality of optics 132, the infrared light to the sample is substantially coaxial with fluorescence light received from the sample in order to decrease shadows in fluorescence image(s). The plurality of optics herein includes but is not limited to one or more of: a dichroic filter, a hot mirror, a beam splitter, a dielectric mirror, a polarizer, an attenuator, a notch filter, a neutral-density filter, a shortpass filter (e.g., wavelength shorter than 700 nm or 780 nm, or any wavelength between 700 nm or 780 nm), and a longpass filter (e.g., wavelength longer than 700 nm or 780 nm). In this embodiment, the imaging system herein generates a fluorescence image and a visible light image of the sample 133, the fluorescence image and the visible light image are not necessarily at the same frame rate. The fluorescence image(s) and the visible light image(s) can be processed by a processor to form a composite image. The composite image, the fluorescence image and/or the visible light image of the sample can be displayed to a user using a digital display 134.

FIGS. 4, 5A-5B, 6A-6B, and 7A show nonlimiting exemplary positions of the polarizer or attenuator with respect to the lens, camera and other elements of the image systems. In some embodiments, the polarizer or attenuator here can include one or more polarizer or attenuator that can be placed in other positions of the optical train.

In some embodiments, the systems and methods described herein include a notch filter, for example the notch filter (22) as shown in FIG. 5A. In some embodiments, the notch filter is in the optical path between a dichroic mirror and the imaging sensor. As shown in FIGS. 5A-5C, and 7A, optionally in FIG. 4, FIGS. 6A and 6B, and FIG. 16, in some embodiments, the notch filer is in between a primary dichroic mirror and the imaging sensor. In some embodiments, the notch filer is in between a polarizer and an imaging sensor. In some embodiments, the notch filter is configured to filter out at least a part of the excitation source's light (e.g., >90%, >90.5%, >91%, >91.5%, >92%, >92.5%, >93%, >93.5%, >94%, >94.5%, >95%, >95.5%, >96%, >96.5%, >97%, >97.5%, >98%, >98.5%, >99%, >99.5%, >99.6%, >99.7%, >99.8%, or >99.9% or more) and a lens can be used to focus the remaining fluorescence light on the sensor. In some embodiment the notch filter always has wider spectral band width that the band pass filter such as laser clean up filter. In some embodiments, the notch filter includes a spectrum width of about 20 nm at 0 degree AOI and 10 nm at 10 degree AOI. In some embodiments the notch filter is >OD3 for 770-800 nm for 0 degree AOI. In some embodiments, i.e., for non-zero AOL, the filter notch bandstop shifts to shorter wavelength whereby each 10 degrees it shifts by 5 nm. In some embodiments, the angle of incidence relative to the notch filter is 10°, 15°, 20°, 25°, 30°, 35°, 45°, 50°, 55°, 60°, 65°, 70°, 75°, 80°, 85°, or 90° or any other angle. It is understood that, depending on the AOL, the wavelength bandstop shifts accordingly.

In some embodiments, the working distance from an objective lens of the optical system to the tissue being imaged is less than 0.1 cm (1 mm), less than 0.2 cm (2 mm), less than 0.3 cm (3 mm), less than 0.4 cm (4 mm), less than 0.5 cm (5 mm), less than 0.6 cm (6 mm), less than 0.7 cm (7 mm), less than 0.8 cm (8 mm), less than 0.9 cm (9 mm), less than 1 cm, less than 2 cm, less than 3 cm, less than 4 cm, less than 5 cm, less than 6 cm, less than 7 cm, less than 8 cm, less than 9 cm, less than 10 cm, less than 20 cm, less than 30 cm, less than 40 cm, less than 50 cm, or more.

In some embodiments, the working distance is about 0.1 cm to about 50 cm. In some embodiments, the working distance is about 0.1 cm to about 0.2 cm, about 0.1 cm to about 0.5 cm, about 0.1 cm to about 0.7 cm, about 0.1 cm to about 0.9 cm, about 0.1 cm to about 1 cm, about 0.1 cm to about 5 cm, about 0.1 cm to about 10 cm, about 0.1 cm to about 20 cm, about 0.1 cm to about 30 cm, about 0.1 cm to about 40 cm, about 0.1 cm to about 50 cm, about 0.2 cm to about 0.5 cm, about 0.2 cm to about 0.7 cm, about 0.2 cm to about 0.9 cm, about 0.2 cm to about 1 cm, about 0.2 cm to about 5 cm, about 0.2 cm to about 10 cm, about 0.2 cm to about 20 cm, about 0.2 cm to about 30 cm, about 0.2 cm to about 40 cm, about 0.2 cm to about 50 cm, about 0.5 cm to about 0.7 cm, about 0.5 cm to about 0.9 cm, about 0.5 cm to about 1 cm, about 0.5 cm to about 5 cm, about 0.5 cm to about 10 cm, about 0.5 cm to about 20 cm, about 0.5 cm to about 30 cm, about 0.5 cm to about 40 cm, about 0.5 cm to about 50 cm, about 0.7 cm to about 0.9 cm, about 0.7 cm to about 1 cm, about 0.7 cm to about 5 cm, about 0.7 cm to about 10 cm, about 0.7 cm to about 20 cm, about 0.7 cm to about 30 cm, about 0.7 cm to about 40 cm, about 0.7 cm to about 50 cm, about 0.9 cm to about 1 cm, about 0.9 cm to about 5 cm, about 0.9 cm to about 10 cm, about 0.9 cm to about 20 cm, about 0.9 cm to about 30 cm, about 0.9 cm to about 40 cm, about 0.9 cm to about 50 cm, about 1 cm to about 5 cm, about 1 cm to about 10 cm, about 1 cm to about 20 cm, about 1 cm to about 30 cm, about 1 cm to about 40 cm, about 1 cm to about 50 cm, about 5 cm to about 10 cm, about 5 cm to about 20 cm, about 5 cm to about 30 cm, about 5 cm to about 40 cm, about 5 cm to about 50 cm, about 10 cm to about 20 cm, about 10 cm to about 30 cm, about 10 cm to about 40 cm, about 10 cm to about 50 cm, about 20 cm to about 30 cm, about 20 cm to about 40 cm, about 20 cm to about 50 cm, about 30 cm to about 40 cm, about 30 cm to about 50 cm, or about 40 cm to about 50 cm. In some embodiments, the working distance is about 0.1 cm, about 0.2 cm, about 0.5 cm, about 0.7 cm, about 0.9 cm, about 1 cm, about 5 cm, about 10 cm, about 20 cm, about 30 cm, about 40 cm, or about 50 cm. In some embodiments, the working distance is at least about 0.1 cm, about 0.2 cm, about 0.5 cm, about 0.7 cm, about 0.9 cm, about 1 cm, about 5 cm, about 10 cm, about 20 cm, about 30 cm, or about 40 cm. In some embodiments, the working distance is at most about 0.2 cm, about 0.5 cm, about 0.7 cm, about 0.9 cm, about 1 cm, about 5 cm, about 10 cm, about 20 cm, about 30 cm, about 40 cm, or about 50 cm.

### Coaxial Illumination

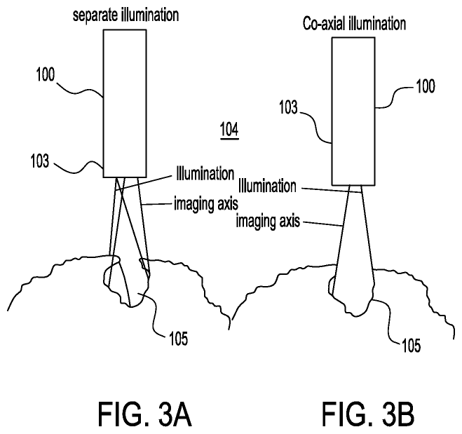

In some embodiments, as the illumination signal is injected though a hole in the mirror in the imaging path, the systems and methods herein enable coaxial illumination and light collection. Unlike prior imaging systems, the coaxial illumination of the devices herein enables visualization of organs, substructures of organs, targets, tissue and cells without casting a shadow on the sample being viewed. Avoiding shadows is beneficial to prevent obstruction from both the visible, infrared, and near infrared light within the images of the organs, substructures of organs, targets, tissue and cells. Further, such shadows can obstruct fluorescent signals from the tissue and cause false negatives. In some embodiments, the systems and methods herein utilize coaxial illumination to avoid this problem. FIG. 3A-B shows the coaxial illumination and imaging axes, in comparison to separate illumination and imaging axes. In this particular embodiment, coaxial illumination improves the visibility of the tissue by reducing shadows, thus false negatives (no fluorescence), thereby improving the imaging of a tissue cavity, organ, and substructure of organs, target, tissue or cell that is under observation by the system.

In some embodiments, the imaging axis of the microscope, the imaging axis of the imaging system herein, and the excitation axis are all coaxial with each other. In some embodiments, the image axis and the excitation axis share the same common axis.

In some embodiments, the imaging axis is aligned to the center of the right ocular axis or aligned to the left ocular axis, thus enabling a concentric field of view with the right ocular axis or the left ocular axis, for example. Alternatively, the light beam corresponding to excitation can extend toward the tissue from a location between the left and right objective lenses, and the imaging axis of the fluorescence camera can extend coaxially with the excitation axis from the tissue toward the sensor. The images may not necessarily comprise the same image size, and can comprise the same or different image sizes. The center point of each coaxial beam can be aligned so both beams are within an appropriate tolerance of each other so as to be considered coaxial as would be understood by one of ordinary skill in the art. In some embodiments, coaxial imaging as described herein corresponds to the excitation axis (e.g., visible or NIR/IR) substantially overlapping or being substantially parallel with the imaging axis of image sensors (e.g. of camera), or other imaging axis of the imaging systems disclosed herein such as the left and right eyepieces and objective lenses. The imaging axes can be configured for visible and/or fluorescence imaging such as NIR/IR light imaging. For example, systems disclosed herein can comprise: 1) an imaging axis for visible light corresponding to an image as seen by the user through an eyepiece of the microscope, 2) the fluorescent light imaging axis such as infrared or NIR light received from the sample, and 3) the excitation light beam axis directed to the sample, are all coaxial with each other (i.e., they share the same common axis, or at least within an appropriate tolerance as disclosed herein).

In some embodiments, substantially overlapping or parallel includes an intersecting angle between two axes to be less than 30 degrees, 20 degrees, 10 degrees, less than 5 degrees, less than 2 degrees, less than 1 degree, less than 0.1 degree, or less than 0.01 degree or about 0 degrees. Substantially overlapping can correspond to beams that are coaxial to within an acceptable tolerance of each other, e.g. to within 1 mm, 0.5 mm, 0.25 mm or 0.1 mm of each other. In some embodiments, substantially overlapping or parallel includes an intersecting angle between two axes to be less than 10 degrees, less than 5 degrees, less than 2 degrees, less than 1 degree, less than 0.1 degree, or less than 0.01 degree or about 0 degrees. The working distance from an objective lens of the optical system to the tissue being imaged can be within a range from about few millimeters (less than 1 cm) (e.g., endoscope) to 200-500 mm (e.g., microscope) or longer (e.g., open field imaging system).

In some embodiments, coaxial imaging does not include stereoscopic imaging. In some embodiments, coaxial imaging as disclosed herein includes overlap of two or more optical paths, at least one for illumination, and at least one other for imaging. Moreover, in some embodiments, two or more optical paths can be coaxially aligned to enable coaxial visualization of multiple infrared or near infrared wavelengths, for example from two or more fluorophores that home, target, migrate to, are retained by, accumulate in, and/or bind to, or are directed to an organ, organ substructure, tissue, target, cell or sample. In some embodiments, two or more, three or more, four or more, or five or more such paths are coaxially positioned. In some embodiments, the infrared or near infrared light is delivered to the sample along an infrared or near infrared optical path and the fluorescent light received from the sample is received along a fluorescence optical path and wherein the fluorescence optical path overlaps with the infrared optical path at a beam splitter. In some embodiments, the intersecting angle between two axes comprises no more than 10 degrees, no more than 5 degrees, no more than 2 degrees, no more than 1 degree, no more than 0.1 degree, or no more than 0.01 degree or about 0 degrees.

In some embodiments, coaxial imaging herein includes concentric fields of view (not necessarily the same image size, but the center point of imaging systems (e.g., microscope, imaging system, etc) are aligned). In a coaxial imaging system, there is no user perceptible parallax as the working distance changes. In a coaxial imaging system, the imaging shift due to variation in the accuracy of coaxiality does not exceed 5 mms at any working distance. In some embodiments, the imaging axis of the imaging system herein is aligned to the center of the right/left ocular axes, for example with reference to endoscopic applications.

### Eliminating Stray Light

Many current devices lack light isolation components to shield against room lighting such as fluorescent bulbs and tubes which can emit both visible and/or infrared wavelengths. Additionally, visual illumination by the device can interfere with fluorescence excitation. Further, as such devices lack components to characterize stray light, they must be used in a dark room to eliminate or diminish external light, stray light, ambient light, and continuous wave light. Light isolation, however, greatly improves image quality by reducing interference from non-visible wavelengths, visible wavelengths, infrared wavelengths, or any combination thereof.

In some embodiments, the systems and methods herein eliminate interference between visual and fluorescence lights through synchronization patterns thereof. Such synchronization can employ optimization of ON/OFF rates of the excitation light, or other system light control.

Alternatively or additionally, the systems herein can further comprise an attenuator comprising a shield, a hood, a sleeve, a light shroud, a baffle, or any combination thereof to block, filter or attenuate stray light. The physical attenuator can block, filter or attenuate such stray or ambient light to enhance the methods and systems of the disclosure. The attenuator can be external or affixed to the systems herein, including any of the systems described in FIGS. 4, 5, 6, 7, and 16

### Microscopes

In some embodiments, the imaging system and/or the imaging system herein is stereoscopic. In some embodiments, the imaging system and/or the imaging system herein is not stereoscopic. In some embodiments, the imaging system and/or the imaging system herein is surgical microscope, confocal microscope, fluorescence scope, exoscope, endoscope, or a surgical robot.

In some embodiments the systems herein are used alongside, in addition to, combined with, attached to, or integrated into an existing surgical microscope, confocal microscope, fluorescence scope, exoscope, endoscope, or surgical robot. In some embodiments, the microscope herein is stereoscopic. Such exemplary microscope, exoscope, endoscope can include one or more of the following: KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., RetinaVue 100, RetinaVue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPAL1 NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz. Moreover, in some embodiments, the imaging, diagnostic, detecting and therapeutic methods herein are performed using the systems described herein alongside, in addition to, combined with, attached to, or integrated into such an existing surgical microscope, confocal microscope, fluorescence scope, exoscope, endoscope, surgical robot, microscope, exoscope, or endoscope as described above.

Any additional surgical microscope, confocal microscope, fluorescence scope, exoscope, endoscope, or surgical robot systems can be used. The surgical microscope, confocal microscope, fluorescence scope, exoscope, endoscope, or surgical robot systems can be provided by, for example, Carl Zeiss A/G, Leica Microsystems, Leica Biosystems, Haag-Streit (5-1000 or 3-1000 systems), or Intuitive Surgical (e.g.: da Vinci surgical robot system), or any other manufacturer of such systems.

Combining or integrating a system herein into an existing surgical microscope, confocal microscope, fluorescence scope, exoscope, endoscope, or a surgical robot can be accomplished by: co-housing (in whole or in part), combining one or more aspect or component of the disclosed systems into the existing system, or integrating one or more aspect or component of the disclosed systems into the existing system. Such a combination can reduce shadowing or ghosting, utilize confocal improvements, enhance coaxial imaging, increase image clarity, optimize imaging, enable overlapping of optical paths, and improve surgical work flow, amongst other features of the systems and methods disclosed herein. Further such a combination or integration can utilize beam splitters, dichroic filters, dichroic mirrors, polarizers, attenuators, a lens shuttering, frame rate, or any other feature of the systems disclosed herein, or any combination thereof. Additionally such combinations or integrations can reduce leakiness (imperfection) of one or more filters, utilize ON/OFF rates of visible and fluorescent light sources, or both.

Further, the lighting external to the systems herein, e.g., from the microscope, can be very bright (e.g., ˜300W), which means that the difference between the intensity of visible light compared to the intensity of fluorescence emission can be substantial. In the embodiments with a single sensor, for example, as shown in FIG. 7A, this can be a disadvantage as the increased sensitivity settings such as higher gain of the sensor or longer exposure can lead to saturation of the light in visible spectrum, thus, such a very small leaked amount, can be advantageous for imaging using a high gain on a sensor (e.g., Sony IMX-174, 1/1.2″ sensor, and the like) quantum efficiency of >60%, dynamic range 73 dB.) to get a visible image. So as to fill around half of the dynamic range of the sensor. The imaging system described herein can use either one or two cameras, and records the leaked light in the visible range. In most optical applications, such dichroic filters and other types of band pass filters used as intended in a system are used to block 100% of light outside of the band pass range (e.g., here visible light) and not allow any leakiness of those blocked band widths through the filter. The point in using dichroic filters and other band pass filters in such systems is to only allow the light within the band pass through. However, as applied to some embodiments of the systems and methods herein, this leakiness (imperfection) of the filter is superior functionally, and used as an advantage to reduce the visible light entering the optical systems described.

In some embodiments, the optical light guide is a liquid light guide or other light guide. In some embodiments, the optical light guide couples to a lens which collimates the diverging output light from the fiber. The collimated light from the collimating lens can then pass through a band pass filter which can be a laser cleanup filter to further reduce the spectral bandwidth of the excitation source light. In some embodiments, the light is then diffused using a diffuser. This diffused light is then illuminated on the tissue in such a way as to match the field of view of the microscope and/or the field of view of the operating field.

In some embodiments, the diffuser is configured to match the illumination cone to the imaging field of view of the visible light (VIS), the imaging field of view of the near infrared (NIR) or infrared fluorescence, the microscope imaging field of view, or any combination thereof. In some embodiments, the hole in the NIR mirror 4 is sized, shaped, and/or positioned in to match the imaging axis of the visible light (VIS), the imaging axis of the near infrared (NIR) or infrared fluorescence, the microscope imaging axis, or any combination thereof. Such configurations ensure that the tissue which the surgeon is operating on through the operating microscope's ocular is completely illuminated and captured by the imaging system.

In some embodiments, the illumination path of the surgical microscope is independent of the dichroic filters, hot mirrors herein. In some embodiments, per FIG. 4 the diffuser, 14 determines the shape of the light beam exiting the hole in the mirror 4. The profile of the excitation light can be unaltered if outside the mirror. In other embodiments, the size of the hole is governed by the selection of a diffusers capable of diffusing the light in a cone of a certain angles. In other embodiments, the hole in the mirror is sized and positioned to achieve coaxial illumination, whereby the imaging axis is incident on the mirror angle and the illumination passes through the hole in the mirror. The hole size can be determined by one or more of: 1) a numerical aperture (NA) and/or core size of fiber which determines the final size of collimated beam incident on diffuser; 2) a feature size on diffuser (a minimum number of features (i.e., 1, 2, 3, 4, or 5 features or less, less than 10, 15, 20, 25, 30 features) can be illuminated to yield a good beam quality); 3) an f/# and focal length of the NIR lens—which can directly determine the maximum hole size so as to not visually obstruct the NIR imaging path and a corresponding reduction in the sensitivity as seen at the detector; or 4) a laser class level and maximum permissible exposure are based on the area of the retina for thermal hazard, where the smaller the beam on the diffuser, the smaller the area illuminated on the back of the retina and therefore the lower the laser power at the tissue for a given classification (e.g., such laser classification, for example in accordance with the ANSI Z136.1 Standard (Z136.1-2000) which assigns lasers into one of four broad hazard classes (1, 2, 3a, 3b and 4) depending on the potential for causing biological damage).

Per FIG. 4, the dichroic filter or dichroic mirror (5) can be positioned such that the visible and infrared images from the sample are coaxial, to allow the imaging system to superimpose the visible and infrared images on the display. In addition, the dichroic filter or dichroic mirror (6) can be positioned such that the imaging field of view of the microscope is coaxial with the visible and infrared images captured by the imaging system. Such alignment allows the imaging system to display the same field of view as is seen by the surgeon through the microscope.

In some embodiments, the white or visible light illumination from the microscope cannot be controlled or strobed by the imaging system herein. In some embodiments, the two-camera imaging system advantageously allows a non-multiplexed imaging path (e.g., NIR and visible images are not superimposed) in cases where they cannot be demultiplexed in time. In some embodiments, the imaging system allows strobing of the visible light for demultiplexing, thus a single camera system or a two-camera can both be used. In some embodiments, where control is available on the illumination and ambient light levels, a single camera imaging system can be used.

In some embodiments, the image system herein includes a hatch for servicing the imaging system (e.g., for allowing field reprogramming of the microcontroller firmware). In some embodiments, the hatch is located on the head of the imaging system. In some embodiments, the hatch is located on the back panel.

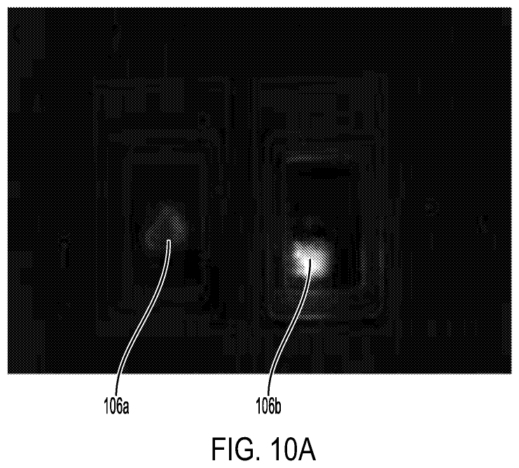

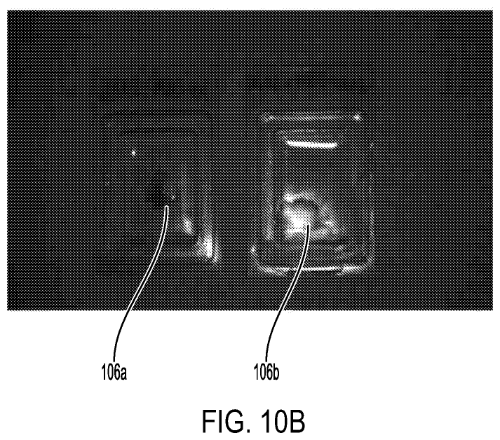

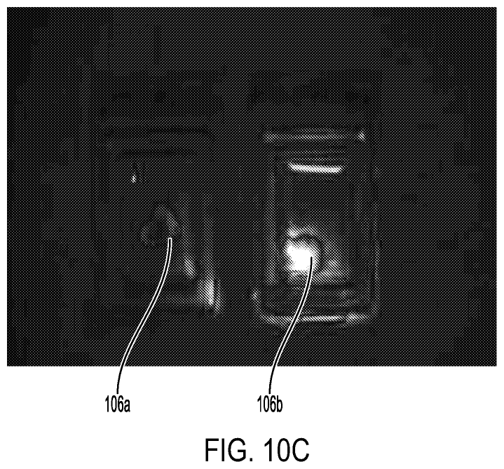

In some embodiments, the images, for example, FIGS. 1B and 10A-10C, generated by the systems and methods herein are displayed on a separate monitor. In some embodiments, the surgeon is able to select the type of images displayed: visible light image along with fluorescent image overlaid on top; or visible light image displayed in pseudo color, e.g., gray or red, and the fluorescent image displayed in different pseudo color, e.g., teal (blue+green) to achieve high contrast while maintaining the context of surrounding non-fluorescent tissue. In some embodiments, only visible or only fluorescent images can be displayed. In some embodiments, the images of different display types can be placed side by side for display. In some embodiments, the image display is not restricted to a monitor. In some embodiments, the images or videos can be just as easily displayed in surgeon's microscope, or augmented reality glasses, virtual reality glasses, or even used to display remotely for applications such as robotic surgery.

In some embodiments, if an infrared or a NIR frame is not ready, visible frames can take one or more previous NIR frame from the memory/buffer.

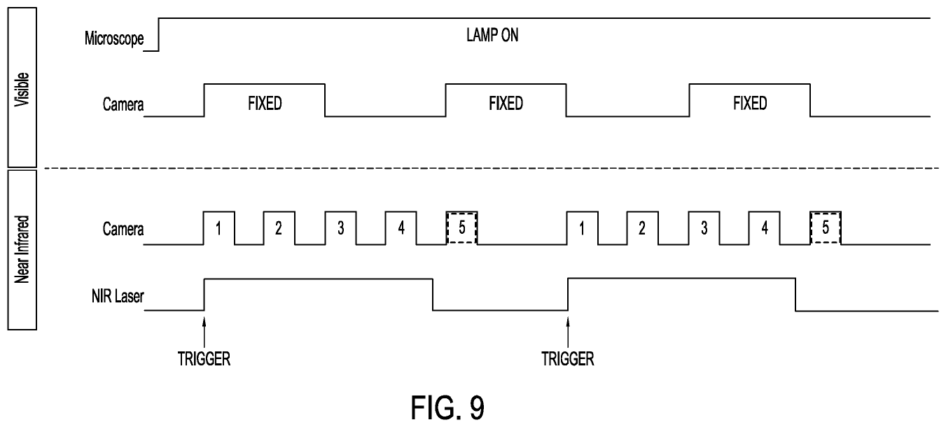

In a nonlimiting exemplary embodiment, the systems and methods herein include two cameras. On some embodiments, the system displays both visible and IR or NIR frame simultaneously even if the capture rate is not the same. In some embodiments, the infrared camera captures fluorescence light generated from the tissue when the tissue is excited by the excitation source's light. In some embodiments, the excitation source's light, as can be seen in FIG. 9, is not continuously “ON”. The excitation source's light can be turned on/off rapidly, or strobed either automatically or manually, using a digital processing device. In some embodiments, the excitation source's light can be modulated on/off using a mechanical means; e.g. a shutter or filter wheel, electronic variable optical attenuator (EVOA), or optical ‘chopper’, or a combination of polarizers. In some embodiments, in synchronization with the capture of each frame in the camera. The time when the excitation source is ON or OFF can be dynamically controlled and in real time. In an exemplary embodiment, the excitation source is ON for 1 to 10, 1-2, 1-4, 1-5, 1-6, 1-8, 1-20, 1-50, 1-60, 1-100 or any other frame ranges for NIR fames (i.e., frames captured by the infrared camera). The excitation light can be turned off for one of the above mentioned frames (dark frame). The dark frame when the excitation source is OFF, the sensor/camera captures all the light which is not from the tissue but is usually stray light in the operation room or other imaging environment. In some embodiments, the dark frame is subtracted from the all the NIR frames to remove the artifacts from the ambient or stray light. Afterward, in this particular embodiment, the all the first frames are added and displayed as a single frame. In some embodiments, such image frame processing (subtract and/or addition) herein provides the user a great control over the frame capture. In one exemplary embodiment, 4 frames of NIR image corresponds to 1 dark frame (FIG. 9). In other embodiment, any number of 1 or more NIR frames can be followed by 1 dark frame.

In some embodiments, the visible (VIS) and NIR excitation are provided by the same broadband source. FIG. 16 shows an alternate illumination pathway that is external to the imaging system. The system can comprise a broadband source an AR-coated broadband filter, a first shortpass filter, a second shortpass filter, a first shortpass filter, a second shortpass filter, a first filter, a second lowpass filter, a polarizer, a variable filter, a NIR mirror, a VIS lens, a NIR lens, a VIS sensor, a NIR sensor, and a PC motherboard.

As shown in in FIG. 6, light from the broadband source is directed through the window, is redirected by the first shortpass filter, is further redirected by the second shortpass filter and the NIR mirror, where it passes through the first lowpass filter, the NIR lens, the second lowpass filter and arrives at the NIR sensor. Additionally, contralateral illumination passes through the window, and to the first shortpass filter, wherein a portion of the contralateral illumination passes through the first shortpass filter to and through the first shortpass filter, and wherein a portion of the contralateral illumination is redirected by the first shortpass filter to the second shortpass filter to and through the second shortpass filter, the polarizer, and the VIS lens to arrive at the VIS sensor. The components of the system herein can be positioned and coupled using fasteners such as, for example, a screw, a nut and a bolt, clamps, vices, adhesives, bands, ties, or any combination thereof. The VIS sensor and the NIR sensor can then communicate with the PC motherboard based on the received light. The VIS sensor and the NIR sensor can communicate with the PC via a USB3 cable, a serial coax cable such as CoaXPress, an optical fiber, a serial cable, a USB C cable, parallel cable such as Camera Link, or any combination thereof.

The window can serve as a protection from dust particles and other foreign objects. The window can be fully transparent, and allow all or most wavelengths to pass. The window can have an anti-reflective coating. The window can have a filter. The filter can be a broadband filter. In some embodiments, the window is an AR-coated broadband filter. Additionally, this window can include notch filtering to reduce interference by other surrounding systems emitting wavelengths in the fluorescence band.

In some embodiments, at least one of the first shortpass filter and the second shortpass filter comprise a dichroic filter, an interference filter, a hot mirror, or dielectric mirror. Such filters can include dielectric mirrors, hot mirrors (a type a dielectric mirror), interference filters (e.g., a dichroic mirror or filter). In some embodiments, the system does not comprise the second shortpass filter. The first shortpass filter and the second shortpass filter can be congruent, whereas both filters allow the same band of wavelengths to pass. The first shortpass filter and the second shortpass filter can be incongruent, whereas both filters allow different bands of wavelengths to pass, whereby the different bands of wavelengths does or does not overlap. At least one of the first shortpass filter and the second shortpass filter can be custom made or can be selected from a commercially available filter. In some embodiments, the second shortpass filter includes power monitoring of the transmitted light behind the filter. One or more photodiodes or an array of photodiodes can be used to monitor beam shape and/or beam power. In other embodiments, the photodiodes are placed behind the hot mirror to enable transmission of light through the hot mirror.

In some embodiments, the polarizer comprises an absorptive polarizer, a beam-splitting polarizer, a birefringent polarizer, a Nicol prism, a Wollaston prism, a thin film polarizer, a wire-grid polarizer, a circular polarizer, a linear polarizer, or any combination thereof.

In some embodiments, the variable filter comprises an attenuator, a cross polarizer, filter wheel, a liquid crystal, an optical chopper, or a shutter or any other optical component that actively selects or transmits/blocks light of desired wavelengths. The variable filter selectively blocks or attenuates one wavelength band while transmitting another. The variable filter selectively blocks the visible light or dims it as required while not obscuring the NIR fluorescent signal. In some embodiment, the system does not comprise a variable filter.

In some embodiments, the NIR mirror comprises a dielectric mirror, a silver mirror, a gold mirror, an aluminum mirror, a hot mirror, or any combination thereof. The NIR mirror can comprise a dichroic mirror. The NIR mirror can comprise a coated mirror. The NIR mirror can comprise a hole to allow transmission of a laser from behind the NIR mirror. The NIR mirror can comprise a filter which reflects the fluorescence signal while transmitting the excitation wavelength(s), eliminating the physical hole in the optic. Additionally, the NIR mirror can comprise different coatings applied to different areas of the optic that optimize the area of reflection for the fluorescence signal while minimizing the area required for the “hole” that transmits the excitation wavelength(s). The small area for transmission is optimized for maximum transmission at one or more wavelengths while still allowing substantial reflection in the fluorescence band.

In some embodiments, at least one of the VIS lens and the NIR lens comprises a fixed focal length lens. At least one of the VIS lens and the NIR lens can have a focal length of about 10 mm to about 70 mm. In some embodiments, at least one of the VIS lens and the NIR lens comprises a 35 mm lens. Alternatively, at least one of the VIS lens and the NIR lens comprises a variable focal length. The size of the lens can directly correlate with the field of view of the system. The size of the lens can also determine an optimal size of the sensor. At least one of the VIS lens and the NIR lens can have a fixed F-number. Alternatively, at least one of the VIS lens and the NIR lens can have a variable F-number. The VIS lens and the NIR lens can have the same F-number. The VIS lens and the NIR lens can have different F-numbers. The VIS lens can have a greater F-number than the NIR lens. The NIR lens can have a greater F-number than the VIS lens. At least one of the VIS lens and the NIR lens can have an F-number of about 0.5 to about 11. In one exemplary embodiment, the VIS lens has an F-number of about 5.6 and the NIR lens has an F-number of about 1.65. In some cases, higher F-numbers enable higher image quality. In some cases, lower F-numbers enable higher image quality, depending on the applicability of the higher or lower F-number to the VIS or NIR lens, respectively. Unique f/#'s of NIR and VIS lenses can enable system offsets and optimization while maintaining focus. Anti-reflection coatings on the NIR and VIS lenses can be of the same broadband coating or can be individually optimized for NIR or VIS transmission. Optionally, both NIR and VIS lenses can be color corrected specifically for VIS and NIR, respectfully, or can be optimized for both VIS and NIR correction, reducing volume and cost.

In some embodiments, at least one of the VIS sensor and the NIR sensor comprises a visible sensor, a Complementary Metal Oxide Semiconductor (CMOS) sensor, or a Charge-Coupled Device (CCD) sensor. In some embodiments, at least one of the VIS sensor and the NIR sensor comprises an IMX174 sensor, a CMV2000 sensor, or an IMX134 sensor, high-resolution back plane sensor, or cell phone sensor. In some embodiments, at least one of the VIS sensor and the NIR sensor comprise a component within a commercially available camera. The pixel size and form factor of the sensor can be determined by the optical volume and the field-of-view required by the system. The pixel size and form factor of the sensor can be driven by system design specifications. Other embodiments can include any CCD or CMOS sensor, either operating as a complete camera or at the board level, integrated at the imaging station or prior to data transmission. Such processing can be formed at the imaging head via FPGA or by other means. The VIS camera can also include a Bayer filter mosaic or other color filter array to decode the RGB color information. Additionally, the color filter array can include the fluorescent band(s) for additional encoding beyond the pixel sensor array. Other examples of sensors can include back illuminated sensors, multiple sensor arrays (with or without filter arrays, e.g. monochrome), or cooled arrays. In some cases, the NIR sensor is a monochrome sensor. In some cases, the NIR sensor has a color filter array. Additional designs can include a filter array that selects different fluorescent band(s) or reduces interference from other emitting devices. Additionally, certain pixels can be filtered for either alignment to the VIS camera, enhancing resolution, and decoding spectral information.

In some embodiments, the PC motherboard comprises a commercially available PC motherboard. In one example, the commercially available is a PC ASUS ROG STRiX Z370-G micro-ATX motherboard, or an MSI Pro Solution Intel 170A LGA 1151 ATX motherboard.

In some embodiments, the broadband source emits visible through NIR spectrum is a Xenon lamp, a Xenon bulb, an LED light, a laser, a halogen lamp, a halogen bulb, sunlight, fluorescent lighting, or any combination thereof. The broadband source should be configured to provide balanced white light and should have sufficient power in the absorption band of the fluorophore to emit detectable fluorescence. In some instances, the broadband source is unfiltered. In some instances, the broadband source is non-blocked. The broadband light source can be naked, unhindered or non-controlled. In some cases, the broadband light source does not contain a shutter or a filter. Any of the systems and methods of the present disclosure can be used with such a broadband source, including, for example, the systems shown in FIGS. 4, 5, 6, 7 and 16. In other embodiments, the broadband source is filtered or shuttered or otherwise the input/output from the source is synchronized to capture various images. For example, the optical components in a filter or shutter ensure that the resultant VIS and NIR illumination is coaxial and within the same field of view. Any of the systems and methods of the present disclosure can be used with such a filtered or shuttered broadband source, including, for example, the systems shown in FIGS. 4, 5, 6, 7 and 16.

In some embodiments, such filtered or shuttered broadband sources can include a filter, a filter wheel, an electronic variable optical attenuator (EVOA), an optical ‘chopper’, aa polarizing shutter, modulator. Such filtering or shuttering enables passages of only certain wavelengths of light from the broadband source. Such filtering or shuttering can code image frames as either: 1) NIR only, where no visible light is emitted but non-visible light in the absorption band is passed, 2) visible only, with minimal inside the absorption band, or 3) stray or ambient only (shutter or “off”). In such embodiments, the light source can be external to the imaging system. In such embodiments, the light source can be, within an operating microscope. In such embodiments, the light source can be synchronized with the imaging system sync OUT, the light source sync IN, the imaging system sync IN, the light source sync OUT, or any combination thereof. In some embodiments, the synchronization between the filtered light and camera frame capture can comprise a master/slave relationship. In such cases, the light source can act as a master based on a filter in front of the light source. In such cases, the light source can act as a master based on a shutter state (e.g., ON/OFF, sync IN/OUT, etc.). In such cases, the light source can send signal to camera to start and stop frame capture. Alternatively, per the illumination pattern in FIG. 9, each frame captured by the camera can be communicated to the light source/filter/shutter via a protocol. The protocol can comprise TTL (Transistor Transistor Logic). This arrangement can also be implemented in the optical designs shown in FIGS. 4-6 and 7. This arrangement can be further implemented with respect to the placement of the illumination path axis shown in FIG. 16. In general, the visible and fluorescence images can be captured by many acquisition schemes, including a 1-camera or a 2-camera scheme.

In other embodiments, the VIS and NIR excitation is provided by a gas discharge lamp, a Xenon lamp, an LED, a LASER, or any combination thereof. In some instances, such broad excitation source is unfiltered and non-blocked so that the broadband excitation source is naked, unhindered or non-controlled (i.e., does not contain a shutter or filter). Any of the systems and methods of the present disclosure can be used with such a broadband source, including, for example, the systems shown in FIGS. 4, 5, 6, 7 and 16.

In some embodiments, they system further comprises a filter, a bandpass-filter, a filter wheel, an electronic variable optical attenuator (EVOA), an optical ‘chopper’, aa polarizer shutter, aa modulator, or any combination thereof to selectively filter VIS and NIR excitation wavelengths from the broadband source. For example, a filter wheel might have a short-pass filter, a long-pass filter, or both, wherein the short-pass filter allows visible illumination to pass while blocking IR wavelengths. Alternatively, the long-pass filter can allow IR wavelengths to pass while blocking visible wavelengths. Moreover, a short-pass filter can be used to block IR light in conjunction with a neutral density (ND) filter, to allow both allow both VIS and NIR to pass from the broadband excitation source. Any of the systems and methods of the present disclosure can be used with such a broadband excitation source, including, for example, the systems shown in FIGS. 4, 5, 6, 7 and 16. In some cases, all VIS and NIR excitation wavelengths can be blocked where the system employs a single-camera which cannot decipher NIR and VIS channels. Blocking all VIS and NIR excitation wavelengths can cause a light flickering that can distract the surgeon. In some embodiments, the system does not comprise a filter, a sync to the light/camera, or both. In such cases, stray light can be emitted by the system.

The broadband source can be used “as is” or as a shuttered or filtered broadband source depending on the source of fluorophore or tissue or cells being detected. The illumination optics which form the beam or path of detection can be optimized or selected based on the field of view (FOV) of the microscope

In some embodiments, the system further comprises an imaging head strain relief. The imaging head strain relief can be attached to the imaging head, a cable of the imaging head, or both. The imaging head strain relief can comprise a two-part component. The imaging head strain relief can comprise a clamp over an existing terminated cable during manufacture of the imaging head. The imaging head strain relief can comprise a sleeve over an existing terminated cable during manufacture of the imaging head. The imaging head strain relief can be 3D printed. The imaging head strain relief can comprise a commercially available strain relief. A sleeve around the imaging head cable can be employed to increase the grip of a commercial or custom strain relief. The sleeve can be made of rubber, silicone, plastic, wood, carbon fiber, fiberglass, thermoplastic elastomer, fabric, other polymer, or any combination thereof.

The imaging head strain relief can further comprise a stop configured to prevent the imaging head strain relief from translating along the imaging head cable. Alternatively, the imaging head cable can comprise an integrated strain relieve. The imaging head cable can have a set flex rating. The stop can comprise a grommet, a screw, a tie, a clamp, a string, an adhesive, an O-ring, or any combination thereof. The imaging head strain relief can be configured to prevent, minimize or prevent and minimize binding against the microscope's cable during imaging head translation, microscope translation, or both. The imaging head strain relief can be configured to allow and limit twisting of the image head cable prevent to prevent cable damage and increase component lifetime. The internal surface of the strain relief can be smooth so as to not puncture the cables. Auto-balance of the scope head can accommodate the additional weight of the imaging head strain relief.

USB data from one or more of the cameras can be transmitted using optical serial communication rather than passive or active copper. Optical serial communication generally allows for higher flexibility and longer cable lengths. In further embodiments, such cables can enable electrical transmission, optical transmission, or both. In addition, passive cables with right angle and high-flex for focus stage can be included. The imaging head can comprise a locking key. The locking key can be configured to securely lock the imaging head onto the microscope. The locking key can be configured to securely lock the imaging head onto the microscope without requiring any tools. The locking keys can be permanently fixed via a lanyard to the imaging head to prevent fixing the head to the scope without locking it in place. Stray light shroud or baffle can be used between camera sensor and lens assembly: the optical system is focused by moving the camera sensor relative to the lens (fixed). This requires an open gap between the sensor and lens which is particularly sensitive to any stray light in the imaging head enclosure. A simple concentric tube design was constructed where one tube screws onto the camera C-mount and the other tube onto the lens support. The surfaces are painted with highly absorptive paint and overlap even when the sensor is at maximum extent of the focus range. Other embodiments can include a shield, hood, sleeve, light shroud, baffle or other physical attenuator to block, filter or attenuate such stray or ambient light to enhance the methods and systems of the disclosure. Such shield, hood, sleeve, light shroud, baffle or other physical attenuator can be external or affixes to the systems of the disclosure.

Stray light can be inadvertently admitted into the imaging head enclosure through a gap between the sensor and lens necessary for focusing the system by moving the camera sensor relative to the fixed lens. For example, any of the systems described in FIGS. 4, 5, 6, 7, and 16 and throughout the disclosure can be used as described above or throughout the disclosure to eliminate the problems with stray light or ambient light. As such, the system can further comprise a light shroud between the camera sensor and lens assembly. The light shroud can comprise a tray, a cover, a baffle, a sleeve, a hood, or any combination thereof. The light shroud can block, filter or attenuate such stray or ambient light to enhance the methods and systems of the disclosure. The light shroud can be external or be affixed to the systems of the disclosure. The light shroud can be internal or be integrated within the systems of the disclosure. In some embodiments, the light shroud comprises a first tube and a second tube, wherein the first tube attaches to the camera, and wherein the second tube attaches to the lens support. The first tube and the second tube can be concentric. The first tube and the second tube can overlap when the sensor is at maximum extent of the focus range. The light shroud can attach to the camera via the c-mound of the camera. The light shroud can attach to the first tube, the second tube, or both via a fastener. The fastener can comprise an adhesive, a screw, a bolt, a nut, a clamp, a tie, or any combination thereof. The surfaces of the light shroud can be painted with or be formed of a highly absorptive paint and. Any number of materials and types of shield, hood, sleeve, light shroud, baffle or other physical attenuator can be used for eliminating or reducing stray light.

The systems herein can further comprise a photodiode. The systems herein can further comprise a plurality of photodiodes. The photodiode can continuously monitor and directly trip the interlock on the laser for both an underpower and overpower event. The photodiode can detect beam shape discrepancy that could indicate a diffuser failure. The photodiode can be placed at one, two, three or more locations in the laser beam path. The photodiode can be placed prior to the diffuser. The photodiodes can be placed after the diffuser to detect beam shape discrepancy that could indicate a diffuser failure. Laser classification requires a specific laser beam spot size of the diffuser. While larger beam spot sizes enable a high laser power while maintaining safe emission levels, smaller beam spot sizes reduce the obstruction required to direct the beam into the imaging pathway and provide increased sensitivity to fluorescence. Baffles to reduce reflections or stray light. Includes crescent shaped baffle on the dichroic to prevent microscope VIS light from reflecting back into VIS camera. Other baffles to reduce excitation reflections. The system shown in FIG. 4 can employ objective lenses with different f-numbers. Optimizing NIR sensitivity allows greater depth of field in the visible camera images. Further, such configurations allow for lower cost lenses with smaller optical volumes. The NIR resolution requirement can be low compared to the visible and chromatic correction from 400-1000 nm are not required. In some embodiments, the system NIR resolution is less than or equal to the VIS resolution. Such reduced resolution can enable optimal design of volume. Typically, as VIS light is more abundant than NIR or IR light, the system can be designed to maximize capture of photons of light in the NIR, IR or other range to obtain a better NIR, IR, or other signal to noise ratio, respectively. Increasing the NIR signal to noise ratio can be done in a number of ways including lowering the resolution of the NIR sensors (i.e., the use of a lower resolution sensor has larger pixel size to optimize collection of NIR photons which is more efficient (better signal to noise). Alternatively, the NIR signal to noise ratio can be increased using a faster lens (smaller F-number). Generally, NIR resolution can be less than or equal to VIS resolution in such embodiments, however if the NIR sensor is sensitive enough, smaller pixel sizes can be used and still obtain a sufficient NIR signal to noise ratio. Consequently, in some embodiments, the system NIR resolution is greater than the VIS resolution. It is recognized that focal length and F-number can further affect NIR resolution or VIS resolution in the system, and such can be adjusted and optimized accordingly. The systems herein can further comprise a baffle, a hood or both attached to the diffuser. The baffle, hood, or both can reduce stray light received by the notch filter, or LP filter on camera lens. The baffle for the VIS light from scope can have a moon shape. The baffle, hood, or both can further prevent the long tails of the top-hat diffuser profile from illuminating the filter on the camera lens at a large angle of incidence, and being transmitted through the filter, whereby the stray light could reach the imaging detector. Reducing the angle of incidence on the filter is required as steep filters cannot accommodate large variations in angle of incidence.

The systems herein can further comprise an ex-vivo docking station configured to allow use of the imaging head without the microscope. The ex-vivo docking station can comprise an optomechanical tub/tray/frame separate from enclosure, to enable safe imaging and control of visible and NIR illumination. The ex-vivo docking station enables controlled imaging for, in one example, determining reference targets. The top window, the bottom window, or both of the ex-vivo docking station can be sealed for cleanability to reduce the volume of fluids entering the imaging head.

The systems herein can further comprise a drape. The drape can be configured to surround at least a portion of the microscope head to maintain sterility therein. The drape can comprise a transparent window for viewing the sample. The drape can be compatible with current operating rooms draping systems.

In some cases, the imaging head on the microscope further comprises one or more of a flange, a rib, a guide configured to enable easy and precise attachment to the head to the microscope. In some cases, the imaging head on the microscope has a shape, a contour, or both that enable smooth integration and minimal cable interference from during attachment of the imaging head and the microscope. In some cases, the imaging head can further comprise an arrow, a symbol, a text or any combination thereof to describe or annotate proper connection of the imaging head to the microscope. The arrow, symbol, text or any combination thereof, can be adhered to or directly machined onto the imaging head. In further embodiments, the shape of the imaging head, the imaging cable, or both can be configured for efficient movement and reduced drag. Further, the imaging head can comprise a seal enhancing the sealability of the connections of the head to the scope (e.g., the top/bottom windows) aids in maintaining smooth operation and cleanliness of the device

In some embodiments, the system comprises two or more NIR indicators. In some embodiments, one NIR indicator is in the front of the device and another NIR indicator is at the bottom of the device. In some embodiments, contralateral illumination is automatically disabled when the head is inserted onto the microscope. In order to view the sample without fluorescence, a dark frame can be subtracted from any fluorescence caused by the microscope illumination. The dark frame can be applied mechanically, electronically, or by an image processing software. The systems herein can comprise a second source of illumination to prevent formations of shadow within valleys, depressions and uneven surfaces in the tissue created during surgery. However, in some cases, the second source of illumination is periodically dimmed or turned off to prevent interference with additional optical components.

In some embodiments, the systems and methods herein only include a VIS/NIR or a VIS/IR camera that is configured to sense both visible and NIR or IR signals. In some embodiments, the sensitivity for visible and NIR or IR signal is different. In some embodiments, both cameras are on a single stage. In some embodiments, both cameras are looking at the same area and focus together. In some embodiments, the field of view, aperture, focal length, depth of field, or any other parameters of both cameras are identical. In some embodiments the field of view, aperture, focal length, depth of field, or any other parameters of both cameras are not the same (e.g. aperture). In some embodiments, the systems and methods herein only include a NIR or IR camera. In some embodiments, the capture of visible frame, trigger frames (or NIR or IR frames), and dark frames can be in the same sequence. In some embodiments, there can be additional pair(s) of excitation sources and notch filters for illuminating the source with different excitation wavelengths. For example, frames 1, 2, 3, 4, and 5 (such that each frame is excited by a different wavelength—e.g., exciting different fluorophores per frame, and also one visible (white) and one dark frame)—thus the sequence of 1, 2, 3, 4, and 5 enables visualization of 3 different fluorophores simultaneously (and one white, one dark) in a single frame. With this flexibility, any number of frames and fluorophores can be imaged to allow detection of multiple fluorophores emitting at different wavelengths (e.g., on the same molecule and/or in the same sample being tested). Thus, the systems and methods herein not only apply to dyes that are NIR fluorophores, but a variety of sources that emit light (e.g., dyes which emit in green, red and infrared wavelengths). For example, various dyes that could be conjugated to peptides can be imaged with the systems and methods herein. In some embodiments, how a sample can be imaged (e.g., with or without use of a non-specific dye in normal tissue (contrast) with a different dye on targeting molecule that that homes, targets, migrates to, is retained by, accumulates in, and/or binds to, or is directed to an organ, organ substructure, tissue, target, cell or sample) can be adjusted or tested using the systems and methods herein.

Using the systems and methods herein, autofluorescence in an organ, organ substructure, tissue, target, cell or sample can be detected. Moreover, based on their autofluorescence profile, different biological structures (e.g., organ, organ substructure, tissue, target, cell or sample) can be distinguished at various wavelengths. Such autofluorescence can be enhanced and further distinguished by introducing an exogenous contrast or imaging agent, or any combination thereof. Moreover, using the systems and methods herein, fluorophores that home, target, migrate to, are retained by, accumulate in, and/or bind to, or are directed to an organ, organ substructure, tissue, target, cell or sample can be detected, whether such fluorophore is alone, conjugated, fused, linked, or otherwise attached to a chemical agent or other moiety, small molecule, therapeutic, drug, chemotherapeutic, protein, peptide, antibody protein or fragment of the foregoing, and in any combination of the foregoing. For example, human serum albumin (HSA) can be conjugated to a fluorophore and thereby increase its retention within the vasculature and its half-life. Peptides, antibodies, or antibody fragments can be engineered to target specific tissues of interest, for example vascular endothelium or nerves, so that these structures are stably labeled for the duration of a surgical or diagnostic procedure. Conjugates can be created that are non-fluorescent until they are activated in the presence of the diseased tissue or other condition to be detected. Examples include peptide moieties that are cleaved by cathepsins or matrix metalloproteinases that can be used to detect atherosclerotic plaques, tumor microenvironment, or other areas of abnormal tissue or inflammation. For example, the fluorophore is a fluorescent agent emitting electromagnetic radiation at a wavelength between 650 nm and 4000 nm, such emissions being used to detect such agent in an organ, organ substructure, tissue, target, cell or sample using the systems and methods herein. In some embodiments the fluorophore is a fluorescent agent is selected from the group consisting of non-limiting examples of fluorescent dyes that could be used as a conjugating molecule (or each class of molecules) in the present disclosure include DyLight-680, DyLight-750, VivoTag-750, DyLight-800, IRDye-800, VivoTag-680, Cy5.5, or an indocyanine green (ICG) and any derivative of the foregoing. In some embodiments, near infrared dyes often include cyanine dyes. Additional non-limiting examples of fluorescent dyes for use as a conjugating molecule in the present disclosure include acradine orange or yellow, ALEXA FLUORs and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GeiRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, methylene blue and any derivative thereof, nile dyes and any derivative thereof, OS680, OS750, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, topotecan, TSQ, umbelliferone, violanthrone, yellow fluorescent protein and YOYO-1. Other Suitable fluorescent dyes include, but are not limited to, fluorescein and fluorescein dyes (e.g., fluorescein isothiocyanineo rF ITC, naphthofluorescein, 4′, 5′-dichloro-2′,7′-dimethoxyfluorescein, 6-carboxyfluorescein or F AM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514.,., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in international patent application no. PCT/US2014/056177.

Moreover, using the systems and methods herein, fluorescent biotin conjugates that can act both as a detectable label and an affinity handle can be used to detect such agent in an organ, organ substructure, tissue, or sample using the systems and methods herein. Non limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, ALEXA FLUOR 488 biocytin, ALEXA FLUOR 546, ALEXA FLUOR 549, lucifer yellow cadaverine biotin-X, Lucifer yellow biocytin, Oregon green 488 biocytin, biotin-rhodamine and tetramethylrhodamine biocytin. In some other examples, the conjugates could include chemiluminescent compounds, colloidal metals, luminescent compounds, enzymes, radioisotopes, and paramagnetic labels. In some embodiments, the peptide-active agent fusions described herein can be attached to another molecule. For example, the peptide sequence also can be attached to another active agent (e.g., small molecule, peptide, polypeptide, polynucleotide, antibody, aptamer, cytokine, growth factor, neurotransmitter, an active fragment or modification of any of the preceding, fluorophore, radioisotope, radionuclide chelator, acyl adduct, chemical linker, or sugar, etc.). In some embodiments, the peptide can be fused with, or covalently or non-covalently linked to an active agent.

The systems and methods of the present disclosure can be used alone or in combination with a companion diagnostic, therapeutic or imaging agent (whether such diagnostic, therapeutic or imaging agent is a fluorophore alone, or conjugated, fused, linked, or otherwise attached to a chemical agent or other moiety, small molecule, therapeutic, drug, chemotherapeutic, protein, peptide, antibody protein or fragment of the foregoing, and in any combination of the foregoing; or used as a separate companion diagnostic, therapeutic or imaging agent in conjunction with the fluorophore or other detectable moiety is alone, conjugated, fused, linked, or otherwise attached to a chemical agent or other moiety, small molecule, therapeutic, drug, chemotherapeutic, peptide, antibody protein or fragment of the foregoing, and in any combination of the foregoing). Such companion diagnostics can utilize agents including chemical agents, radiolabel agents, radiosensitizing agents, fluorophores, imaging agents, diagnostic agents, protein, peptide, or small molecule such agent intended for or having diagnostic or imaging effect. Agents used for companion diagnostic agents and companion imaging agents, and therapeutic agents, can include the diagnostic, therapeutic and imaging agents described herein or other known agents. Diagnostic tests can be used to enhance the use of therapeutic products, such as those disclosed herein or other known agents. The development of therapeutic products with a corresponding diagnostic test, such as a test that uses diagnostic imaging (whether in vivo, ex vivo or in vitro) can aid in diagnosis, treatment, identify patient populations for treatment, and enhance therapeutic effect of the corresponding therapy. The systems and methods of the present disclosure can also be used to detect therapeutic products, such as those disclosed herein or other known agents, to aid in the application of a therapy and to measure it to assess the agent's safety and physiologic effect, e.g. to measure bioavailability, uptake, distribution and clearance, metabolism, pharmacokinetics, localization, blood concentration, tissue concentration, ratio, measurement of concentrations in blood and/or tissues, assessing therapeutic window, extended visibility window, range and optimization, and the like of the therapeutic agent. Such The systems and methods can be employed in the context of therapeutic, imaging and diagnostic applications of such agents. Tests also aid therapeutic product development to obtain the data FDA uses to make regulatory determinations. For example, such a test can identify appropriate subpopulations for treatment or identify populations who should not receive a particular treatment because of an increased risk of a serious side effect, making it possible to individualize, or personalize, medical therapy by identifying patients who are most likely to respond, or who are at varying degrees of risk for a particular side effect. Thus, the present disclosure, in some embodiments, includes the joint development of therapeutic products and diagnostic devices, including the systems and methods herein (used to detect the therapeutic and/or imaging agents themselves, or used to detect the companion diagnostic or imaging agent, whether such diagnostic or imaging agent is linked to the therapeutic and/or imaging agents or used as a separate companion diagnostic or imaging agent linked to the peptide for use in conjunction with the therapeutic and/or imaging agents) that are used in conjunction with safe and effective use of the therapeutic and/or imaging agents as therapeutic or imaging products. Non-limiting examples of companion devices include a surgical instrument, such as an operating microscope, confocal microscope, fluorescence scope, exoscope, endoscope, or a surgical robot and devices used in biological diagnosis or imaging or that incorporate radiology, including the imaging technologies of X-ray radiography, magnetic resonance imaging (MRI), medical ultrasonography or ultrasound, endoscopy, elastography, tactile imaging, thermography, medical photography and nuclear medicine functional imaging techniques as positron emission tomography (PET) and single-photon emission computed tomography (SPECT). Companion diagnostics and devices can comprise tests that are conducted ex vivo, including detection of signal from tissues or cells that are removed following administration of the companion diagnostic to the subject, or application of the companion diagnostic or companion imaging agent directly to tissues or cells following their removal from the subject and then detecting signal. Examples of devices used for ex vivo detection include fluorescence microscopes, flow cytometers, and the like. Moreover, the systems and methods herein for such use in companion diagnostics can be used alone or alongside, in addition to, combined with, attached to or integrated into an existing surgical microscope, confocal microscope, fluorescence scope, exoscope, endoscope, or a surgical robot, including a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., RetinaVue 100, RetinaVue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPAL1 NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz. Moreover, in some embodiments, the imaging, diagnostic, detecting and therapeutic methods herein are performed using the systems described herein alongside, in addition to, combined with, attached to, or integrated into such an existing surgical microscope, confocal microscope, fluorescence scope, exoscope, endoscope, surgical robot, microscope, exoscope, or endoscope as described above.

Any additional surgical microscope, confocal microscope, fluorescence scope, exoscope, endoscope, or surgical robot systems can be used. The surgical microscope, confocal microscope, fluorescence scope, exoscope, endoscope, or surgical robot systems can be provided by, for example, Carl Zeiss A/G, Leica Microsystems, Leica Biosystems, Haag-Streit (5-1000 or 3-1000 systems), or Intuitive Surgical (e.g.: da Vinci surgical robot system), or any other manufacturer of such systems.

The systems and methods herein can be used to detect one or more detectable agents, affinity handles, fluorophores, or dyes, two or more, three, four five and up to ten or more such detectable agents, affinity handles, fluorophores, or dyes in a given sample (e.g., organ, organ substructure, tissue, or sample).

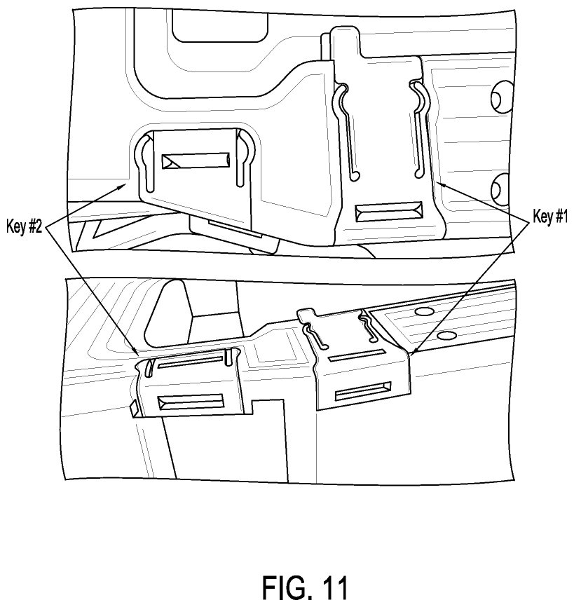

FIG. 11 shows an exemplary embodiment for the lock and key of the imaging head. The imaging head FIGS. 7A & 12 of the imaging system herein locks onto the microscope by two independent keys, where each key can be sufficient for restraint of the head to the scope. In some cases, this key mechanism does not require tools for removing of any existing hardware on the microscope, allowing quick and easy insertion or removal of the device prior or after surgical procedures.

### Image Processing

In some embodiments, the systems and methods herein allow for reinforcement and dropping off of NIR or IR frames as required based on the signal strength. In some embodiments, it can be determined how many NIR or IR frames need to be captured before performing the above-mentioned processing. If the fluorescence light from the tissue is very bright, only 2 or 3 frames instead of 4 frames are to be added for every displayed frame. Conversely, if the signal is very low, 6-9 or more frames can be captured before capturing the frame with excitation source OFF. This allows the system to reinforce or drop NIR or IR frames as required and dynamically change the sensitivity of the imaging system.

Referring to FIG. 7A, in a particular embodiment, the visible light from lamp of the surgical microscope is always on (i.e., continuous wave (CW)) while the visible camera is switched between on and off regularly. In this embodiment, the laser light is on for every 4 frames of NIR or IR frames, so that fluorescence light from such 4 frames is added for an NIR or IR image displayed, the excitation sources light is then turned off for a dark frame to provide baseline ambient light in the imaging background to be removed from the NIR or IR image.

In some embodiments, the dark frame exposure time and gain values matches the NIR or IR frame. There is flexibility in the dark frame exposure relative to the NIR or IR frame exposure. Mathematically, it can be an exact match except for the excitation source being off. In other cases, the frame can be of a different exposure and digitally matched to the NIR or IR frames. In some embodiments, the NIR frame's exposure can be a multiple of the dark frame exposure (either longer or shorter) and can be scaled to match the NIR frame exposure mathematically during image processing. In some embodiments, the exposure time for each frame can be dynamically changed.

In some embodiments, the visible camera captures the frames at a fixed frame rate and optionally after each visible image is captured, the NIR or IR frame buffer is checked, if the buffer is updated with the latest captured NIR or IR image, the image is added to the visible light image. In some embodiments, when an older NIR or IR image (as the case can be) is in the buffer, the older image is added to display, thus there can be asynchronous frame capture between visible and infrared fluorescence images. In some embodiments, this is advantageous to achieve independent of the frame rate of the fluorescence image superimposed on the visible image, which can be faster or slower, the frame rate of the output image (visible and fluorescence image) is full video rate (i.e., without time lag). In some embodiments, the video rate without time lag provided by the systems and methods herein advantageously enables the user to fine tune or simply adjust the image to maximize its visibility, clarity, operation and use in real time.

In some embodiments, the systems and methods herein use a transistor-transistor-logic (TTL) trigger signal for camera frame capture. In some embodiments, the duty cycle of the TTL trigger for camera frame capture is used to drive the excitation source's illumination. In some embodiments, one or more TTL triggers for camera frame capture is used to drive the excitation source's illumination

In some embodiments, various image processing technologies can be used on the NIR or IR images and/or visible light images, thereby facilitating display of color maps or contour images.

In some embodiments, images herein are processed by a digital processing device, a processor, or the like. In some embodiments, image processing herein includes: image reconstruction image filtering, image segmentation, addition of two or more images, subtraction of one or more images from image(s), image registration, pseudo coloring, image masking, image interpolation, or any other image handling or manipulation.

In some embodiments, images herein are displayed to a digital display and controlled by a digital processing device, a processor, or the like. In some embodiments, a digital processing device, a processor, or the like herein enable the surgeon or other users to select image type(s) to be displayed. In some embodiments, image processing is performed by an application specific integrated circuit (ASIC), located within one or more of the cameras in the imaging head, providing for the fully-processed composite image to be transmitted from the imaging head. Use of the ASIC for image processing reduces the bandwidth requirements for the cable, and the subsequent processing requirements on the ‘display side’.

In some embodiments, false or pseudo coloring is used on the NIR or IR images or visible light images. Referring to FIGS. 10A-10C, in a particular embodiment, the visible light image is colored differently, in black (FIG. 10A), white (FIG. 10B) or red (FIG. 10C), while the NIR image has false color to increase the contrast on the images over the background visible light. In these embodiments, the superimposed composite image with both fluorescent light and visible light shows the tumor tissue 106a, 106b with different signal intensity and its surrounding structures. Such difference in signal intensity is caused by different level of tissue uptake of fluorescent dye(s).

Referring to FIG. 7B, the systems and methods provide the option to view the fluorescence image superimposed on the visible image or the fluorescence image alone, or view the visible and NIR or IR images side-by-side thus providing the user flexibility with image visualization. In some embodiments, the images, visible or fluorescent images are two-dimensional image frames that can be stacked to make three-dimensional volumetric image(s).

In some embodiments, the tumor is automatically, semi-automatically, or manually contoured in visible light and/or NIR or IR image during image processing so that the tumor and the tumor boundary can be better visualized by a surgeon or any other medical professional. In some embodiments, the NIR or IR image is integrated along x axis and/or y axis so that a one dimensional fluorescence signal profile is generated.

### Computing System

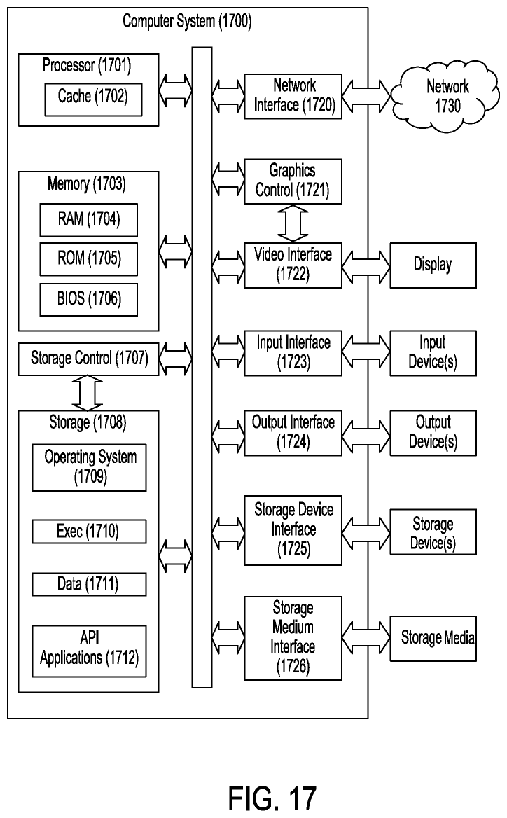

Referring to FIG. 17, a block diagram is shown depicting an exemplary machine that includes a computer system 1700 (e.g., a processing or computing system) within which a set of instructions can execute for causing a device to perform or execute any one or more of the aspects and/or methodologies for static code scheduling of the present disclosure. The components in FIG. 17 are examples only and do not limit the scope of use or functionality of any hardware, software, embedded logic component, or a combination of two or more such components implementing particular embodiments.

Computer system 1700 can include one or more processors 1701, a memory 1703, and a storage 1708 that communicate with each other, and with other components, via a bus 1740. The bus 1740 can also link a display 1732, one or more input devices 1733 (which can, for example, include a keypad, a keyboard, a mouse, a stylus, etc.), one or more output devices 1734, one or more storage devices 1735, and various tangible storage media 1736. All of these elements can interface directly or via one or more interfaces or adaptors to the bus 1740. For instance, the various tangible storage media 1736 can interface with the bus 1740 via storage medium interface 1726. Computer system 1700 can have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1700 includes one or more processor(s) 1701 (e.g., central processing units (CPUs) or general purpose graphics processing units (GPGPUs)) that carry out functions. Processor(s) 1701 optionally contains a cache memory unit 1702 for temporary local storage of instructions, data, or computer addresses. Processor(s) 1701 are configured to assist in execution of computer readable instructions. Computer system 1700 can provide functionality for the components depicted in FIG. 17 as a result of the processor(s) 1701 executing non-transitory, processor-executable instructions embodied in one or more tangible computer-readable storage media, such as memory 1703, storage 1708, storage devices 1735, and/or storage medium 1736. The computer-readable media can store software that implements particular embodiments, and processor(s) 1701 can execute the software. Memory 1703 can read the software from one or more other computer-readable media (such as mass storage device(s) 1735, 1736) or from one or more other sources through a suitable interface, such as network interface 1720. The software can cause processor(s) 1701 to carry out one or more processes or one or more steps of one or more processes described or illustrated herein. Carrying out such processes or steps can include defining data structures stored in memory 1703 and modifying the data structures as directed by the software.

The memory 1703 can include various components (e.g., machine readable media) including, but not limited to, a random access memory component (e.g., RAM 1704) (e.g., static RAM (SRAM), dynamic RAM (DRAM), ferroelectric random access memory (FRAM), phase-change random access memory (PRAM), etc.), a read-only memory component (e.g., ROM 1705), and any combinations thereof. ROM 1705 can act to communicate data and instructions unidirectionally to processor(s) 1701, and RAM 1704 can act to communicate data and instructions bidirectionally with processor(s) 1701. ROM 1705 and RAM 1704 can include any suitable tangible computer-readable media described below. In one example, a basic input/output system 1706 (BIOS), including basic routines that help to transfer information between elements within computer system 1700, such as during start-up, can be stored in the memory 1703.

Fixed storage 1708 is connected bidirectionally to processor(s) 1701, optionally through storage control unit 1707. Fixed storage 1708 provides additional data storage capacity and can also include any suitable tangible computer-readable media described herein. Storage 1708 can be used to store operating system 1709, executable(s) 1710, data 1711, applications 1712 (application programs), and the like. Storage 1708 can also include an optical disk drive, a solid-state memory device (e.g., flash-based systems), or a combination of any of the above. Information in storage 1708 can, in appropriate cases, be incorporated as virtual memory in memory 1703.

In one example, storage device(s) 1735 can be removably interfaced with computer system 1700 (e.g., via an external port connector (not shown)) via a storage device interface 1725. Particularly, storage device(s) 1735 and an associated machine-readable medium can provide non-volatile and/or volatile storage of machine-readable instructions, data structures, program modules, and/or other data for the computer system 1700. In one example, software can reside, completely or partially, within a machine-readable medium on storage device(s) 1735. In another example, software can reside, completely or partially, within processor(s) 1701.

Bus 1740 connects a wide variety of subsystems. Herein, reference to a bus can encompass one or more digital signal lines serving a common function, where appropriate. Bus 1740 can be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1700 can also include an input device 1733. In one example, a user of computer system 1700 can enter commands and/or other information into computer system 1700 via input device(s) 1733. Examples of an input device(s) 1733 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1733 can be interfaced to bus 1740 via any of a variety of input interfaces 1723 (e.g., input interface 1723) including, but not limited to, serial, parallel, game port, USB, FIREWIRE, THUNDERBOLT, or any combination of the above.

In particular embodiments, when computer system 1700 is connected to network 1730, computer system 1700 can communicate with other devices, specifically mobile devices and enterprise systems, distributed computing systems, cloud storage systems, cloud computing systems, and the like, connected to network 1730. Communications to and from computer system 1700 can be sent through network interface 1720. For example, network interface 1720 can receive incoming communications (such as requests or responses from other devices) in the form of one or more packets (such as Internet Protocol (IP) packets) from network 1730, and computer system 1700 can store the incoming communications in memory 1703 for processing.

Computer system 1700 can similarly store outgoing communications (such as requests or responses to other devices) in the form of one or more packets in memory 1703 and communicated to network 1730 from network interface 1720. Processor(s) 1701 can access these communication packets stored in memory 1703 for processing.

Examples of the network interface 1720 include, but are not limited to, a network interface card, a modem, and any combination thereof. Examples of a network 1730 or network segment 1730 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1730, can employ a wired and/or a wireless mode of communication. In general, any network topology can be used.

Information and data can be displayed through a display 1732. Examples of a display 1732 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1732 can interface to the processor(s) 1701, memory 1703, and fixed storage 1708, as well as other devices, such as input device(s) 1733, via the bus 1740. The display 1732 is linked to the bus 1740 via a video interface 1722, and transport of data between the display 1732 and the bus 1740 can be controlled via the graphics control 1721. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1732, computer system 1700 can include one or more other peripheral output devices 1734 including, but not limited to, an audio speaker, a printer, a storage device, and any combinations thereof. Such peripheral output devices can be connected to the bus 1740 via an output interface 1724. Examples of an output interface 1724 include, but are not limited to, a serial port, a parallel connection, a USB port, a FIREWIRE port, a THUNDERBOLT port, and any combinations thereof.

In addition or as an alternative, computer system 1700 can provide functionality as a result of logic hardwired or otherwise embodied in a circuit, which can operate in place of or together with software to execute one or more processes or one or more steps of one or more processes described or illustrated herein. Reference to software in this disclosure can encompass logic, and reference to logic can encompass software. Moreover, reference to a computer-readable medium can encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein can be embodied directly in hardware, in a software module executed by one or more processor(s), or in a combination of the two. A software module can reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360@, Microsoft Xbox One, Nintendo® Wii®, Nintendo® Wii U®, and Ouya®.

### Digital Processing Device

In some embodiments, the systems and methods described herein include a digital processing device, a processor, or use of the same. In further embodiments, the digital processing device includes one or more hardware central processing units (CPUs) and/or general-purpose graphics processing units (GPGPUs), or special purpose GPGCUs that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. In addition, in accordance with the description herein, devices include also partitioning the signal processing and computation between a unit located proximally to the imaging optics (e.g. a FPGA or DSP), and a ‘back end’ PC. It is understood that distribution of the processing can be performed between various locations.

In some embodiments, the digital processing device includes an operating system configured to perform executable instructions. The operating system is, for example, software, including programs and data, which manages the device's hardware and provides services for execution of applications.

In some embodiments, the device includes a storage and/or memory device. The storage and/or memory device is one or more physical apparatuses used to store data or programs on a temporary or permanent basis.

In some embodiments, the digital processing device includes a display to send visual information to a user.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

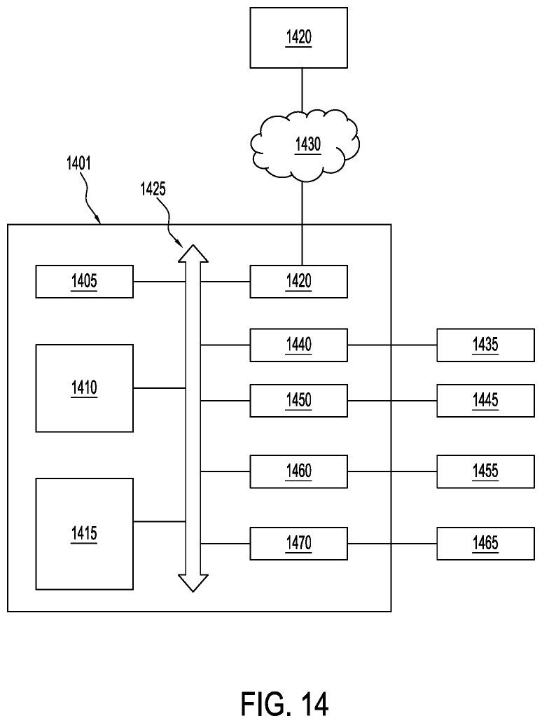

Referring to FIG. 14, in a particular embodiment, an exemplary digital processing device 1401 is programmed or otherwise configured to control imaging and image processing aspects of the systems herein. In this embodiment, the digital processing device 1401 includes a central processing unit (CPU, also “processor” and “computer processor” herein) 1405, which can be a single core or multi core processor, or a plurality of processors for parallel processing. The digital processing device 1401 also includes memory or memory location 1410 (e.g., random-access memory, read-only memory, flash memory), electronic storage unit 1415 (e.g., hard disk), communication interface 1420 (e.g., network adapter, network interface) for communicating with one or more other systems, and peripheral devices, such as cache, other memory, data storage and/or electronic display adapters. The peripheral devices can include storage device(s) or storage medium 1465 which communicate with the rest of the device via a storage interface 1470. The memory 1410, storage unit 1415, interface 1420 and peripheral devices are in communication with the CPU 1405 through a communication bus 1425, such as a motherboard. The storage unit 1415 can be a data storage unit (or data repository) for storing data. The digital processing device 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some embodiments is a telecommunication and/or data network. The network 1430 can include one or more computer servers, which can enable distributed computing, such as cloud computing. The network 1430, in some embodiments with the aid of the device 1401, can implement a peer-to-peer network, which can enable devices coupled to the device 1401 to behave as a client or a server.

Continuing to refer to FIG. 14, the digital processing device 1401 includes input device(s) 1445 to receive information from a user, the input device(s) in communication with other elements of the device via an input interface 1450. The digital processing device 1401 can include output device(s) 1455 that communicates to other elements of the device via an output interface 1460.

Continuing to refer to FIG. 14, the memory 1410 can include various components (e.g., machine readable media) including, but not limited to, a random-access memory component (e.g., RAM) (e.g., a static RAM “SRAM”, a dynamic RAM “DRAM, etc.), or a read-only component (e.g., ROM). The memory 1410 can also include a basic input/output system (BIOS), including basic routines that help to transfer information betweF5-10en elements within the digital processing device, such as during device start-up, can be stored in the memory 1410.

Continuing to refer to FIG. 14, the CPU 1405 can execute a sequence of machine-readable instructions, which can be embodied in a program or software. The instructions can be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and write back. The CPU 1405 can be part of a circuit, such as an integrated circuit. One or more other components of the device 1401 can be included in the circuit. In some embodiments, the circuit is an application specific integrated circuit (ASIC) or a field programmable gate array (FPGA).

Continuing to refer to FIG. 14, the storage unit 1415 can store files, such as drivers, libraries and saved programs. The storage unit 1415 can store user data, e.g., user preferences and user programs. The digital processing device 1401 in some embodiments can include one or more additional data storage units that are external, such as located on a remote server that is in communication through an intranet or the Internet. The storage unit 1415 can also be used to store operating system, application programs, and the like. Optionally, storage unit 1415 can be removably interfaced with the digital processing device (e.g., via an external port connector (not shown)) and/or via a storage unit interface. Software can reside, completely or partially, within a computer-readable storage medium within or outside of the storage unit 1415. In another example, software can reside, completely or partially, within processor(s) 1405.

Continuing to refer to FIG. 14, the digital processing device 1401 can communicate with one or more remote computer systems 1402 through the network 1430. For instance, the device 1401 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants. In some embodiments, the remote computer system is configured for image and signal processing of images acquired using the image systems herein. In some embodiments, the imaging systems herein allows partitioning of image and signal processing between a processor in the imaging head (e.g. based on a MCU, DSP or FPGA) and a remote computer system, i.e., a back-end server.

Continuing to refer to FIG. 14, information and data can be displayed to a user through a display 1435. The display is connected to the bus 1425 via an interface 1440, and transport of data between the display other elements of the device 1401 can be controlled via the interface 1440.

Methods as described herein can be implemented by way of machine (e.g., computer processor) executable code stored on an electronic storage location of the digital processing device 1401, such as, for example, on the memory 1410 or electronic storage unit 1415. The machine executable or machine-readable code can be provided in the form of software. During use, the code can be executed by the processor 1405. In some embodiments, the code can be retrieved from the storage unit 1415 and stored on the memory 1410 for ready access by the processor 1405. In some situations, the electronic storage unit 1415 can be precluded, and machine-executable instructions are stored on memory 1410.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some embodiments, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions can be implemented as program modules, such as functions, objects, Application Programming Interfaces (APIs), data structures, and the like, that perform particular tasks or implement particular abstract data types. In light of the disclosure provided herein, those of skill in the art will recognize that a computer program can be written in various versions of various languages.

The functionality of the computer readable instructions can be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Indications and Methods

The systems and methods herein can be used to identify presence of health or disease, in diagnosis, imaging, health monitoring and the like in a given sample (e.g., organ, organ substructure, tissue, or sample, whether ex-vivo or in situ).

For example, fluorescence angiography is useful during certain neurosurgical procedures in the brain and spinal cord. Repair of blood vessel defects such as a vascular lesion, vascular malformation, vascular abnormality, aneurysm, arteriovenous malformation, cavernous malformation, venous malformation, lymphatic malformation, capillary telangiectasia, mixed vascular malformation, or spinal dural arteriovenous fistula, and the like, requires imaging of the defect architecture, confirmation that the defect is successfully isolated prior to repair, and confirmation that repaired vessels have restored proper blood flow and patency. Vessel patency is particularly crucial in the CNS to avoid neurologic damage or death that can result from undetected bleeding into these tissues. Neurosurgical microscopes, neuroendoscopes, endovascular endoscopes, and robotic surgical systems including the systems and methods described herein may all be used in this setting. Removal of CNS tumors such as pituitary adenoma is another setting in which fluorescence angiography can be applied to improve safety and efficacy of treatment. The visualization of vascular flow to the tumor and verification that the tumor has been removed without residual bleeding are both important uses for this technology.

Fluorescence angiography, cholangiography, lymphography, and the like are useful in support of a variety of surgical interventions. The systems and methods described herein can be used in various cardiovascular surgeries, including aneurysm repair, valve replacement, arteriovenous malformation, cavernous malformation or a venous malformation, a lymphatic malformation, a capillary telangiectasia, a mixed vascular malformation, or a spinal dural arteriovenous fistula, repair or bypass, arterial bypass, and the like for visualization of blood flow and vessel patency. The systems and methods described herein can be used in plastic surgery, trauma surgery, reconstructive surgery, and the like for vascular mapping and for assessment of tissue perfusion. Tissue perfusion is of particular importance in flap reconstructions and in anastomoses of the gastrointestinal tract, for example following colorectal cancer surgery or esophagectomy, as tissue ischemia following such surgeries can result in loss of tissue and graft failure or leaking anastomosis. Fluorescence lymphography using systems and methods described herein is useful for demonstrating flow of the lymphatic vessels, for example to support re-routing of lymphatic drainage to treat lymphedema.

The systems and methods described herein are useful for visualization of organs or organ segments in a variety of surgical procedures. Liver segments can be imaged following intra-arterial dye injection during partial hepatectomy. Perfusion and bile production can be assessed following partial or total liver transplantation. Other hepatobiliary surgeries, including resection of liver cancers or metastases, are also supported by angiography or cholangiography. Contrast between the kidney and adrenal gland can be achieved using fluorescent dyes or conjugates that are cleared through renal filtration. This procedure can help in differentiating the adrenal gland from the kidney, for example to avoid kidney damage during removal of the adrenal glands. The ureters can also be identified using these methods to avoid damage to them during uro-abdominal surgeries. Abnormally vascularized tissues, such as endometriosis or tumors, can be identified and removed using these methods.

Coupled with a targeting moiety that binds specifically to nerves, fluorescence imaging systems and methods described herein can be used to visualize nerves during surgery to avoid damage. This is important during surgeries in highly innervated areas, particularly where damage to the nerves can result in significant morbidity. Examples include facial nerves, visceral nerves, and cavernous nerves. For example, cavernous nerves are important for penile and clitoral erection, and thus important for erectile function. Moreover, vascular injury and diseases affect blood vessels and blood flow. For example, affected cavernous nerves or decreased blood flow to the penis can cause erectile dysfunction. Moreover, the cavernous nerves of the penis are frequently damaged during prostate cancer surgery. Vascular reconstructive surgery is one way to improve blood flow to the penis to help a man with erectile dysfunction (ED).

Coupled with a targeting moiety that binds or accumulates specifically in abnormal vascular tissue, fluorescence imaging systems and methods described herein can be used to identify vascular abnormalities during surgery. For example, a cavernoma is a cluster of abnormal blood vessels, usually found in the brain and spinal cord. They are sometimes known as cavernous angiomas, cavernous hemangiomas, or cerebral cavernous malformation (CCM). A typical cavernoma looks like a raspberry. Cavernomas are treated by microsurgical resection or stereotactic radiosurgery if the patient is experiencing severe symptoms, such as intractable seizures, progressive neurological deterioration, one severe hemorrhage in a noneloquent region of the brain, or at least two severe hemorrhages in eloquent brain. The systems and methods herein can be used to detect, image and treat cavernous malformation, cavernous angiomas, cavernous hemangiomas, or cerebral cavernous malformation (CCM) including via surgery.

Similarly, the systems and methods herein can be used to detect, image and treat arteriovenous malformation including via surgery. An arteriovenous malformation (AVM) is an abnormal tangle of blood vessels connecting arteries and veins, which disrupts normal blood flow and oxygen circulation. Arteries are responsible for taking oxygen-rich blood from the heart to the brain. Veins carry the oxygen-depleted blood back to the lungs and heart. AVM treatment sometimes requires a combination of treatments, including surgery, embolization and radiation.

Disruptions of the ophthalmic vasculature occur as a result of diseases such as diabetes, glaucoma, or Susac's syndrome, secondary to trauma, or spontaneously. The systems and methods described herein are useful in diagnosis and in treatment targeting and/or monitoring of such disruptions. These can include macular edema, macular ischemia, age-related macular degeneration, retinal tear, retinal degeneration, retinal artery occlusion, retinal vein occlusion, and the like. Treatment of tumors in the eye often require intraocular injection of chemotherapeutics, which requires careful monitoring to ensure accurate delivery to the tumor while minimizing damage to normal structures within or surrounding the eye. In some instances, endogenous fluorescence of the chemotherapeutic, such as topotecan, can be monitored. In other instances, a tracer dye can be administered with the chemotherapeutic to facilitate imaging.

Certain types of cancer, such as head and neck cancer or sarcomas of the extremities, may be treated using superselective intra-arterial chemotherapy. This method can improve prognosis and spare normal organ function but requires precise identification of the vessels supplying blood to the cancer tissue. The systems and methods described herein are useful for fluorescence imaging and identification of appropriate arteries prior to administration of chemotherapy.

Such systems can be useful for endovascular imaging for diagnosis and treatment monitoring in cardiovascular diseases such as atherosclerosis. Examination of features such as lumen dimensions, plaque burden, remodeling, lipid components, cap thickness, neo-angiogenesis, and inflammation are used to diagnose plaque instability; fluorescence imaging in combination with other technologies can improve these assessments. Following stent placement, fluorescence angiography can be used to detect vessel restenosis.

The systems and methods described herein are useful in non-invasive diagnosis and monitoring of tissue perfusion, for example in chronic wounds or limb/extremity ischemia.

The systems and methods described herein are useful in microvasculature imaging. For example, oxyhemoglobin and deoxyhemoglobin have sequential two-color, two-photon absorption properties that can serve as endogenous contrasts in microvasculature imaging. Using a sensitive modulation transfer technique, the systems and methods described herein can image hemoglobin in red blood cells with micrometer resolution, with or without labeling using a fluorophore or other detectable compound.

The systems and methods described herein can use multispectral images to identify subcutaneous vasculature, with improved contrast in the near infrared spectrum, including detection and methods involving infrared and near-infrared imaging of superficial blood vessels.

The systems and methods described herein can be used in angiography and coronary catheterization. For example, a coronary angiogram is a procedure that uses imaging to see the heart's blood vessels. The test is generally done to visualize any restrictions in blood flow going to the heart. Coronary angiograms are part of a general group of procedures known as heart (cardiac) catheterizations. Cardiac catheterization procedures can both diagnose and treat heart and blood vessel conditions. A coronary angiogram, which can help diagnose heart conditions, is the most common type of cardiac catheterization procedure. Similarly, such systems and methods described herein can be applied to other vasculature including lymph, cerebral vasculature, organ vasculature, arteries, capillaries, veins, and the like.

The systems and methods described herein can be used in imaging and detecting cancers, e.g., for detecting and imaging angiogenesis, (i.e., the formation of new blood vessels) associated with tumors.

The systems and methods described herein can be used to diagnose, image, and detect blood vessel derived tumors and aid in their treatment through surgery and improve the health of patients through monitoring. Vascular tumors may be benign or malignant. Benign tumors form recognizable vascular channels filled with blood or lymphatic fluid. Malignant tumors are usually more solid and cellular without well-formed vascular channels. Similarly, such systems and methods described herein can be applied to other vasculature including lymph, cerebral vasculature, organ vasculature, arteries, capillaries, veins, and the like. Exemplary vessel derived tumors include those of endothelial cells, including hemangiomas, lymphangiomas, angiosarcomas, or cells supporting or surrounding blood vessels including glomus tumors, or hemangiopericytomas.

The systems and methods described herein can be used to diagnose, image, monitor and determine the outcome of heart surgery, including heart valve surgery, and treatment through surgery and improve the health of patients through monitoring.

In some applications the systems and methods disclosed herein can be used to diagnose, image, and monitor intrinsic fluorescence or autofluorescence in tissues with or without the administration of a fluorescent dye or other fluorescent agent as a contrast agent or an imaging agentpe rs e. Intrinsicpr otein fluorescence, predominantlyde rived fromt ryptophan (λ_{EX}˜280 nm, λ_{EM} —350 nm), as well as other aromatic amino acids tyrosine and phenylalanine, in proteins can be used with the systems and methods herein, for example in label-free Forster resonance energy transfer (FRET) techniques. For example, in terms of wavelength and intensity, tryptophan fluorescence is strongly influenced by its (or the protein's) local environment, which, in addition to fluorescence quenching, has been applied to study protein conformational changes. Intrinsic FRET utilizes the intrinsic fluorescence of tryptophan in conjunction with target-specific fluorescent probes as FRET donors and acceptors, respectively, for real time detection of native proteins. For example, fluorescence intensity profiles measured along the optical axis of human eye lenses can correlate with age-related nuclear cataract showing increasing concentration of fluorescent post-translational modification (PTM) towards the lens center in accord with the increased optical density in the lens nucleolus. The imaging systems and methods herein can provide spatiotemporal information of PTMs with little perturbation to the cellular environment. Significant differences between fluorescence lifetimes of “free” Trp derivatives hydroxytryptophan (OH-Trp), N-formylkynurenine (NFK), kynurenine (Kyn), hydroxykynurenine (OH-Kyn) and their residues can be measured and used to image, monitor, and diagnose disease in the eye. In addition, fundus autofluorescence (FAF) is a non-invasive retinal imaging modality used in clinical practice to provide a density map of lipofuscin, the predominant ocular fluorophore, in the retinal pigment epithelium. The imaging systems and methods herein can be used to evaluate, image, diagnose, and monitor various retinal diseases, including age related macular degeneration, macular dystrophies, retinitis pigmentosa, white dot syndromes, retinal drug toxicities, and various other retinal disorders. Moreover, autofluorescence depends on endogenous fluorophores in the tissue, which undergo a change associated with malignant transformation. This change (malignancy) can be detected as an alteration in the spectral profile and intensity of autofluorescence. Consequently, autofluorescence of tumors can be detected using the systems and methods described herein, making the systems and methods herein useful for imaging, diagnosing, and monitoring a variety of cancers. For example, bladder cancer is an exemplary cancer that autofluoresces. Fluorescence excitation wavelengths varying from 220 to 500 nm were used to induce tissue autofluorescence, and emission spectra can be measured in the 280-700 nm range. These spectra are combined to construct 2-dimensional fluorescence excitation-emission matrices (EEMs). Significant changes in fluorescence intensity of EEMs observed between normal and tumor bladder tissues are indicative of disease, the most marked differences being at the excitation wavelengths of 280 and 330 nm. Addition of contrast, fluorescent imaging agents, or target-specific fluorescent agents, can be used to further exemplify the application of the systems and methods in the detection, imaging, diagnosis, and monitoring of intrinsic tissue autofluorescence and tissue autofluorescence various applications.

Table 2 shows information of exemplary embodiments of indications and applicable organ vasculature for use with the systems and methods herein.

TABLE 2
Use of Systems and Methods in Vascular Intervention

| Indication | Intervention type | Organ System |
|---|---|---|
| Arteriovenous malformation | Neurosurgery | CNS |
| Cavernous malformation | Neurosurgery | CNS |
| Intracranial aneurysm | Neurosurgery | CNS |
| Pituitary adenoma | Neurosurgery | CNS |
| Spinal dural arteriovenous | Surgery | CNS |
| fistula | | |
| Adrenal surgery | Surgery | Endocrine |
| Thyroid surgery (parathyroid | Surgery | Endocrine |
| preservation) | | |
| Critical limb ischemia | Diagnostic | Extremities |
| Retinoblastoma | Chemotherapy | Eye |
| Diabetic macular edema | Diagnostic | Eye |
| Diabetic macular ischemia | Diagnostic | Eye |
| Diabetic retinopathy | Diagnostic | Eye |
| Macular degeneration | Diagnostic | Eye |
| Retinal artery occlusion | Diagnostic | Eye |
| Retinal vein occlusion | Diagnostic | Eye |
| Susac's syndrome | Diagnostic | Eye |
| Glaucoma | Diagnostic | Eye |
| Retinal surgery | Surgery | Eye |
| Kidney transplant | Surgery | Genitourinary |
| Ureter visualization | Surgery | Genitourinary |
| (any uro-abdominal surgery) | | |
| Kidney stones | Surgery | Genitourinary |
| Colorectal surgery | Surgery | GI |
| Esophageal anastomosis | Surgery | GI |
| Craniomaxillofacial trauma | Surgery | Head and neck |
| Liver cancer | Surgery | Hepatobiliary |
| Partial hepatectomy | Surgery | Hepatobiliary |
| Partial liver transplantation | Surgery | Hepatobiliary |
| Hepatobiliary surgery | Surgery | Hepatobiliary |
| Chronic wounds | Diagnostic | Soft tissue |
| Plastic surgery | Surgery | Soft tissue |
| Reconstructive surgery | Surgery | Soft tissue |
| Intra-arterial chemotherapy | Chemotherapy | Tumor |
| Lymphedema | Diagnostic | Vasculature |
| Atherosclerosis | Diagnostic | Vasculature |
| Endometriosis | Surgery | Viscera |

### Terms and Definitions

For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein.

As used herein A and/or B encompasses one or more of A or B, and combinations thereof such as A and B. It will be understood that although the terms “first,” “second,” “third” etc. can be used herein to describe various elements, components, regions and/or sections, these elements, components, regions and/or sections should not be limited by these terms. These terms are merely used to distinguish one element, component, region or section from another element, component, region or section. Thus, a first element, component, region or section discussed below could be termed a second element, component, region or section without departing from the teachings of the present disclosure.

The terminology used herein is for the purpose of describing particular embodiments only and is not intended to limit the present disclosure. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

As used in this specification and the claims, unless otherwise stated, the term “about,” and “approximately,” or “substantially” refers to variations of less than or equal to +/−0.1%, +/−1%, +/−2%, +/−3%, +/−4%, +/−5%, +/−6%, +/−7%, +/−8%, +/−9%, +/−10%, +/−11%, +/−12%, +/−14%, +/−15%, or +/−20% of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

As used herein, “LP” refers to longpass filters. LP filters transmit wavelengths longer than the transition wavelength and reflect a range of wavelengths shorter than the transition wavelength, as will be understood by one of ordinary skill in the art.

As used herein “SP” refers to shortpass filters. SP filters transmit wavelengths shorter than the transition wavelength and reflect a range of wavelengths longer than the transition wavelength, as will be understood by one of ordinary skill in the art.

As used herein “infrared” means any light in the infrared spectrum including light wavelengths in the IR-A (about 800-1400 nm), IR-B (about 1400 nm-3 μm) and IR-C (about 3 μm-1 mm) ranges, and near infrared (NIR) spectrums from 700 nm to 3000 nm.

As used herein, “coaxial” means that two or more light beam paths substantially overlap or are substantially parallel to each other within appropriate tolerances. That is, the axis along which a cone of light used for excitation extends along the imaging axis.

As used herein, “hot mirror”, “shortpass dichroic filter”, and “shortpass dichroic mirror” have the meaning as would be understood by one of ordinary skill in the art.

As used herein, “cold mirror”, “long pass dielectric filter”, and “longpass dichroic mirror” as used herein have the same meaning as would be understood by one of ordinary skill in the art.

As used herein, “dielectric filter”, and “dielectric mirror” as used herein can refer to a same physical element. A “dielectric filter” can refer to a device for selective transmitting. A “dielectric filter” can refer to a device for selective reflecting.

As used herein, “filter”, and “mirror” as used herein can refer to a same physical element.

Unless otherwise defined, all technical terms used herein have the same meaning as commonly understood by one of ordinary skill in the art to which this invention belongs.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein can be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

Although certain embodiments and examples are provided in the description, the inventive subject matter extends beyond the specifically disclosed embodiments to other alternative embodiments and/or uses, and to modifications and equivalents thereof. Thus, the scope of the claims appended hereto is not limited by any of the particular embodiments described herein. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures, systems, and/or devices described herein can be embodied as integrated components or as separate components.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure.

### EXAMPLES

The following illustrative examples are representative of embodiments of the software applications, systems, and methods described herein and are not meant to be limiting in any way.

### Example 1

### Use of System During Pediatric Brain Tumor Resection

This example describes use of the imaging system and/or method disclosed herein for coaxial illumination and visualization of tozuleristide fluorescence during surgical resection of a pediatric brain tumor. The imaging system of the present invention was used to image brain tissue to detect a cancer using fluorescence imaging. Surgery was performed to remove cancer from the subject.

The structure of tozuleristide is shown below:

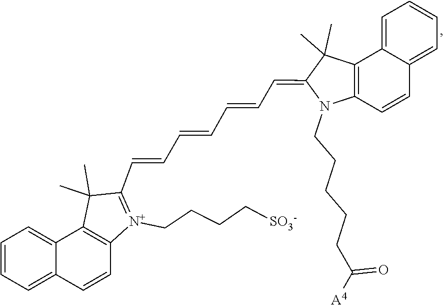

wherein A⁴ is a peptide of MCMPCFTTDHQMARRCDDCCGGRGRGKCYGPQCLCR (SEQ ID NO: 1).

Subject T613 was diagnosed with a Grade 4 Atypical Teratoid Rhabdoid Tumor (ATRT) in the posterior fossa/brain stem. Tozuleristide which is a peptide-fluorophore detectable agent (15 mg/m2 dose), was given by intravenous (IV) bolus injection about 13.5 hours prior to the start of surgery. The imaging head was attached to the Zeiss Pentero surgical microscope along with two eyepieces prior the start of surgery.

After the tumor was exposed, the imaging system was initialized and used continuously. The imaging system enabled the surgeon to view fluorescence and visible imaging together and simultaneously with the operating microscope. The surgeon noted that the imaging system was unobtrusive and easy to use, and its use did not burden or hinder surgical routine practice. Moreover, there was no need to reposition the operating microscope to view the fluorescence and visible images thus providing imaging of the surgical area together with the fluorescence imaging system during the operation, which decreased disruption to the surgical workflow.

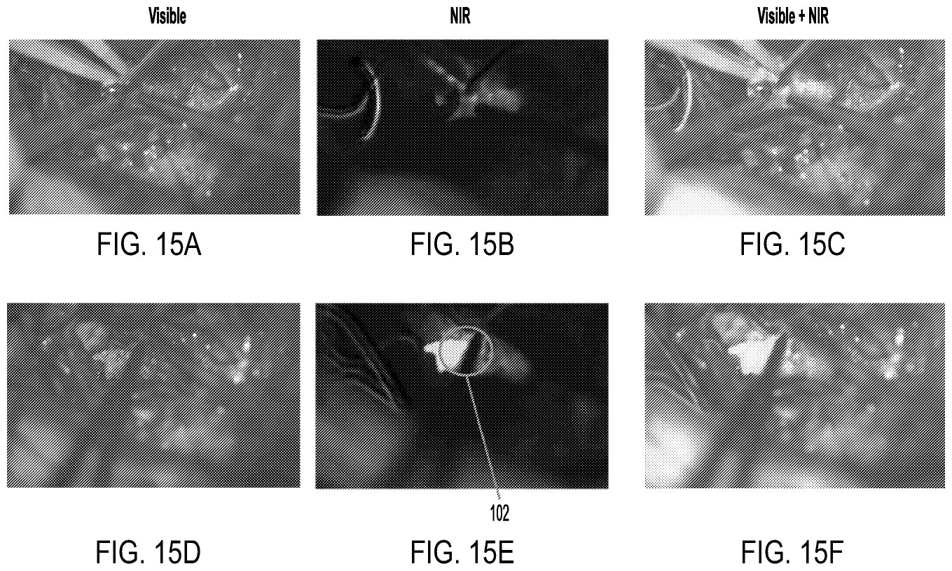

Video was captured for the duration of the tumor resection, and still images were captured of the exposed tumor. Tozuleristide fluorescence was observed in situ in the exposed tumor. FIGS. 15A-15F show images taken from the tumor resection with the near-infrared (NIR) fluorescence images of the tumor using the imaging system (FIGS. 15B and 15E) and the overlay image with the NIR fluorescence overlaid with the white light or visible light spectrum illumination (FIGS. 15C and 15F). The tumor appeared to the surgeon as a bright blue-green mass 102 in the NIR fluorescence image and in the overlay image (shown as a bright white mass in grey-scale), while the normal brain tissue appeared darker than the tumor mass in the NIR fluorescence image indicating no discernable background fluorescence in non-tumor or normal brain tissue. In the overlay image, the normal brain tissue appeared red, as it does under normal visible light or white light as shown the visible light images of the tumor (FIGS. 15A and 15D). The surgeon noted that only tumor tissue appeared fluorescent. The surgeon also noted that under normal visible light it was “somewhat difficult to distinguish tumor from normal tissue,” but with NIR fluorescence using the imaging system there was “very good distinction between tumor and normal tissue fluorescence.” The fluorescent tissue samples were demonstrated and confirmed to be viable tumor by histopathology.

This case demonstrated that the imaging system could be used continuously in an intraoperative setting to capture images and video of white light and NIR fluorescence, without disrupting the normal surgical flow. The data further demonstrated that the coaxial illumination and imaging system enabled the surgeon to visualize and precisely localize fluorescence in tumor tissues during surgery and use this information to remove tumor tissue during resection.

### Example 2

### Use of System for Angiography in Repair of CNS Vascular Defects

This example describes using the imaging systems and methods herein for the imaging, detection, monitoring, diagnosis or treatment of vascular defects (e.g., arteriovenous malformation, cavernous malformation, intracranial aneurysm) in a subject, comprising any contrast or imaging agents including an indocyanine green (ICG) or fluorescein alone or in conjunction with a peptide or active agent. The agent is administered to a subject. The subject is a human or an animal and has a vascular defect. Administration is intravenous, subcutaneous, intranasal, oral, intraperitoneal, intramuscular, or intradermal. Upon administration, the agent is targeted to vascular tissues and cells thereof or is selectively retained within the blood. The agent is then visualized using the imaging system, in conjunction with a neurosurgical operating microscope, a neuroendoscope, a vascular endoscope, or as an open imaging system. The selection of the appropriate imaging system is made by the surgeon and is dependent on the size and location of the vascular defect as well as surgical approach. The surgeon exposes the defect, and the imaging system is initialized and used continuously. The imaging system enables the surgeon to view fluorescence and visible imaging together and simultaneously with the operating microscope or other imaging system. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 3

### Use of System for Angiography in Blood or Lymph and Arteriography

This example describes use of the imaging system and/or method disclosed herein for coaxial illumination and visualization of blood or lymph in a subject. The imaging system of the present invention is used to image vascular or lymph vessels to image, monitor, diagnose, or guide treatment of disease. Surgery is performed to remove or bypass occlusions, repair vascular defects, provide for lymphatic drainage into the circulatory system to treat lymphedema, or to remove cancer or other abnormal tissue, such as endometriosis, from the subject. A contrast or imaging agent, including an indocyanine green (ICG) or fluorescein alone or in conjunction with a peptide or active agent, is administered to a subject. The subject is a human or an animal and has an occlusion that necessitates removal or bypass, or a tumor or other abnormal tissue that requires removal. Administration is intravenous, subcutaneous, intranasal, oral, intraperitoneal, intramuscular, intradermal, or by intratumoral injection. Upon administration, the agent is targeted to vascular tissues and cells thereof, lymphatic tissues and cells thereof, tumor or other abnormal vasculature, or is selectively retained within the blood or lymph. The agent is then visualized using the imaging system, in conjunction with a neurosurgical operating microscope, a neuroendoscope, a vascular endoscope, an endoscope, thoracoscope, telescope, robotic surgical system, other surgical microscope, or as an open imaging system. The selection of the appropriate imaging system is made by the surgeon and is dependent on the surgical approach. The surgeon exposes the occlusion, cancer, or other tissue, and the imaging system is initialized and used continuously. The imaging system enables the surgeon to view fluorescence and visible imaging together and simultaneously with the operating microscope or other imaging system. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 4

### Use of System for Angiography in the Eye

This example describes using the imaging systems and methods herein for the imaging, detection, monitoring, diagnosis or treatment of disease, injury, or malformation of ocular structures (e.g., diabetic macular edema, diabetic macular ischemia, diabetic retinopathy, macular degeneration, retinal artery occlusion, retinal vein occlusion, Susac's syndrome, glaucoma, retinal detachment) in a subject, comprising any contrast or imaging agents including an indocyanine green (ICG) or fluorescein alone or in conjunction with a peptide or active agent. The agent is administered to a subject. The subject is a human or an animal and has a disease, injury, or malformation of ocular structures. Administration is intravenous, subcutaneous, intranasal, oral, intraperitoneal, intramuscular, intra-ocular, topical, or intradermal. Upon administration, the agent is targeted to vascular tissues and cells thereof or is selectively retained within the blood. The agent is then visualized using the imaging system, in conjunction with an ophthalmoscope, retinal or fundus camera system, optical coherence tomography (OCT) system, surgical microscope, or other ophthalmic imaging system. Ophthalmic angiogram of the choroid may similarly utilize the imaging system and/or methods disclosed herein. The imaging system enables the operator to view fluorescence and visible imaging together and simultaneously with the operating microscope or other imaging system. There is no need to reposition the operating microscope or other imaging system to view the fluorescence and visible images thus providing color imaging of the ocular structures together with the fluorescence imaging, which decreases disruption to the surgical or diagnostic workflow. Other contrast or imaging agents can be used as described herein.

### Example 5

### Use of System for Perfusion Imaging in Surgery

This example describes use of the imaging system and/or method disclosed herein for coaxial illumination and visualization of tissue perfusion in a subject. The imaging system of the present invention is used to image blood flow in tissues during surgeries requiring adequate perfusion to promote healing of joined tissues (e.g., anastomosis, reconstructive surgery, or plastic surgery). A contrast or imaging agent, including an indocyanine green (ICG) or fluorescein alone or in conjunction with a peptide or active agent, is administered to a subject. The subject is a human or an animal and has a condition such as occlusion, cancer, or trauma. Administration is intravenous, subcutaneous, intranasal, oral, intraperitoneal, intramuscular, or intradermal. Upon administration, the agent is targeted to vascular tissues and cells thereof, or is selectively retained within the blood or lymph. The agent is then visualized using the imaging system, in conjunction with a neurosurgical operating microscope, a neuroendoscope, a vascular endoscope, an endoscope, thoracoscope, telescope, robotic surgical system, other surgical microscope, or as an open imaging system. The selection of the appropriate imaging system is made by the surgeon and is dependent on the surgical approach. The surgeon exposes the occlusion, cancer, or other tissue, and the imaging system is initialized and used continuously. The imaging system enables the surgeon to view fluorescence and visible imaging together and simultaneously with the operating microscope or other imaging system. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 6

### Use of System for Detection of Plaque Instability and Restenosis in Atherosclerosis

This example describes use of the imaging system and/or method disclosed herein for coaxial illumination and visualization of atherosclerotic plaques and restenosis in a subject. The imaging system of the present invention is used to image atherosclerotic plaques within blood vessels in order to assess their stability, and to image blood flow through stented blood vessels for diagnosis of restenosis. A contrast or imaging agent, including an indocyanine green (ICG) or fluorescein alone or in conjunction with a peptide or active agent, is administered to a subject. The subject is a human or an animal and has atherosclerosis. Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with an endovascular endoscope, a vascular endoscope, an endoscope, thoracoscope, telescope, robotic surgical system, other surgical microscope, or as an open imaging system. The selection of the appropriate imaging system is made by the surgeon and is dependent on the surgical or diagnostic approach. The surgeon exposes the plaque, stent, or other tissue, and the imaging system is initialized and used continuously. The imaging system enables the surgeon to view fluorescence and visible imaging together and simultaneously with the operating microscope or other imaging system. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 7

### Use of System for Imaging Vital Organs or Structures

This example describes use of the imaging system and/or method disclosed herein for imaging vital organs or structures in a subject during surgery. The imaging system of the present invention is used to image contrast between vital organs or structures (e.g., kidney, ureters, thyroid, liver or liver segments, nerves) and other surrounding tissues. A contrast or imaging agent, including an indocyanine green (ICG), methylene blue, or fluorescein, alone or in conjunction with a peptide or active agent, is administered to a subject. The subject is a human or an animal and has a disease or condition that requires surgical intervention near vital organs or structures. Administration is intravenous, subcutaneous, intranasal, oral, intraperitoneal, intramuscular, intradermal, or by intratumor injection. Upon administration, the agent is targeted to vascular tissues and cells thereof, to vital organ tissues and cells thereof (e.g., nerves), or is selectively retained within the blood. The agent is then visualized using the imaging system, in conjunction with a laparoscope, a vascular endoscope, an endoscope, thoracoscope, telescope, robotic surgical system, other surgical microscope, or as an open imaging system. The selection of the appropriate imaging system is made by the surgeon and is dependent on the surgical or diagnostic approach. The surgeon exposes the area of interest, and the imaging system is initialized and used continuously. Contrast results either from differential blood flow to the organ or tissue (e.g., kidney contrasting with adrenal gland, thyroid contrasting with parathyroid, or liver segment following selective injection to an artery supplying the segment), from elimination pathways (e.g. ureters or kidney following administration of a dye or conjugate with renal clearance), or from selective targeting to the organ or structure (e.g., using a peptide that targets proteins found on nerve sheaths). The imaging system enables the surgeon to view fluorescence and visible imaging together and simultaneously with the operating microscope or other imaging system. The contrast enables the surgeon to avoid injury to normal tissues and to selectively remove organs, organ segments, or other tissues as appropriate. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 8

### Use of System for Diagnosis of Ischemia

This example describes use of the imaging system and/or method disclosed herein for imaging and diagnosis of tissue ischemia. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence of fluorescence signal from the tissue of interest indicates reduced or absent blood flow, and ischemia. Other contrast or imaging agents can be used as described herein.

### Example 9

### Use of System During Venography

This example describes use of the imaging system and/or method disclosed herein for imaging of veins and diagnosis of deep vein thrombosis (DVT) or other vein abnormalities. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and DVT or other vein abnormalities. Other contrast or imaging agents can be used as described herein.

### Example 10

### Use of System to Image and Monitor Cerebral Vascular Flow

This example describes use of the imaging system and/or method disclosed herein for imaging and diagnosis of vessel narrowing (stenosis), clot formation (thrombosis), blockage (embolism) or blood vessel rupture (hemorrhage) in the brain. Lack of sufficient blood flow (ischemia) affects brain tissue and may cause a stroke. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and ischemia. Other contrast or imaging agents can be used as described herein.

### Example 11

### Use of System to Image and Monitor Vascular Flow to Tumors

This example describes use of the imaging system and/or method disclosed herein for imaging of tumor vasculature for monitoring, diagnosis and treatment of tumors. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Presence of enhanced and abnormal fluorescence signal from the tissue of interest indicates angiogenesis, or stimulation of blood vessel growth indicative of tumors. Other contrast or imaging agents can be used as described herein.

### Example 12

### Use of System for Coronary Angiography, Angiogram and Cardiac Catheterization

During coronary angiography, a contrast or imaging dye is injected into a subject artery through a catheter or other. Using the system and methods herein blood flow is monitored through the subject's heart. This test is also known as a cardiac angiogram, catheter arteriography, or cardiac catheterization. This example describes use of the imaging system and/or method disclosed herein for imaging of heart vasculature. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has known or suspected coronary artery disease. Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and ischemia. Catheterization, angioplasty, plaque ablation, stent insertion or replacement, or other treatment can accompany the imaging. Other contrast or imaging agents can be used as described herein.

Similarly, during a coronary angiogram, a contrast or imaging dye is injected into the blood vessels of the heart. Using the system and methods herein blood flow is monitored through the subject's heart. The system is used to take a series of images (angiograms), to visualize the cardiovasculature and blood vessels feeding blood to the heart. Concurrent with imaging and monitoring of vessels using this method, clogged heart arteries can be opened (angioplasty) during the coronary angiogram. Coronary computed tomography angiography (CCTA) can also similarly employed.

### Example 13

### Use of System for Imaging and Monitoring Stroke, Coronary Artery Disease or Congestive Heart Failure

This example describes use of the imaging system and/or method disclosed herein for imaging and diagnosis of stroke, coronary artery disease or congestive heart failure, or in cardiography. Lack of sufficient blood flow (ischemia) affects brain tissue and may cause a stroke. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and ischemia indicative of stroke, coronary artery disease or congestive heart failure. Other contrast or imaging agents can be used as described herein.

### Example 14

### Use of System During Aneurysm, Arteriovenous Malformation, or Cavernous Malformation Resection

This example describes use of the imaging system and/or methods disclosed herein for coaxial illumination and visualization of tozuleristide fluorescence during surgical resection of an arteriovenous malformation or cavernous malformation in a pediatric patient. The imaging system of the present invention was used to image brain tissue to detect abnormal vasculature using fluorescence imaging. Surgery was performed to remove the abnormality from the subject.

A pediatric subject with a history of anosmia was found on MRI to have a 3.5 cm T1-hypointense, T2/FLAIR-hyperintense mass in the right middle frontal gyrus with a central enhancing nodule, initially diagnosed pre-operatively to be a low-grade glioma. The subject did not have any prior history of neurosurgery. The patient received 22 mg (15 mg/M²) of tozuleristide via IV injection approximately 5-6 hours before surgery and image collection. The imaging system head was attached to the Zeiss Pentero surgical microscope along with two eyepieces prior the start of surgery.

After the lesion was exposed, the imaging system was initialized and used continuously. The imaging system enabled the surgeon to view fluorescence and visible imaging together and simultaneously with the operating microscope. A microsurgical resection was performed through a right frontal craniotomy. The abnormal tissue had a dark blue mulberry appearance that fluoresced avidly with toluzeristide. The surrounding tissue showed no fluorescence. The abnormal tissue was completely resected. The patient recovered without deficit. Pathology confirmed the absence of tumor. Pathology data suggested the abnormality was vascular in nature and demonstrated that tozuleristide fluorescence was detected in a cerebral non-tumoral lesion. While ICG alone transiently lights up blood vessels immediately after injection, toluzeristide fluorescence is dependent on pathological tissue binding and uptake and can highlight diseased tissue up to 30 hours after injection.

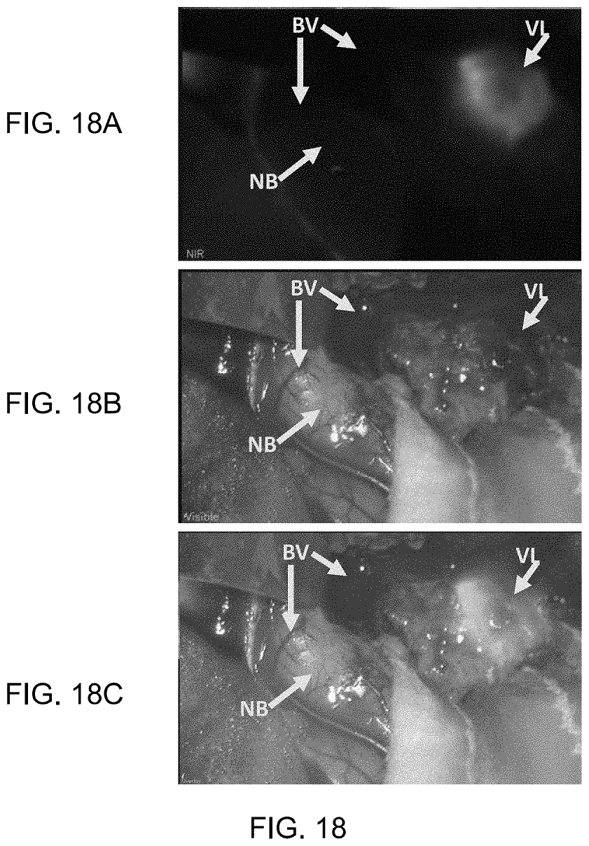

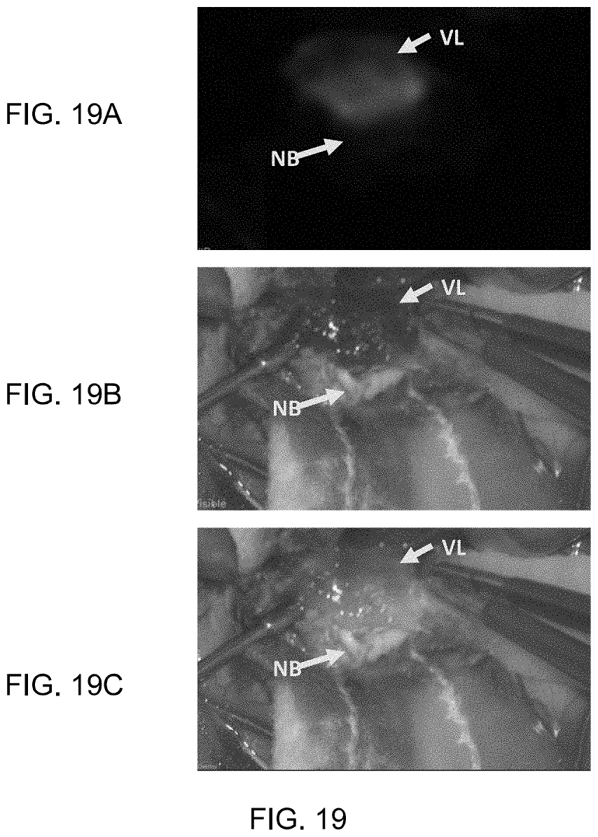

Video was captured for the duration of the resection, and still images were captured of the exposed lesion. Tozuleristide fluorescence was observed in situ in the exposed vascular lesion. FIGS. 18 and 19 show images taken from the vascular lesion with the near-infrared (NIR) fluorescence images of the vascular lesion using the imaging system. The vascular abnormality appeared to the surgeon as a bright blue-green mass (arrows labeled “VL”) in the NIR fluorescence image and in the overlay image (shown as a bright white mass in grey-scale), while the normal brain tissue (labeled “NB”) and vasculature (labeled “BV”) appeared darker than the vascular lesion in the NIR fluorescence image indicating no discernable background fluorescence in non-lesion or normal brain tissue or in normal vasculature. In the overlay image, the normal brain tissue and normal blood vessels appeared pink or light tan to red, as it does under normal visible light or white light. The surgeon noted that only the abnormal vascular tissue appeared fluorescent. The fluorescent tissue samples were demonstrated and confirmed to be non-cancerous and vascular in nature by histopathology.

FIG. 18 and FIG. 19 show representative images of in situ or intra-operative tissue during surgery on a vascular lesion in a patient, wherein 22 mg (15 mg/m²) of tozuleristide was administered to the human subject. FIG. 18A shows a near-infrared (NIR) image of the in situ specimen. Fluorescence signal, corresponding to lighter and brighter areas in the NIR images, is indicative of the presence of tozuleristide in the vascular lesion. Labeled arrows indicate non-fluorescent regions of normal blood vessels (“BV”) and normal brain tissue (“NB”). In contrast, fluorescence signal corresponding to lighter and brighter areas in the NIR image, was indicative of the presence of tozuleristide on the abnormal vascular lesion (“VL”), and not in normal tissue. FIG. 18B shows the white light image corresponding to FIG. 18A that represents what the surgeon would normally see without fluorescence guidance. The arrows mark the same locations as shown in the NIR image in FIG. 18A. The vascular lesion (“VL”) had a similar appearance to the normal blood vessels (“BV”) in this image. FIG. 18C shows the NIR fluorescence and white light composite image of FIG. 18A and FIG. 18B, with arrows marking the same locations as shown in FIG. 18A and FIG. 18B. Fluorescence in the vascular lesion (“VL”) clearly differentiated it from the surrounding normal tissues, including normal blood vessels (“BV”). FIG. 19A shows a near-infrared (NIR) image of the vascular lesion during the surgery. Arrows indicate the vascular lesion (labeled “VL”) and adjacent normal brain (labeled “NB”), which is non-fluorescent. FIG. 19B shows the white light image corresponding to FIG. 19A. While the normal brain has a light tan to pink color (light gray in a gray scale image), it is perfused with normal blood vessels that can be differentiated from the vascular lesion by the absence of fluorescence. FIG. 19C shows the composite white light and NIR image shown in FIG. 19A and FIG. 19B.

The fluorescent tissue samples were demonstrated and confirmed to be non-cancerous and vascular in nature by histopathology. The pathology did not indicate cancer or neoplastic abnormalities but rather confirmed a vascular abnormality that did not indicate cancer. Intraoperative pathology was performed on two specimens. One showed dilated vessels most compatible with vascular malformation, and the other was normal brain parenchyma with no evidence of neoplasm. Post-operative pathology was performed on 19 excised specimens with the numbered annotations for reference are shown below in TABLE 3.

TABLE 3
Pathology Specimens

| Specimen | | |
|---|---|---|
| Number | Description | Observation |
| 1 | Right brain specimen 1 (Excision) | Blood vessels with hyalinization and |
| | | chronic inflammation |
| 2 | Right brain specimen 2 (Excision) | Gray and white matter and blood vessels |
| | | with chronic inflammation, and |
| | | microcalcifications |
| 3 | Right brain specimen 3 (Excision) | Blood vessels, interspersed neuropil, |
| | | chronic inflammation, and calcifications |
| 4 | Right brain specimen 4 (Excision) | Blood vessels with chronic inflammation |
| | | and interspersed gray matter |
| 5 | Right brain specimen 5 (Excision) | Blood vessels with interspersed gray and |
| | | white matter and proteinaceous aggregates |
| 6 | Right brain specimen 6 (Exision) | Minute cluster of blood vessels |
| 7 | Deep lateral equivocal tissue | Minute fragment of white matter |
| | specimen 7 (Excision) | |
| 8 | Posterior equivocal tissue | Gray matter with focus of blood vessels |
| | specimen 8 (Excision) | |
| 9 | Inferior equivocal tissue | Minute fragment of gray matter |
| | specimen 9 (Excision) | |
| 10 | Anterior equivocal tissue | Minute fragment of gray and white matter |
| | specimen 10 (Excision) | |
| 11 | Anterior lateral equivocal tissue | Minute fragments of gray and white matter |
| | specimen 11 (Excision) | with focus of blood vessels |
| 12 | Anterior equivocal tissue | Minute fragment of gray matter |
| | specimen 12 (Excision) | |
| 13 | Posterior equivocal tissue | Minute fragments of gray and white matter |
| | specimen 13 (Excision) | |
| 14 | Deep lateral equivocal tissue | Minute fragment of white matter |
| | specimen 14 (Excision) | |
| 15 | Anterior equivocal tissue | Minute fragments of gray and white matter |
| | specimen 15 (Excision) | |
| 16 | Inferior equivocal tissue | Minute fragments of gray matter with |
| | specimen 16 (Excision) | reactive changes |
| 17 | Anterior lateral equivocal tissue | Small fragments of gray and white matter |
| | specimen 17 (Excision) | |
| 18 | Right deep specimen 18 | Small fragments of gray matter with |
| | (Excision) | reactive surgical changes |
| 19 | Right deep specimen 19 | Small fragment of gray and white matter |
| | (Excision) | |

Specimens with substantial vascular components were considered for examination. In specimen 8 the vessels were not separated by neuropil. In specimens 3, 4, and 5, neuropil intervened between the vessels, indicating an overall diagnosis of vascular malformation.

This case demonstrated that the imaging system could be used continuously in an intraoperative setting to capture images and video of white light and NIR fluorescence, without disrupting the normal surgical flow. The data further demonstrated that the coaxial illumination and imaging system enabled the surgeon to visualize and precisely localize fluorescence in non-neoplastic pathologies during surgery and use this information to remove abnormal vascular tissue during resection. The systems and methods herein can be used to detect, image and treat cavernous malformation, cavernous angiomas, cavernous hemangiomas, or cerebral cavernous malformation (CCM), and arteriovenous malformation, including via surgery.

### Example 15

### Use of System for Imaging and Monitoring Occlusion of Veins or Arteries Resulting in Organ Failure or Injury

This example describes use of the imaging system and/or methods disclosed herein for imaging and diagnosis of occlusion of arteries or veins or detection of hemorrhage or embolism in a variety of organ systems, including brain, heart, lung, kidney, liver, pancreas, or in the extremities (e.g., legs, neck, and arms). Lack of sufficient blood flow (ischemia) affects tissue and may cause organ damage or organ failure, hemorrhagic stroke, and the like. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and ischemia indicative of organ damage or organ failure, hemorrhagic stroke, and the like. Other contrast or imaging agents can be used as described herein.

### Example 16

### Use of Compounds and Methods During Aneurysm, Arteriovenous Malformation, or Cavernous Malformation Resection Cavernous Malformation Resection

This example describes use of the compounds and methods disclosed herein for coaxial illumination and visualization of tozuleristide fluorescence during surgical resection of a cavernous malformation in a pediatric patient. An imaging system was used to image brain tissue to detect abnormal vasculature using fluorescence imaging. Surgery was performed to remove the abnormality from the subject.

A pediatric subject with anxiety and depression presented to the emergency room after smoking a cigar and marijuana at a party. She lost consciousness and awoke with a headache. On exam she was lethargic with a flat affect without focal neurological deficit. Medications included fluoxetine and hydroxyzine. MRI showed a 3 cm mass in the left cerebellar hemisphere with blooming artifact and minimal enhancement. The clinical impression was a cerebral cavernous malformation, likely incidental. The decision was made to have it removed.

The patient received 15 mg/m2 of a fluorescent conjugate comprising SEQ ID NO: 1 conjugated to an ICG (tozuleristide, also referred to herein as Compound 76) via IV injection on the morning of surgery and image collection. The imaging system head was attached to the Zeiss Pentero surgical microscope along with two eyepieces prior the start of surgery.

After the lesion was exposed, the imaging system was initialized and used continuously. The imaging system enabled the surgeon to view fluorescence and visible imaging together and simultaneously with the operating microscope. A microsurgical resection was performed through a left retroaricular craniotomy with frameless stereotactic navigation and intraoperative ultrasonography. A round mulberry appearing vascular lesion that contained areas of thrombosed vessels and firm calcifications was encountered. The lesion fluoresced avidly with tozuleristide. The surrounding cerebellum was discolored yellow, presumably from prior hemorrhage, and did not fluoresce. The lesion was removed. The patient awakened with mild left upper extremity dysmetria that had resolved at the time of discharge on postoperative day 5. Postoperative MRI confirmed resection of the mass. Pathology showed a cavernous malformation with abundant abnormal vessels, scattered inflammation and some thrombosed vessels.

This case demonstrated that the compounds and methods could be used in an intraoperative setting and enabled the surgeon to visualize and precisely localize fluorescence in non-neoplastic pathologies during surgery and use this information to remove abnormal vascular tissue during resection.

This case further demonstrated that the imaging system could be used continuously in an intraoperative setting to capture images and video of white light and NIR fluorescence, without disrupting the normal surgical flow. The data further demonstrated that the coaxial illumination and imaging system enabled the surgeon to visualize and precisely localize fluorescence in non-neoplastic pathologies during surgery and use this information to remove abnormal vascular tissue during resection. The systems and methods herein can be used to detect, image and treat cavernous malformation, cavernous angiomas, cavernous hemangiomas, or cerebral cavernous malformation (CCM), and arteriovenous malformation, including via surgery.

While certain embodiments of the present disclosure have been exemplified or shown and described herein, it will be apparent to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all embodiments of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed:

1. A method of imaging a vasculature or structure in a sample from a subject, the method comprising producing an image of the vasculature or structure by imaging fluorescence using an imaging system, the system comprising:
i) one or more detectors configured to form a fluorescence image of the sample and form a visible image of the sample;
ii) a light source configured to emit an excitation light to induce fluorescence from the sample; and
iii) a plurality of optics arranged to:
direct the excitation light toward the sample; and
direct a fluorescent light and a visible light from the sample to the detector;
wherein the excitation light and the fluorescence light are directed substantially coaxially.

2. The method of claim 1, wherein the fluorescence imaged is autofluorescence, a contrast or imaging agent, or any combination thereof.

3. The method of any one of claim 1 or 2, wherein the method further comprises administering a contrast or imaging agent to the subject.

4. A method of imaging a vasculature or structure in a sample from a subject, the method comprising:
a) administering a contrast or imaging agent to the subject;
b) producing an image of the vasculature or structure by imaging the contrast or imaging agent using an imaging system, the system comprising:
i) one or more detectors configured to form a fluorescence image of the sample and form a visible image of the sample;
ii) a light source configured to emit an excitation light to induce fluorescence from the sample; and
iii) a plurality of optics arranged to:
direct the excitation light toward the sample; and
direct a fluorescent light and a visible light from the sample to the detector;
wherein the excitation light and the fluorescence light are directed substantially coaxially.

5. The method of any one of claims 1-4, wherein the excitation light comprises infrared light.

6. The method of claim 5, wherein the infrared light comprises near infrared light.

7. The method of any one of claims 5 to 6, wherein the plurality of optics comprises a dichroic shortpass beam splitter to direct the infrared light and the visible light to the detector.

8. The method of any one of claims 1 to 7, wherein the detector comprises a plurality of detectors and wherein the visible image comprises a color image.

9. The method of claim 8, wherein the plurality of detectors comprises a first detector to generate a color image and a second detector to generate the infrared image.

10. The method of any one of claims 1 to 9, wherein the imaging system further comprises:
a) a laser;
b) an optical light guide coupled to a laser or narrow-band light source;
c) a collimating lens into which the optical light guide ends;
d) a laser clean-up filter configured to reduce bandwidth of the excitation light;
e) a dielectric mirror;
f) a diffuser;
g) a hole; or
h) any combination thereof.

11. The method of any one of claims 1 to 10, wherein the light source emits a wavelength absorbed by a fluorophore.

12. The method of any one of claims 1 to 11, wherein the light source is a narrow-band light source.

13. The method of claim 12, wherein the narrow-band light source generates light with a wavelength of from 700 nm to 800 nm, from 650 to 900 nm, from 700 nm to 900 nm, from 340 nm to 400 nm, from 360 to 420 nm, from 380 nm to 440 nm, or from 400 nm to 450 nm.

14. The method of any one of claim 12 or 13, wherein the narrow-band light source emits light with a wavelength visible by an NIR camera, and wherein the system further comprises a lens coupled to the optical light guide.

15. The method of any one of claims 10 to 14, wherein the laser generates light with a wavelength of from 650 nm to 4000 nm, from 700 nm to 3000 nm, or from 340 nm to 450 nm.

16. The method of any one of claims 10 to 15, wherein the laser generates light with a wavelength of from 750 nm to 950 nm, from 760 nm 825 nm, from 775 nm to 795 nm, from 780 nm to 795 nm, from 785 nm to 795 nm, from 780 nm to 790 nm, from 785 nm to 792 nm, or from 790 nm to 795.

17. The method of any one of claims 10 to 16, wherein the collimating lens is configured to collimate the excitation light, the fluorescent light, and the visible light.

18. The method of any one of claims 10 to 17, wherein the optical light guide is a fiber optic cable, a solid light guide, a plastic light guide, a liquid light guide, a waveguide, or any combination thereof.

19. The method of any one of claims 10 to 18, wherein the laser clean-up filter is configured to reduce bandwidth of the excitation light.

20. The method of any one of claims 1 to 19, wherein the light source comprises:
a) a broadband light source;
b) an optical light guide coupled to the broadband light source; or
c) a combination of a) and b).

21. The method of claim 20, wherein the broadband light source comprises one or more LEDs, a Xenon bulb, a halogen bulb, one or more or lasers, sunlight, fluorescent lighting, or a combination thereof.

22. The method of any one of claim 20 or 21, wherein the broadband light source emits a visible wavelength, a wavelength absorbed by a fluorophore, or a combination thereof.

23. The method of any one of claims 20 to 22, wherein the broadband light source emits light with a wavelength visible by an NIR camera and wherein the system further comprises a lens coupled to the optical light guide.

24. The method of any one of claims 1 to 23, wherein the imaging system comprises a plurality of light sources and further comprises one or more of the following to combine the plurality of light sources into a single coaxial path:
a) an optical attenuator comprising a dichroic filter, a dichroic mirror, a shutter, or any combination thereof;
b) a filter at each light source
c) a clean-up filter for a wavelength range of the excitation light;
d) a short-pass filter for a wavelength range of the excitation light;
e) an optical light guide; or
f) an illumination optic.

25. The method of any one of claims 1 to 24 further comprising:
a) a shortpass (SP) mirror;
b) a longpass (LP) mirror;
c) a combination thereof.

26. The method of any one of claims 10 to 25, wherein the dielectric mirror is configured to reflect the excitation light such that excitation light and the reflected excitation light have an intersection angle of from about 60 degrees to about 120 degrees.

27. The method of claim 26, wherein the dielectric mirror is configured to reflect the excitation light such that excitation light and reflected excitation light have an intersection angle of about 90 degrees.

28. The method of any one of claims 10 to 27, wherein the diffuser is configured to diffuse the excitation light.

29. The method of any one of claims 10 to 28, wherein the hole is configured to let pass at least part of the excitation light.

30. The method of any one of claims 10 to 29, wherein the hole is in a near-infrared mirror.

31. The method of any one of claims 10 to 30, wherein the hole has a shape, and a size, and wherein at least one of the shape of the hole and the size of the hole are configured to allow an even distribution illumination of the sample within a field of view of a microscope.

32. The method of any one of claims 1 to 31, wherein the excitation light comprises blue or ultraviolet light.

33. The method of claim 32, wherein the blue or ultraviolet light comprises a light having a wavelength of from about 10 nm to about 460 nm, from about 10 nm to about 400 nm, or from about 400 nm to about 460 nm.

34. The method of any one of claims 1 to 33, wherein the plurality of optics comprises a dichroic shortpass beam splitter, wherein the dichroic shortpass beam splitter is configured to let pass light with a wavelength of at most 700 nm with 90% to 95% efficiency at one or more specified angles of incidence.

35. The method of claim 34, wherein the one or more specific angles is within a range from 30 to 150 degrees.

36. The method of any one of claims 1 to 35, wherein the visible light is directed from a microscope, an endoscope, an exoscope, a surgical robot, or an operating room lighting external to the imaging system.

37. The method of claim 36, further comprising a locking key configured to securely lock an imaging head onto the microscope.

38. The method of any one of claims 1 to 37, wherein the plurality of optics further comprises a secondary dichroic shortpass beam splitter.

39. The method of any one of claims 1 to 38, wherein the system further comprises a dichroic longpass beam splitter.

40. The method of any one of claims 7 to 39, wherein the excitation light and the fluorescence light substantially overlap at the beam splitter.

41. The method of any one of claims 1 to 40, wherein substantially coaxial comprises an intersection angle of two optical paths to be less than 20 degrees, 15 degrees, 10 degrees, 5 degrees, 2 degrees, or 1 degree.

42. The method of any one of claims 1 to 41, further comprising a physical attenuator configured to block an ambient light from one, two, or more of: the detector, the light source, and the plurality of optics.

43. The method of claim 42, wherein the physical attenuator comprises a shield, a hood, a sleeve, a light shroud, or a baffle.

44. The method of any one of claims 1 to 43, wherein the imaging system further comprises an Application Specific Integrated Circuit (ASIC) or a processor, wherein at least one of the ASIC and the processor is configured with instructions to generate a composite image of the sample, the composite image comprising the fluorescence image overlaid with the visible image.

45. The method of any one of claims 1 to 44, wherein the contrast or imaging agent comprises a dye, a fluorophore, a fluorescent biotin compound, a luminescent compound, a chemiluminescent compound, or any combination thereof.

46. The method of any one of claims 1 to 45, wherein the contrast or imaging agent absorbs a wavelength between from about 200 mm to about 900 mm.

47. The method of any one of claims 1 to 46, wherein the contrast or imaging agent comprises DyLight-680, DyLight-750, VivoTag-750, DyLight-800, IRDye-800, VivoTag-680, Cy5.5, or an indocyanine green (ICG) and any derivative of the foregoing; fluorescein and fluorescein dyes (e.g., fluorescein isothiocyanineo rF ITC, naphthofluorescein, 4′, 5′-dichloro-2′,7′-dimethoxyfluorescein, 6-carboxyfluorescein or F AM, etc.), carbocyanine, merocyanine, styryl dyes, oxonol dyes, phycoerythrin, -111-rythrosine, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin, coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), 7-aminocoumarin, a dialkylaminocoumarin reactive dye, 6,8-difluoro-7-hydroxycoumarin fluorophore, a hydroxycoumarin derivative, an alkoxycoumarin derivatives, a succinimidyl ester, a pyrene succinimidyl ester, a pyridyloxazole derivative, an aminonaphthalene-based dyes, dansyl chlorides, a dapoxyl dye, Dapoxyl sulfonyl chloride, amine-reactive Dapoxyl succinimidyl ester, carboxylic acid-reactive Dapoxyl (2-aminoethyl)sulfonamide), a bimane dye, bimane mercaptoacetic acid, an NBD dye, a QsY 35, or any combination thereof.

48. The method of any one of claims 1 to 47, wherein the administering comprises intravenous administration, intramuscular administration, subcutaneous administration, intraocular administration, intra-arterial administration, peritoneal administration, intratumoral administration, intradermal administration, or any combination thereof.

49. The method of any one of claims 1 to 48, wherein the imaging comprises tissue imaging, ex vivo imaging, intraoperative imaging, or any combination thereof.

50. The method of any one of claims 1 to 49, wherein the sample is in an in vivo sample, an ex vivo sample, or an intraoperative sample.

51. The method of any one of claims 1 to 50, wherein the sample is an organ, an organ substructure, a tissue, or a cell.

52. The method of any one of claims 1 to 51, wherein the sample emits autofluorescence.

53. The method of claim 52, wherein the autofluorescence of the sample is produced by an ocular fluorophore, tryptophan, or protein present in a tumor or malignancy.

54. The method of any one of claims 1 to 53, wherein the method is used to visualize vessel flow or vessel patency.

55. The method of any one of claims 1 to 54, wherein the vasculature or structure comprises a blood vessel, lymph vasculature, neuronal vasculature, or CNS structure.

56. The method of any one of claims 1 to 55, wherein the imaging is angiography, arteriography, lymphography, or cholangiography.

57. The method of any one of claims 1 to 56, wherein the imaging comprises detecting a vascular abnormality, vascular malformation, vascular lesion, organ or organ substructure, cancer or diseased region, tissue, structure or cell.

58. The method of claim 57, wherein the vascular abnormality, vascular malformation, or vascular lesion is an aneurysm, an arteriovenous malformation, a cavernous malformation, a venous malformation, a lymphatic malformation, a capillary telangiectasia, a mixed vascular malformation, a spinal dural arteriovenous fistula, or a combination thereof.

59. The method of any one of claim 57 or 58, wherein the organ or organ substructure is brain, heart, lung, kidney, liver, or pancreas.

60. The method of any one of claims 1 to 59, further comprising performing surgery on the subject.

61. The method of claim 60, wherein the surgery comprises angioplasty, cardiovascular surgery, aneurysm repair, valve replacement, aneurysm surgery, arteriovenous malformation or cavernous malformation surgery, venous malformation surgery, lymphatic malformation surgery, capillary telangiectasia surgery, mixed vascular malformation surgery, or a spinal dural arteriovenous fistula surgery, repair or bypass, arterial bypass, organ transplant, plastic surgery, eye surgery, reproductive system surgery, stent insertion or replacement, plaque ablation, removing the cancer or the diseased region, tissue, structure or cell of the subject, or any combination thereof.

62. The method of any one of claims 1 to 61, wherein the imaging comprises imaging a vascular abnormality, cancer or diseased region, tissue, structure, or cell of the subject after surgery.

63. The method of any one of claims 1 to 62, further comprising treating a cancer in the subject.

64. The method of any one of claims 1 to 63, further comprising repair of an intracranial CNS vascular defect, a spinal CNS vascular defect; peripheral vascular defects; removal of abnormally vascularized tissue; ocular imaging and repair; anastomosis; reconstructive or plastic surgery; plaque ablation or treatment or restenosis in atherosclerosis; repair or resection (including selective resection), preservation (including selective preservation), of vital organs or structures such as nerves, kidney, thyroid, parathyroid, liver segments, or ureters; identification and management (sometimes preservation, sometimes selective resection) during surgery; diagnosis and treatment of ischemia in extremities; or treatment of chronic wounds.

65. The method of claim 64, wherein the intracranial vascular defect and/or the spinal vascular defect comprises an aneurysm, an arteriovenous malformation, a cavernous malformation, a venous malformation, a lymphatic malformation, a capillary telangiectasia, a mixed vascular malformation, or a spinal dural arteriovenous fistula, or any combination thereof.

66. The method of claim 64, wherein the peripheral vascular defect comprises an aneurysm, a coronary bypass, an other vascular bypass, a cavernous malformation, an arteriovenous malformation, a venous malformation, a lymphatic malformation, a capillary telangiectasia, a mixed vascular malformation, a spinal dural arteriovenous fistula, or any combination thereof.

67. The method of claim 64, wherein the abnormally vascularized tissue comprises endometriosis or a tumor.

68. The method of any one of claims 1 to 67, wherein the method further comprises radiology or fluorescence imaging using one or more of: an X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), microscope, operating microscope, confocal microscope, fluorescence scope, exoscope, surgical robot, surgical instrument, or any combination thereof.

69. The method of any one of claims 1 to 68, wherein the method comprises measuring and/or quantitating fluorescence using one or more of a microscope, a confocal microscope, a fluorescence scope, an exoscope, a surgical robot, a surgical instrument, or any combination thereof.

70. The method of any one of claims 1 to 69, wherein the imaging system is combined with or integrated into: a microscope, a confocal microscope, a fluorescence scope, an exoscope, a surgical robot, a surgical instrument, or any combination thereof.

71. The method of any one of claims 1 to 69, wherein the imaging system comprises a microscope, a confocal microscope, a fluorescence scope, an exoscope, a surgical robot, a surgical instrument, or any combination thereof.

72. The method of any one of claims 69 to 71, wherein at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800 (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., RetinaVue 100, RetinaVue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPAL1 NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

73. The method of any one of claims 1 to 72, wherein the imaging system is configured to detect, image or assess a therapeutic agent; detect, image or assess a safety or a physiologic effect of the companion diagnostic agent; detect, image or assess a safety or a physiologic effect of the therapeutic agent; detect, image or assess a safety or a physiologic effect of the companion imaging agent; or any combination thereof.

74. The method of any one of claims 1 to 73, wherein the contrast or imaging agent's safety or physiologic effect is bioavailability, uptake, concentration, presence, distribution and clearance, metabolism, pharmacokinetics, localization, blood concentration, tissue concentration, ratio, measurement of concentrations in blood or tissues, therapeutic window, visibility window, range and optimization, or any combination thereof.

75. The method of any one of claims 1 to 74, wherein the method comprises administering a companion diagnostic agent, a therapeutic agent, or an imaging agent, and wherein the imaging comprises detecting the companion diagnostic agent, the therapeutic agent, or the imaging agent.

76. The method of claim 75, wherein the companion diagnostic agent, the therapeutic agent, or the imaging agent comprises a chemical agent, a radiolabel agent, radiosensitizing agent, fluorophore, therapeutic agent, an imaging agent, a diagnostic agent, a protein, a peptide, or a small molecule.
